(19) United States
(12) Patent Application Publication
(10) Pub. No.: US 2022/0185785 A1
(43) Pub. Date: Jun. 16, 2022

(54) INHIBITORS OF CD40-CD154 BINDING

(71) Applicant: TONIX PHARMACEUTICALS HOLDING CORP, New York, NY (US)

(72) Inventors: Darryl RIDEOUT, Milford, PA (US); Seth LEDERMAN, New York, NY (US)

(73) Assignee: TONIX PHARMACEUTICALS HOLDING CORP, New York, NY (US)

(21) Appl. No.: 17/603,260

(22) PCT Filed: Apr. 13, 2020
(86) PCT No.: PCT/US2020/028002

§ 371 Date: Oct. 12, 2021

Related U.S. Application Data
(60) Provisional application No. 62/833,473, filed on Apr. 12, 2019.

(51) Int. Cl. C07D 257/04; C07D 403/12; C07D 403/10; C07D 285/135; C07D 275/06; C07D 213/81; C07D 249/06; C07D 307/68; C07C 237/42; C07C 311/51; C07C 317/44; C07D 333/38; C07D 231/56; C07D 211/34; C07D 213/52; C07C 233/80; C07C 259/10; C07D 213/75; C07D 237/20; C07D 401/14; C07D 487/04

(52) U.S. Cl. CPC C07D 257/04; C07D 487/04; C07D 403/10; C07D 285/135; C07D 275/06; C07D 213/81; C07D 249/06; C07D 307/68; C07C 237/42; C07C 311/51; C07C 317/44; C07D 333/38; C07D 231/56; C07D 211/34; C07D 213/52; C07C 233/80; C07C 259/10; C07D 213/75; C07D 237/20; C07D 401/14; C07D 403/12

(57) ABSTRACT

Disclosed herein are compounds including pharmaceutically acceptable salts, esters, prodrugs, hydrates and tautomers thereof which modulate the interactions of CD-40-CD40L. The compounds are useful for treating, ameliorating or preventing an autoimmune disease, inflammatory disease, or other immune-related disease in a patient in need of treatment.

### RELATED APPLICATIONS

This application claims the benefit of priority to U.S. Ser. No. 62/833,473, filed on Apr. 12, 2019.

### FIELD OF THE DISCLOSURE

The present disclosure is in the field of the treatment of autoimmune, other immune system related diseases and inflammatory diseases with small molecules that inhibit CD40-CD154 binding, pharmaceutical compositions containing the same, and methods of treating diseases using the same.

### BACKGROUND OF THE DISCLOSURE

CD154 (aka as CD40L, TNFSF5) is expressed on activated T lymphocytes and, through interactions with its receptor CD40 (TNFRSF5), plays a pivotal role in regulating the interplay between T cells and other cell types. CD154 contributes to the potentiation of autoimmune diseases and holds promise as a therapeutic and preventative target in autoimmune diseases such as systemic lupus erythematosus (SLE), rheumatoid arthritis, ankylosing spondylitis, lupus nephritis, Goodpasture's disease, Sjögren's syndrome, polymyositis, dermatomyositis, psoriasis, temporal arteritis, Churg-Strauss syndrome, multiple sclerosis, Guillain-Barré syndrome, transverse myelitis, myasthenia gravis, Addison's disease, thyroiditis, coeliac disease, ulcerative colitis, Crohn's disease, sarcoidosis, hemolytic anemia, idiopathic thrombocytopenic purpura, Behçet's disease, primary biliary cirrhosis, autoimmune diabetes, type I diabetes, Juvenile diabetes and blockade of CD154 has been shown to be highly efficacious in several inflammatory and autoimmune model systems. CD154 has also been suggested to play a role in the inflammatory aspects of atherosclerosis and neurodegenerative disorders and holds promise as a therapeutic and preventative target in atherosclerotic conditions such as angina pectoris, myocardial infarction and in neurodegenerative conditions, such as Alzheimer's disease, traumatic brain injury (TBI), chronic traumatic encephalitis (CTE), Parkinson's disease. In addition, CD154 is suggested to play a role in the rejection of transplanted solid organs and holds promise as a target in the prevention and treatment of acute and chronic rejection in bone marrow transplantation (and graft versus host disease) and of acute and chronic rejection in orthotopic and heterotopic solid organ transplants (e.g., kidney, heart, liver, lung, cornea, pancreas, pancreatic islets, pancreatic islet-cells), including xenotransplantation and transplants facilitated by pre-treatment/engraftment with donor bone marrow. CD154 also may play a role in the malignant transformation of cells and holds promise as a target in the prevention and treatment of hematologic and solid organ malignancies. The compounds described herein in some cases work better in treating cancer than protein inhibitors of CD154 because the tumor microenvironment is sometimes compartmentalized and inaccessible to protein therapeutics, and also because protein therapeutics may have pH dependent binding and may not function in tumor microenvironment where the pH can be low.

Anti-CD154 mABs have been associated with thrombosis which may have been caused by the interaction of CD154 on platelets and/or formation of immune complexes from soluble CD154, and the interaction anti-CD154 coated platelets or anti-CD154:solute CD154 immune complexes with Fc receptors on effector cells and possibly endothelial cells (Pinelli and Ford, Immunotherapy (2015); 7(4):399-410). In some cases, these potential problems could be avoided with small molecules which don't interact with Fc receptors. In addition, small molecules can enter the brain through the blood-brain barrier, whereas protein therapeutics generally cannot. Still further small molecules can potentially be given by mouth, or provided in depot injections. Additionally, small molecules can have better stability for longer storage life. Small molecules are less expensive to synthesize and purify reproducibly, less likely to elicit allergic responses, and more amenable to optimization of ADMET through minor alterations in structure and the use of prodrugs. There are more options for effective formulation of small molecules (e.g. to improve solubility in water, salt forms) as compared to proteins.

### DETAILED DESCRIPTION OF THE EMBODIMENTS

In one aspect, disclosed herein are compounds of Formula I

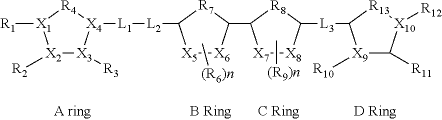

and pharmaceutically acceptable salts, esters, prodrugs, hydrates and tautomers thereof, wherein:

Ring A of Formula I is an optionally substituted 6-membered or 5-membered aryl, cycloalkyl, heteroaryl cycloalkyl, cycloalkenyl, or heterocycloalkyl ring.

In some embodiments Ring A is phenyl, 1,3,4-thiadiazole, 1,2,3-triazole, 1,2,4-triazole, piperidine, all of which can be optionally substituted.

In some embodiments, Ring A is benzo[d]isothiazol-3(2H)-one 1,1-dioxide or 2,3-dihydro-1H-isoindole-1,3-dione.

In some embodiments Ring A is 1,2,3-triazole.

In some embodiments Ring A is 1,2,4-triazole.

X₁, X₂, X₃, X₄ of Ring A are each separately and independently selected from the group consisting of C, or N;

In some embodiments, X₁, X₂, X₃, X₄ are all C. In some embodiments, X₁, X₂, X₃ are carbon and X₄ is N;

R₄ of Ring A is selected from the group consisting of CH═CH, CH, S, O, N, N═CH, CH═N, N═N, and CH₂CH₂;

In some embodiments, R₄ can be optionally substituted with a 5-membered heteroaryl ring. In some embodiments, the heteroaryl ring is a triazole or tetrazole. In some embodiments, the triazole is 1,2,3-triazole. In some embodiments the tetrazole is 1,2,4-triazole

R₁ of Ring A is selected from the group consisting of SO₂NR′₂, SO₂R′, COR′, COOR′, CON(R′)₂, CON(OR′)R′, tetrazole, triazole, C₁-C₃ alkyl chain, a 6-membered or 5-membered aryl, a 5 or 6-membered cycloalkyl, a 5- or 6-membered heterocycloalkyl, or 6-membered or membered heteroaryl optionally linked to the A ring through a bond; wherein each R′ is independently C₁-C₆ alkyl, C₂-C₆ heteroalkyl, 2-methoxyethyl, 2′-(2-methoxyethoxy)ethyl wherein each R′ can be optionally substituted with one or more groups selected from fluorine, C₁-C₄heteroalkyl, and ═O;

In some embodiments, R₁ is H when R₄ of Ring A is optionally substituted.

In some embodiments of R₁, the 6-membered or 5-membered aryl is phenyl, and the 6-membered or 5-membered heteroaryl is triazole, tetrazole, or furan which all can be optionally substituted.

In some embodiments of R₁, when R₁ is triazole, tetrazole, or furan, the triazole, tetrazole, or furan can be optionally substituted with C₁-C₆ alkyl, C₁-C₆ alkenyl, C₁-C₆ alkynyl or COR′, wherein the alkyl, alkenyl or alkynyl can be substituted with a C₃-C₆ cycloalkyl.

In some embodiments of R₁, when R₁ is phenyl, the phenyl can be optionally substituted with SO₂NR′₂, COR′, COOR′, CON(R′)₂, CON(OR′)R′, SO₂R′, tetrazole, or triazole, wherein R′ is as described above.

In some embodiments of R₁, the phenyl is independently substituted with COOCH₃, CON(CH₃)₂, 5-ethyl-2H-tetrazol or (3-(2-methoxyethoxy)prop-1-yn-1-yl.

In some embodiments of R₁, R₁ is piperidine that is optional substituted with SO₂NR′₂, COR′, COOR′, CON(R′)₂, CON(OR′)R′, SO₂R′, tetrazole, or triazole where R′ is defined as above. In some embodiments, the piperidine is substituted with COOCH₃.

In some embodiments of R₁, R₁ is furan optionally substituted with SO₂NR′₂, COR′, COOR′, CON(R′)₂, CON(OR′)R′, SO₂R′ where is R′ is defined as above, In some embodiments, the furan is substituted with COOCH₃

In some embodiments, R₂ of Ring A is H, optionally substituted C₁-C₃ alkylSO₂R′, SO₂NR′₂, COOR′, CON(R′)₂, CON(OR)R′, C₁-C₆ alkenyl, C₁-C₆ alkynyl, tetrazole, or triazole linked to the A ring through a bond, wherein each R′ is independently H, C₁-C₆ alkyl, C₃-C₉ cycloalkyl-alkyl, or C₂-C₁₃ heteroalkyl (in which 1 to 4 carbons are replaced with oxygen), wherein R′ can be optionally substituted with one or more groups selected from fluorine, or CH₃;

In some embodiments of R₂, the tetrazole or triazole is optionally substituted with C₁-C₆ alkyl or C₄-C₁₀ oxa-alkyl, dioxa-alkyl, or trioxa-alkyl. In some embodiments, the C₁-C₆ alkyl is optionally substituted with a 3-6cyclo-alkyl at its terminal carbon.

In some embodiments of R₂, the tetrazole can be optionally substituted with C₁-C₆ alkyl, C₃-C₉ cycloalkyl-alkyl, or C₂-C₁₃ oxa-alkyl (in which 1 to 4 carbons are replaced with oxygen).

In some embodiments, R₃ is selected from H, F, CH₃, 2-alkyl-ethynyl (C₁-C₄ alkyl) or (3-(2-methoxyethoxy)prop-1-yn-1-yl. In some embodiments, the C₁-C₄ alkyl is optionally substituted with C₃-C₆ cycloalkyl at the C-terminus. In some embodiments, the alkyl and cycloalkyl are optionally further substituted on carbon with one or more fluorine atoms.

In some embodiments, R₁ and R₂ taken together, form a fused ring with Ring A to form benztriazole, always substituted on either the 1- or 2-nitrogen with C₁-C₃ alkyl, 2-methoxyethyl, 2-(2′-methoxyethoxy-ethyl), (CH₂)_{w}COOR′, (CH₂)_{w}CON(OR′)R′, wherein R′ is C₁-C₃ alkyl and w is 0-3.

In some embodiments, R₁ and R₂ taken together, form a fused ring with Ring A to form benzo[d]isothiazol-3(2H)-one 1,1-dioxide or 2,3-dihydro-1H-isoindole-1,3-dione optionally substituted on nitrogen with C₁-C₃ alkyl, 2-methoxyethyl, 2-(2′-methoxyethoxy-ethyl), (CH₂)_{w}COOR′, (CH₂)_{w}CON(OR′)R′, wherein R′ is C₁-C₃ alkyl and w is 0-3.

In some embodiments, L₁ is absent, a single bond, —NHCO—, —CONH—, 1,3,4-thiadiazole-2,5-diyl or forms a ring with R₃;

In some embodiments, R₃ and L₁ taken together, along with the two intervening atoms to which they are attached, form an optionally substituted heterocycloalkyl ring having 1-3 heteroatoms independently selected from N, O, and S; wherein the rings are optionally substituted with one or more substituents selected from halo, C1-C3 alkyl, 2-methoxyethyl or 2-(2′-methoxyethoxy-ethyl).

L₂ is absent or a single bond or is selected from 1,3,4-thiadiazole-2,5-diyl, —CONH—, —NHCO—, CONHCH₂—, —NH—, —NHCH(CF₃)—, —CON(CH₃)SO₂—, SO₂N(CH₃)CO—, —CCF₃—NH—; —SOCH₂— or —S(O)(NR₁₈)NH—; wherein R₁₈ is selected from C₁-C₆ alkyl, C₄-C₁₀ oxa-alkyl, C₄-C₁₀ dioxa-alkyl, or C₄-C₁₀ trioxa-alkyl.

Ring B is an optionally substituted 6-membered or 5-membered aryl, or heteroaryl.

X₅ and X₆ of Ring B are independently and separately selected from the group consisting of C or N.

In some embodiments, Ring B is an optionally substituted phenyl, pyridazine, pyridine, or thiophene.

R₆ attached to X₆ of Ring B is separately and independently H, F, Cl, Br, or I.

In some embodiments, when R₆ is attached to X₅ of Ring B, R₆ and L₁ taken together, along with the two intervening atoms to which they are attached, form an optionally substituted five-membered optionally substituted heterocycloalkyl ring having 2-3 heteroatoms independently selected from N, and S; wherein the rings are optionally substituted with one or more substituents selected from ═O, C₁-C₆ alkyl, or C₂-C₁₃ heteroalkyl, wherein the heteroatoms of the heteroalkyl are 1 to 3 oxygen atoms;

R₇ is CH═CH, CF═CH, N═CH, O or S;

Ring C is an optionally substituted 6-membered or 5-membered aryl, or heteroaryl; X₇ and X₈ of Ring C are independently and separately selected from the group consisting of C, or N;

In some embodiments, Ring C is optionally substituted phenyl, pyridazine, pyridine, thiophene. or furan,

R₉ attached to X₇ is H, or F

When R₉ is attached to X₈, then R₉, X₈ and L₃ taken together, along with intervening atoms to which they are attached, can form an optionally substituted five-membered heteroaromatic or heterocycloalkyl ring having 2-3 heteroatoms independently selected from N, O, and S; wherein the rings are optionally substituted with one or more substituents selected from halo, ═O, H, C₁-C₆ alkyl, C₂-C₁₁ heteroalkyl (with 1-3 oxygens).

R₈ is selected from CH═CH, CH═CF, C═N, S, or O;

L₃ is absent, a single bond, or selected from —CONH—, —NHCO—, CONHCH₂—, —NH—, —NHCH(CF₃)—, CON(CH₃)SO₂—, —SO₂N(CH₃)CO—, —CH₂SO—, —SOCH₂— and —CH(CF₃)—NH—CONHSO₂;

Ring D is an optionally substituted 6-membered aryl, or heteroaryl rings;

X₉ and X₁₀ of Ring D are independently and separately selected from the group consisting of C, or N;

R₁₃ of Ring D is —CH═N—, CH═CH, —N═C—, N═N, or S, all of which can be optionally substituted on carbon atoms except for S and N═N;

R₁₀, R₁₁, R₁₂ are independently and separately selected from the group consisting of H, F, C₁-C₆alkyl, CH₂COOH, CH(CH₃)COOH, COOH, SO₂NHCOR′, CONHSO₂R′, wherein each R′ is independently C₁-C₆ alkyl, C₂-C₆ heteroalkyl, 2-methoxyethyl, 2′-(2-methoxyethoxy)ethyl wherein each R′ can be optionally substituted with one or more of fluorine, C₁-C₄alkyl, C₁-C₄ heteroalkyl, and ═O, wherein exactly one of R₁₀, R₁₁, or R₁₂ is CH₂COOH or COOH, provided R₁₀ and R₁₁ do not combine to form a 6-membered ring.

R₁₀ and R₁₁ taken together, along with the two intervening atoms to which they are attached, form an optionally substituted five or six-membered aromatic, aliphatic heteroaromatic, or heteroaliphatic ring, so that ring D, R₁₀ and R₁₁ taken together form a bicyclic ring system, wherein the bicyclic ring system is substituted with exactly one substituent selected from COOH, SO₂NHCOR′, CONHSO₂R′, CH₂COOH, CH(CH₃)COOH, wherein each R′ is independently C₁-C₆ alkyl, C₂-C₆heteroalkyl, 2-methoxyethyl, or 2′-(2-methoxyethoxy)ethyl and wherein each R′ can be optionally substituted with one or more groups selected from fluorine, C₁-C₄alkyl, C₁-C₄ heteroalkyl, and ═O; and R₁₂ is H, F, or C₁-C₆alkyl.

Excluded from the disclosed and claimed compounds are compounds disclosed in publication no. WO 2017/106436.

In some embodiments, when Ring D and R₁₀ and R₁₁ taken together form naphthalene substituted with exactly one COOH, Ring A is phenyl, and R₁ is COOR′, wherein if R′ is C₁-C₅ alkyl then R₂ and R₃ are not H or COOR′.

In some embodiments, when Ring D and R₁₀ and R₁₁ taken together form naphthalene substituted with exactly one COOH, and Ring A is phenyl and one or more of R₁, R₂ and R₃ are COOR′, where R′ is C₁-C₅ alkyl, Rings B and C together are 3,3′-bipyridine.

In some embodiments, when Ring D and R₁₀ and R₁₁ taken together form naphthalene substituted with exactly one COOH, and Ring A is phenyl and one or more of R₁, R₂ and R₃ is COOR′, where R′ is C₁-C₅ alkyl one or both of Ring B and Ring C are pyridazine.

In some embodiments, when Ring D and R₁₀ and R₁₁ taken together form naphthalene substituted with exactly one COOH, and Ring A is phenyl and one or more of R₁, R₂ and R₃ are COOR′, where R′ is C₁-C₅ alkyl, Ring C and L₃ together are picolinamido.

In some embodiments, when Ring D and R₁₀ and R₁₁ form naphthalene and R₁₂ is carboxylic acid, Ring A is phenyl, L₁ is —CONH—, —NCO—, or SOCH₂, L₂ is absent, and L₃ is —CONH—, —NCO—, or —CH₂SO—.

In some embodiments, when Ring D and R₁₀ and R₁₁ form naphthalene and R₁₂ is carboxylic acid, Ring A is phenyl and L₁ is absent, and R₆ is independently selected from H, halogen or alkyl, X₇ is C or N

In some embodiments, when Ring D with R₁₀, R₁₁, and R₁₂ is a naphthalene-carboxylic acid, then R₁, R₂, and R₃ are not —COR₁₇, COOR₁₇, —NH₂, —Cl, —F, or —CF₃ where R₁₇ is C₁₋₅ alkyl.

In some embodiments, when Ring D is naphthalene, there is exactly one COOH substituent on the naphthalene ring.

In some embodiments, when Ring D is naphthalene, the phenyl ring formed by R₁₀ and R₁₁ is independently substituted with exactly one of the following substituents: COOH, SO₂NHR′, wherein R′ is CO(C₁-C₆ alkyl) or CO(C₈-heteroalklyl) in which 2 carbons are replaced with oxygen.

In some embodiments, when Ring D is naphthalene, the phenyl ring formed by R₁₀ and R₁₁ is independently substituted with one or more of COOH, SO₂NHR′, wherein R′ is CO(C₁-C₆ alkyl) or COC(C₈-heteroalklyl) (in which 2 carbons are replaced with oxygen), L₃ is —CONH—, —NCO—, CONHCH₂—, —NH—, —NHCH(CF₃)—, CONHSO₂—, or —CCF₃—NH—, Ring B is optionally substituted phenyl, pyridazine, pyridine, or thiophene and Ring C is optionally substituted phenyl, pyridazine, pyridine, thiophene, or furan, L₁ and L₂ are as described above, Ring A is optionally substituted phenyl, 1,3 4-thiadiazole, or piperidine.

In some embodiments, Ring D is naphthalene, substituted with a single COOH, Ring B is optionally substituted phenyl, pyridazine, pyridine, or thiophene and Ring C is optionally substituted phenyl, pyridazine, pyridine, thiophene, or furan, L₃ is —CONH—, —NCO—, CONHCH₂—, —NH—, —NHCH(CF₃)—, CONHSO₂—, or —CCF₃—NH—, L₁ and L₂ are as described above, Ring A is optionally substituted phenyl, 1,3,4-thiadiazole, piperidine.

In some embodiments, the compounds disclosed herein are selected from one or more of the following:

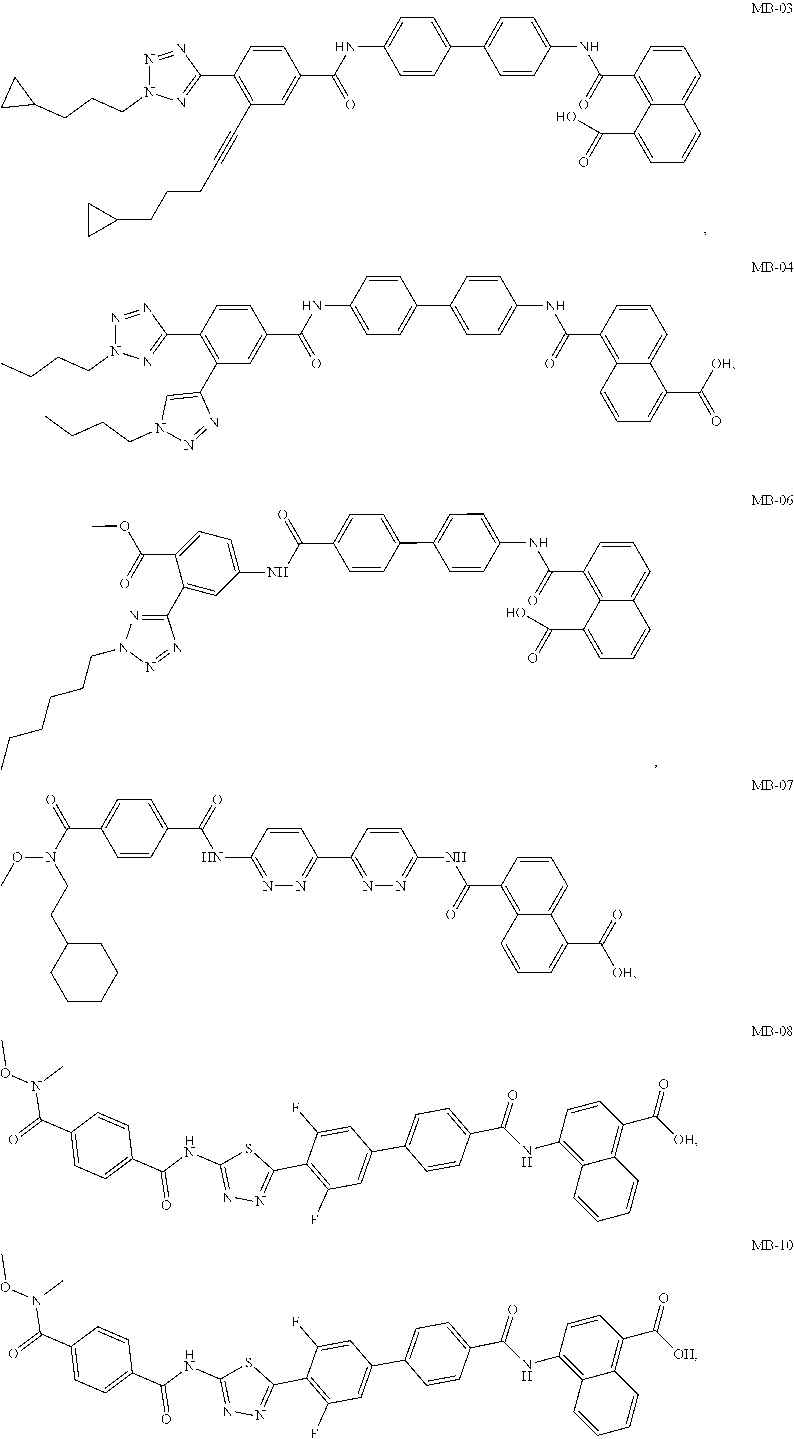

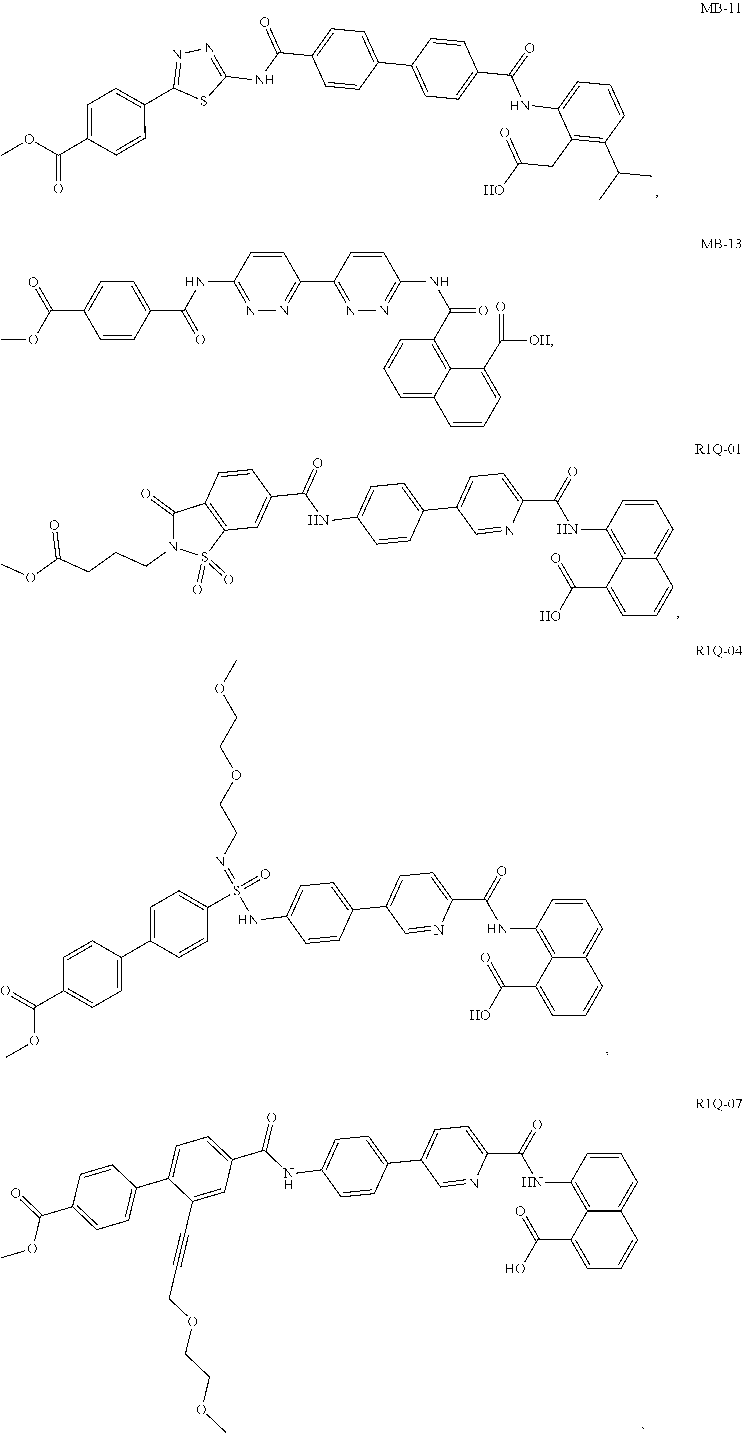

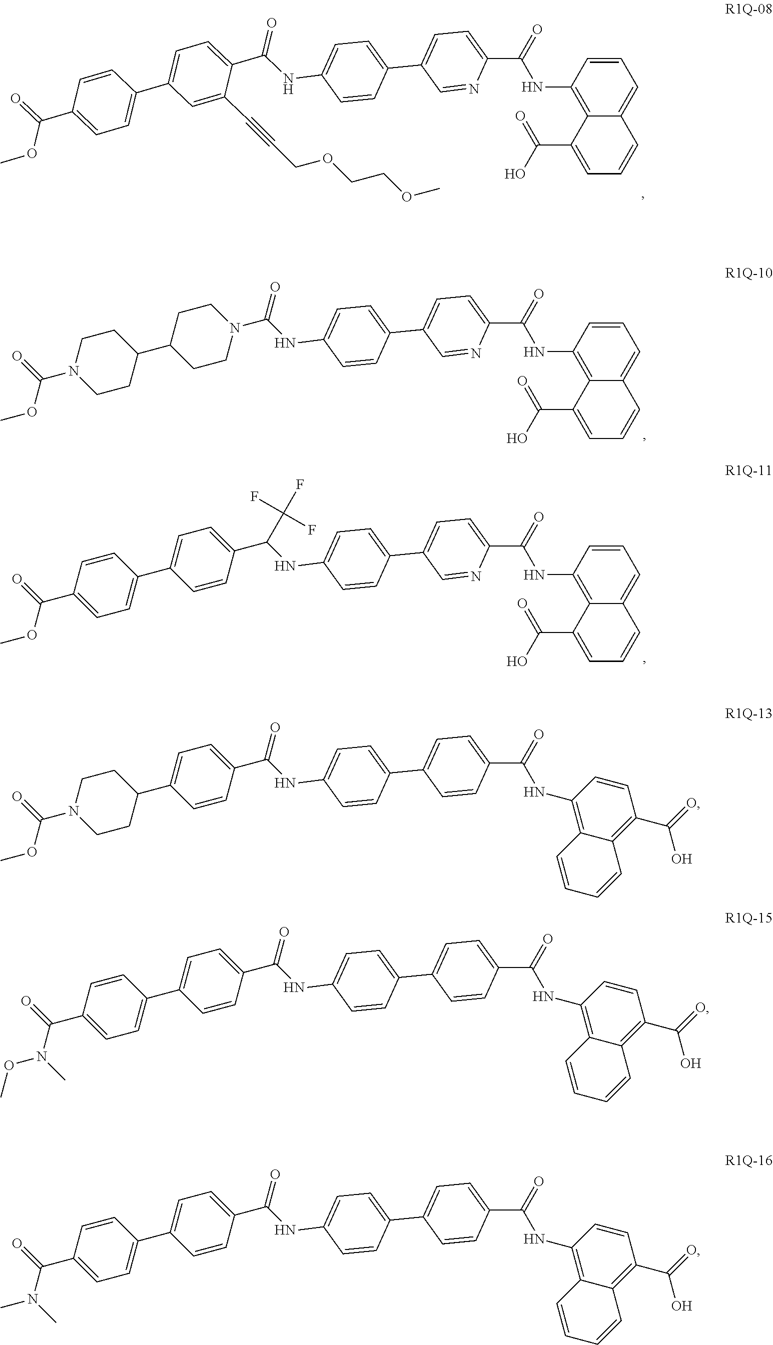

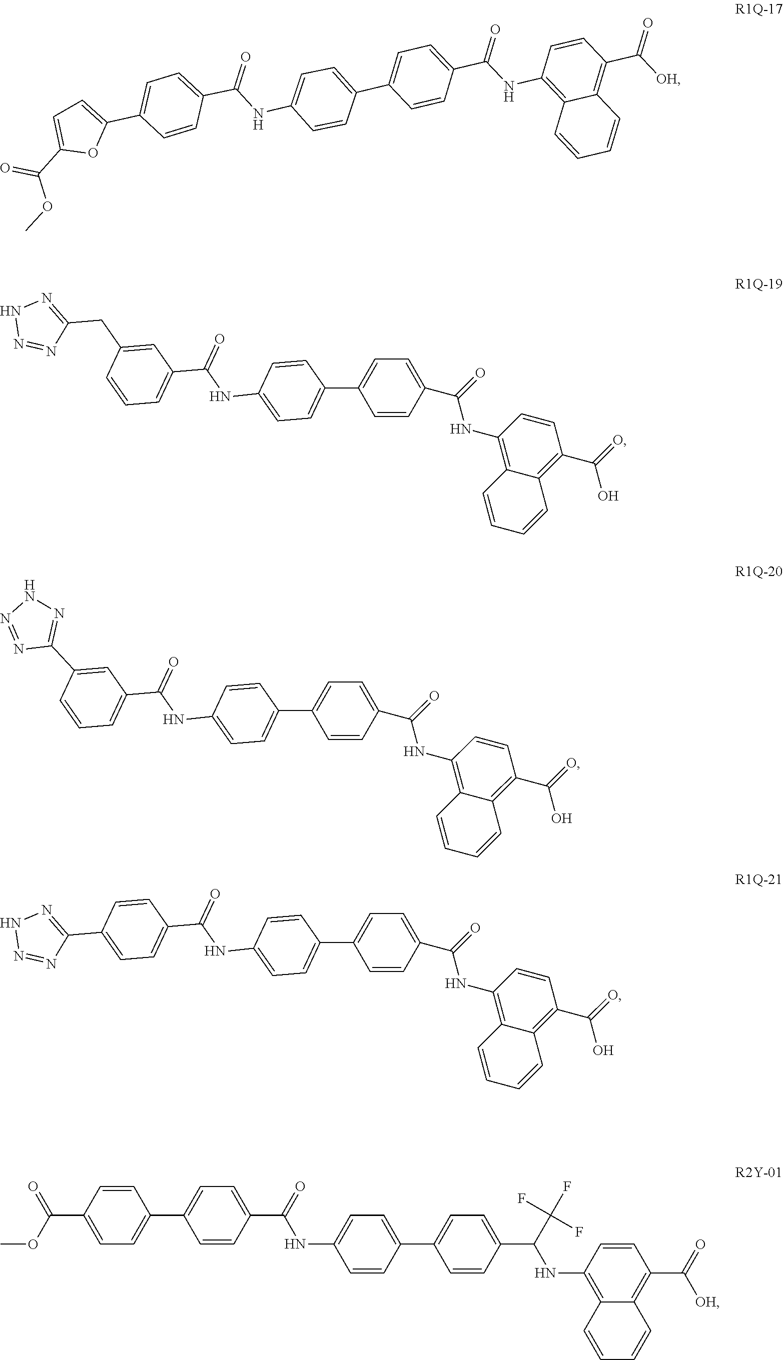 [SMILES: COC(=O)c1ccc(-c2ccc(C(=O)Nc3ccc(-c4ccc(C(=O)Nc5ccc(C(=O)O)c6ccccc56)cc4)cc3)cc2)o1 ; O=C(Nc1ccc(-c2ccc(C(=O)Nc3ccc(C(=O)O)c4ccccc34)cc2)cc1)c1cccc(Cc2nn[nH]n2)c1 ; O=C(Nc1ccc(-c2ccc(C(=O)Nc3ccc(C(=O)O)c4ccccc34)cc2)cc1)c1cccc(-c2nn[nH]n2)c1 ; O=C(Nc1ccc(-c2ccc(C(=O)Nc3ccc(C(=O)O)c4ccccc34)cc2)cc1)c1ccc(-c2nn[nH]n2)cc1 ; COC(=O)c1ccc(-c2ccc(C(=O)Nc3ccc(-c4ccc(C(Nc5ccc(C(=O)O)c6ccccc56)C(F)(F)F)cc4)cc3)cc2)cc1]

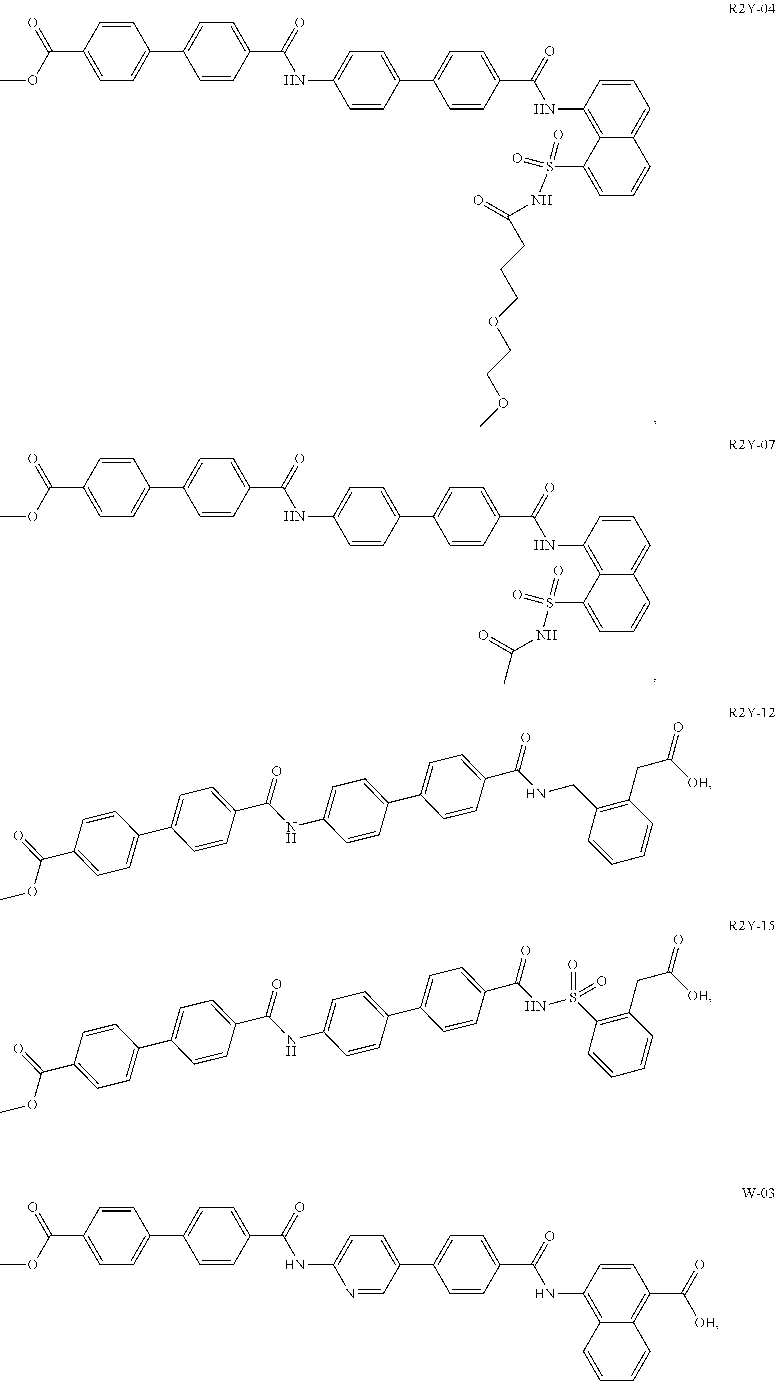 [SMILES: COCCOCCCC(=O)NS(=O)(=O)c1cccc2cccc(NC(=O)c3ccc(-c4ccc(NC(=O)c5ccc(-c6ccc(C(=O)OC)cc6)cc5)cc4)cc3)c12 ; COC(=O)c1ccc(-c2ccc(C(=O)Nc3ccc(-c4ccc(C(=O)Nc5cccc6cccc(S(=O)(=O)NC(C)=O)c56)cc4)cc3)cc2)cc1 ; COC(=O)c1ccc(-c2ccc(C(=O)Nc3ccc(-c4ccc(C(=O)NCc5ccccc5CC(=O)O)cc4)cc3)cc2)cc1 ; COC(=O)c1ccc(-c2ccc(C(=O)Nc3ccc(-c4ccc(C(=O)NS(=O)(=O)c5ccccc5CC(=O)O)cc4)cc3)cc2)cc1 ; COC(=O)c1ccc(-c2ccc(C(=O)Nc3ccc(-c4ccc(C(=O)Nc5ccc(C(=O)O)c6ccccc56)cc4)cn3)cc2)cc1]

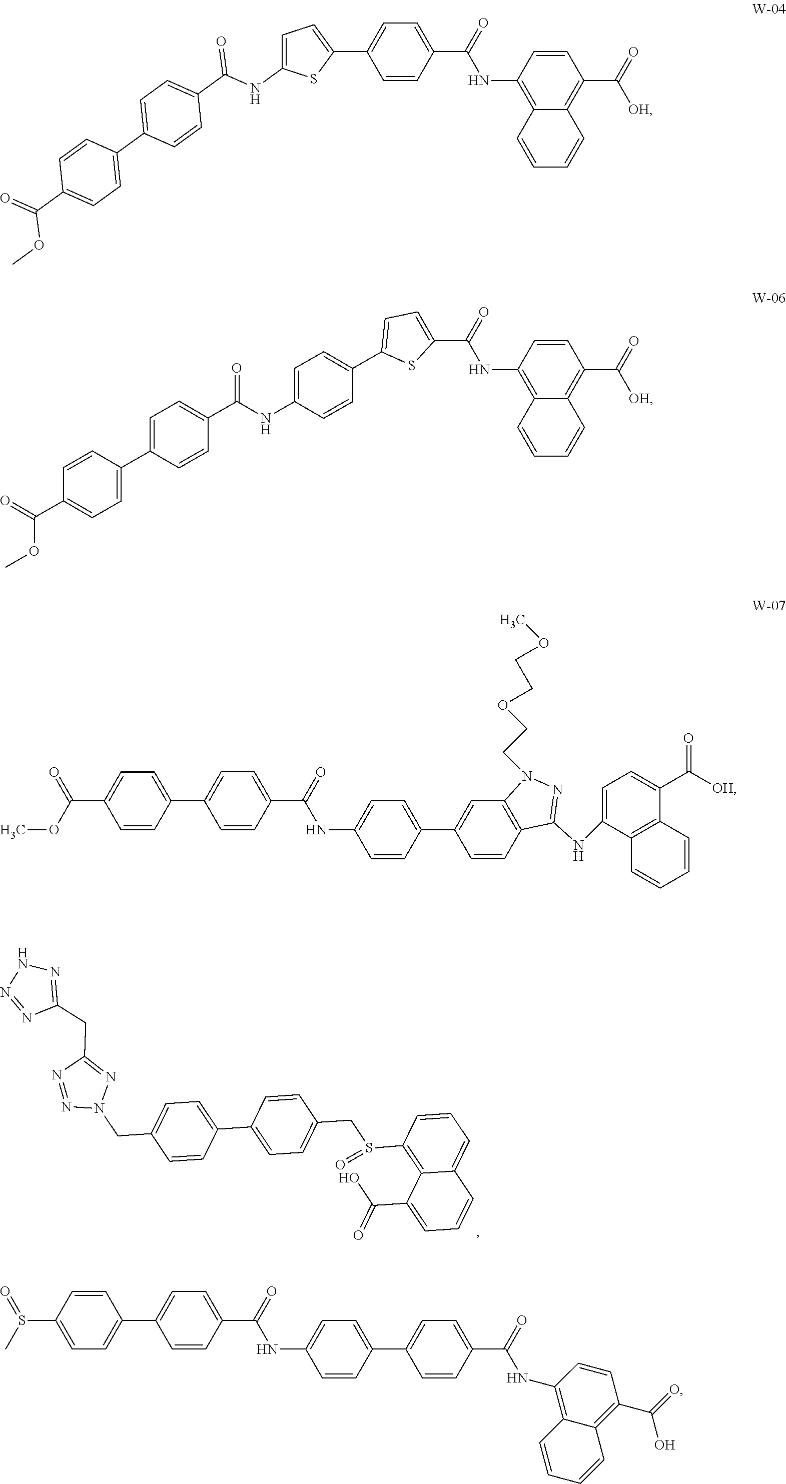 [SMILES: COC(=O)c1ccc(-c2ccc(C(=O)Nc3ccc(-c4ccc(C(=O)Nc5ccc(C(=O)O)c6ccccc56)cc4)s3)cc2)cc1 ; COC(=O)c1ccc(-c2ccc(C(=O)Nc3ccc(-c4ccc(C(=O)Nc5ccc(C(=O)O)c6ccccc56)s4)cc3)cc2)cc1 ; COCCOCCn1nc(Nc2ccc(C(=O)O)c3ccccc23)c2ccc(-c3ccc(NC(=O)c4ccc(-c5ccc(C(=O)OC)cc5)cc4)cc3)cc21 ; O=C(O)c1cccc2cccc(S(=O)Cc3ccc(-c4ccc(Cn5nnc(Cc6nn[nH]n6)n5)cc4)cc3)c12 ; CS(=O)c1ccc(-c2ccc(C(=O)Nc3ccc(-c4ccc(C(=O)Nc5ccc(C(=O)O)c6ccccc56)cc4)cc3)cc2)cc1]

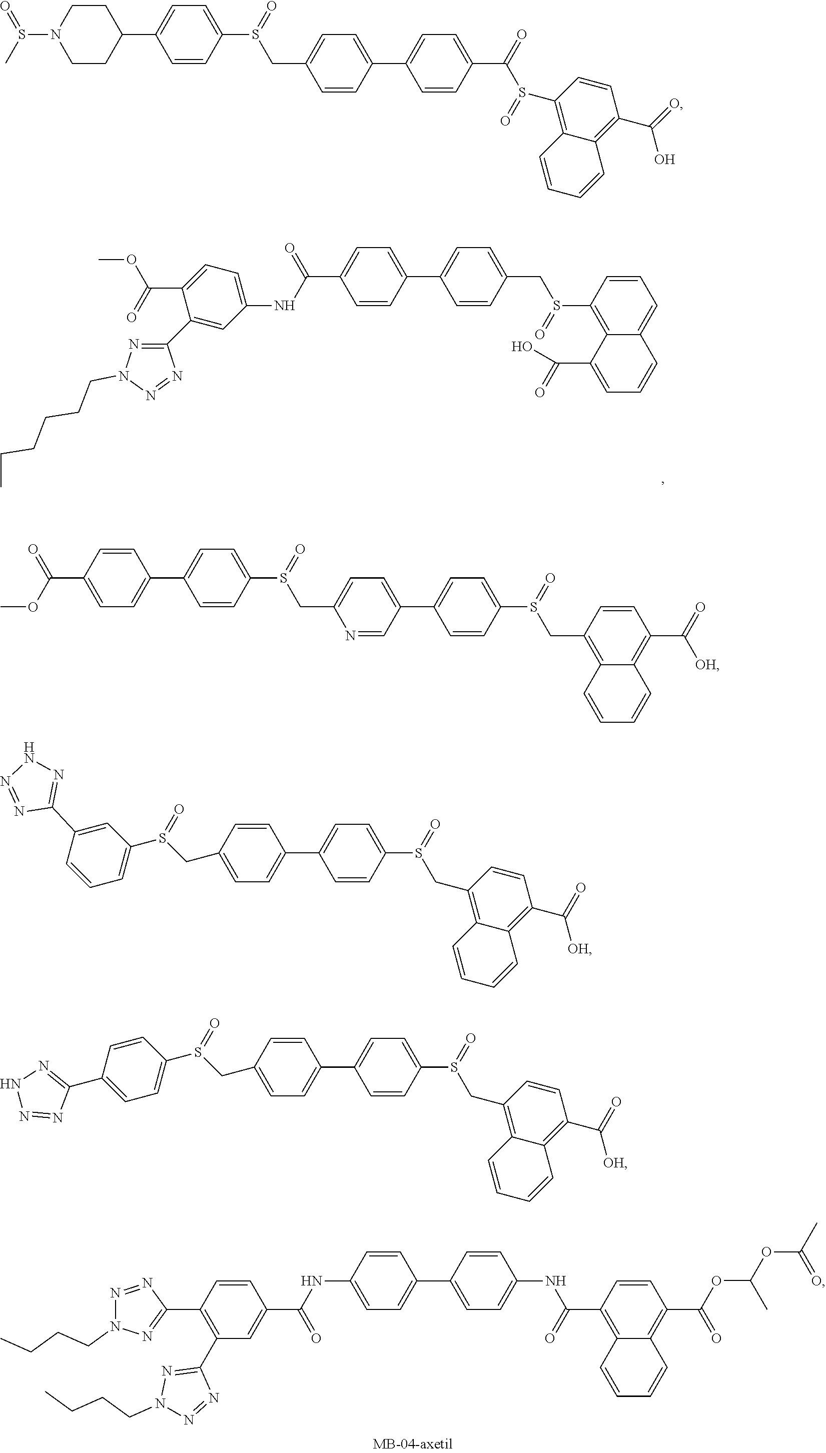

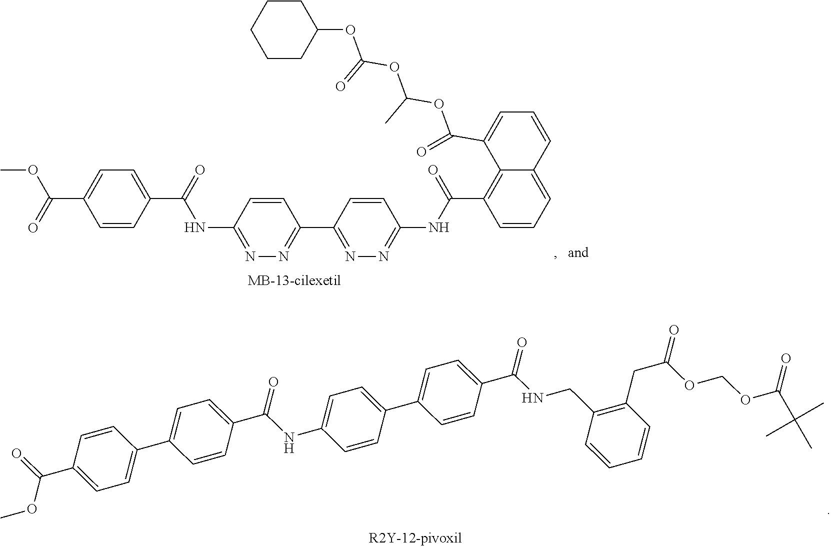 [SMILES: COC(=O)c1ccc(C(=O)Nc2ccc(-c3ccc(NC(=O)c4cccc5cccc(C(=O)OC(C)OC(=O)OC6CCCCC6)c45)nn3)nn2)cc1 ; COC(=O)c1ccc(-c2ccc(C(=O)Nc3ccc(-c4ccc(C(=O)NCc5ccccc5CC(=O)OCOC(=O)C(C)(C)C)cc4)cc3)cc2)cc1]

In some embodiments, a compound disclosed herein is selected from one or more of the following:

8-({4′-[3-(5-cyclopropylpent-1-yn-1-yl)-4-[2-(3-cyclopropylpropyl)-2H-
1,2,3,4-tetrazol-5-yl]benzamido]-[1,1′-biphenyl]-4-yl}carbamoyl)naphthalene-
1-carboxylic acid;
5-({4′-[3-(1-butyl-1H-1,2,3-triazol-4-yl)-4-(2-butyl-2H-1,2,3,4-tetrazol-5-
yl)benzamido]-[1,1′-biphenyl]-4-yl}carbamoyl)naphthalene-1-carboxylic acid;
8-[(4′-{[3-(2-hexyl-2H-1,2,3,4-tetrazol-5-yl)-
4(methoxycarbonyl)phenyl]carbamoyl}-[1,1′-biphenyl]-
4yl)carbamoyl]naphthalene-1-carboxylic acid;
5-[(6′-{4-[(2-cyclohexylethyl)(methoxy)carbamoyl]benzamido}-[3,3′-
bipyridazine]-6-yl)carbamoyl]naphthalene-1-carboxylic acid;
4-[3′,5′-difluoro-4′-(5-[4-[methoxy(methyl)carbamoyl]benzamido}-1,3,4-
thiadiazol-2-yl)-[1,1′-biphenyl]-4-amido]naphthalene-1-carboxylic acid;
5-[(6′-{4-[4-(2-ethoxy-2-oxoethyl)-1H-1,2,3-triazol-1-yl]benzamido}-[3,3′-
bipyridine]-6-yl)carbamoyl]naphthalene-1-carboxylic acid;
2-{2-[4′-({5-[4-(methoxycarbonyl)phenyl]-1,3,4-thiadiazol-2-yl}carbamoyl)-
[1,1′-biphenyl]-4-amido]-6-(propan-2-yl)phenyl}acetic acid;
8-({6′-[4-(methoxycarbonyl)benzamido]-[3,3′-bipyridazine]-6-
yl}carbamoyl)naphthalene-1-carboxylic acid;
8-(5-{4-[2-(4-methoxy-4-oxobutyl)-1,1,3-trioxo-2,3-dihydro-1lambda6,2-
benzothiazole-6-amido]phenyl}pyridine-2-amido)naphthalene-1-carboxylic
acid;
8-[5-(4-{N-[2-(2-methoxyethoxy)ethyl]4′-(methoxycarbonyl)-[1,1′-biphenyl]-4-
sulfonoimidamido}phenyl)pyridine-2-amido]naphthalene-1-carboxylic acid;
8-(5-{4-[4′-(methoxycarbonyl)-2-[3-(2-methoxyethoxy)prop-1-yn-1-yl]-[1,1′-
biphenyl]-4-amido]phenyl}pyridine-2-amido)naphthalene-1-carboxylic acid;
8-(5-{4-[4′-(methoxycarbonyl)-2′-[3-(2-methoxyethoxy)prop-1-yn-1-yl]-[1,1′-
biphenyl]-4-amido]phenyl}pyridine-2-amido)naphthalene-1-carboxylic acid;
8-[5-(4-{[1′-(methoxycarbonyl)-[4,4′-bipiperidine]-1-
carbonyl]amino}phenyl)pyridine-2-amido]naphthalene-1-carboxylic acid;
8-(5-(4-((2,2,2-trifluoro-1-(4′-(methoxycarbonyl)-[1,1′-biphenyl]-4-
yl)ethyl)amino)phenyl)picolinamido)-1-naphthoic acid;
4-(4′-{4-[1-(methoxycarbonyl)piperidin-4-yl]benzamido}-[1,1′-biphenyl]-4-
amido)naphthalene-1-carboxylic acid;
4-(4′-{4′-[methoxy(methyl)carbamoyl]-[1,1′-biphenyl]-4-amido}-[1,1′-
biphenyl]-4-amido)naphthalene-1-carboxylic acid;
4-{4′-[4′-(dimethylcarbamoyl)-[1,1′-biphenyl]-4-amido]-[1,1′-biphenyl]-4-
amido}naphthalene-1-carboxylic acid;
4-(4′-{4-[5-(methoxycarbonyl)furan-2-yl]benzamido}-[1,1′-biphenyl]-4-
amido)naphthalene-1-carboxylic acid;
4-(4′-{3-[(2H-1,2,3,4-tetrazol-5-yl)methyl]benzamido}-[1,1′-biphenyl]-4-
amido)naphthalene-1-carboxylic acid;
4-{4′-[3-(2H-1,2,3,4-tetrazol-5-yl)benzamido]-[1,1′-biphenyl]-4-
amido}naphthalene-1-carboxylic acid;
4-{4′-[4-(2H-1,2,3,4-tetrazol-5-yl)benzamido]-[1,1′-biphenyl]-4-
amido}naphthalene-1-carboxylic acid;
4-[(2,2,2-trifluoro-1-{4′-[4′-(methoxycarbonyl)-[1,1′-biphenyl]-4-amido]-[1,1′-
biphenyl]-4-yl}ethyl)amino]naphthalene-1-carboxylic acid;
methyl 4′-({4′-[(8-{[4-(2-methoxyethoxy)butanamido]sulfonyl}naphthalen-1-
yl)carbamoyl]-[1,1′-biphenyl]-4-yl}carbamoyl)-[1,1′-biphenyl]-4-carboxylate;
Methyl 4′-[(4′-{[8-(acetamidosulfonyl)naphthalen-1-yl]carbamoyl}-[1,1′-
biphenyl]-4-yl)carbamoyl]-[1,1′-biphenyl]-4-carboxylate;
2-{2-[({4′-[4′-(methoxycarbonyl)-[1,1′-biphenyl]-4-amido]-[1,1′-biphenyl]-4-
yl}formamido)methyl]phenyl}acetic acid;
2-{2-[({4′-[4′-(methoxycarbonyl)-[1,1′-biphenyl]-4-amido]-[1,1′-biphenyl]-4-
yl}formamido)sulfonyl]phenyl}acetic acid;
4-(4-{6-[4′-(methoxycarbonyl)-[1,1′-biphenyl]-4-amido]pyridin-3-
yl}benzamido)naphthalene-1-carboxylic acid;
4-(4-{5-[4′-(methoxycarbonyl)-[1,1′-biphenyl]-4-amido]thiophen-2-
yl}benzamido)naphthalene-1-carboxylic acid;
4-(5-{4-[4′-(methoxycarbonyl)-[1,1′-biphenyl]-4-amido]phenyl}thiophene-2-
amido)naphthalene-1-carboxylic acid;
4-{N-[2-(2-methoxyethoxy)ethyl]4′-[4′-(methoxycarbonyl)-[1,1′-biphenyl]-4-
amido]-[1,1′-biphenyl]-4-sulfonoimidamido}naphthalene-1-carboxylic acid;
8-(((4′-((5-((2H-tetrazol-5-yl)methyl)-2H-tetrazol-2-yl)methyl)-[1,1′-biphenyl]-
4-yl)methyl)sulfinyl)-1-naphthoic acid;
4-(4′-(4′-(methylsulfinyl)-[1,1′-biphenyl]-4-carboxamido)-[1,1′-biphenyl]-4-
carboxamido)-1-naphthoic acid;
4-(((4′-(((4-(1-(methoxycarbonyl)piperidin-4-yl)phenyl)sulfinyl)methyl)-[1,1′-
biphenyl]-4-yl)methyl)sulfinyl)-1-naphthoic acid;
4-(((4′-(((4-(1-(methoxycarbonyl)piperidin-4-yl)phenyl)sulfinyl)methyl)-[1,1′-
biphenyl]-4-yl)methyl)sulfinyl)-1-naphthoic acid;
4-(((4′-(((3-(2H-tetrazol-5-yl)phenyl)sulfinyl)methyl)-[1,1′-biphenyl]-4-
yl)sulfinyl)methyl)-1-naphthoic acid;
4-(((4′-(((4-(2H-tetrazol-5-yl)phenyl)sulfinyl)methyl)-[1,1′-biphenyl]-4-
yl)sulfinyl)methyl)-1-naphthoic acid; and
8-(((4′-((3-(2-hexyl-2H-tetrazol-5-yl)-4-(methoxycarbonyl)phenyl)carbamoyl)-
[1,1′-biphenyl]-4-yl)methyl)sulfinyl)-1-naphthoic acid.

In another aspect. disclosed herein are compounds of Formula II

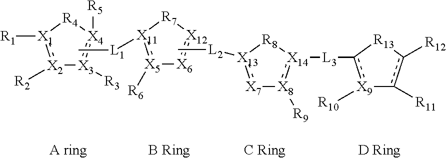

and pharmaceutically acceptable salts, esters, prodrugs, hydrates and tautomers thereof, wherein:

Rings A and D are optionally substituted 5- or 6-membered aromatic or heteroaromatic rings with 2-4 nitrogens, and rings B and C are optionally substituted 5- or 6-membered aromatic or heteroaromatic rings with 0-4 nitrogens.

In some embodiments of Formula II, X₁, X₂, X₃, X₄ are each separately and independently selected from the group consisting of C, or N.

In some embodiments of Formula II, R₄ is selected from CH═CH, S, O, N, N═CH, CH═N, N═N, or CH₂CH_{2;}

In some embodiments of Formula II, R₄ is N, X₁ is C, X₂, X₃ and X₄ are N;

In some embodiments of Formula II, R₄ is C═C, and X₁, X₂, X₃, X₄ are C;

In some embodiments of Formula II, R₄ is N, X₁ and X₂ are N, and X₃ and X₄ are C;

In some embodiments of Formula II, R₄ is N, X₁, X₂, X₃ are N, and X₄ is C;

In some embodiments of Formula II, R₄ is N, X₁ and X₄ are N, X₂ and X₃, are C;

In some embodiments, R₄ is C, X₁, X₂, X₃ are N, and X₄, is C;

In some embodiments of Formula II, Ring A is phenyl, benzene, pyridine, or triazole, or tetrazole.

In some embodiments, Ring A of Formula II may be optionally substituted with OH, SO₂NR′₂, SO₂R′, COR′, COOR′, CON(R′)₂, CON(OR′)R′, NCOR′, NO₂, tetrazole, triazol, alky-heteroaryl, or phenyl; wherein R′ is selected from C₁-C₅ alkyl, C₃-C₁₀ heteroalkyl wherein the heteroatoms are 1-3 oxygens, C₃-C₆ cycloalkyl, optionally substituted with 1-3 fluorine atoms;

In some embodiments the alkyl-heteroaryl is 5-ethyl-2H-tetrazole;

In some embodiments, the phenyl substituted on Ring A may be optionally substituted with OH, NHCOCH₃, SOCH₃, NHCH₃, COR′, COOR′, or CON(R′)₂, where R′ is independently selected from C₁-C₆ alkyl or C₁-C₃alkoxy.

L₁ is a bond, (CH₂)ₙ where (n=1-3), —NH—, 1,2,3-triazole linked at 1 and 4, or 5-alkyl-tetrazole linked at the 2 position and the alkyl terminus (alkyl is 0-3 carbons).

In some embodiments when L₁ is a bond, and rings A and B are fused to one another to form a heteroaromatic bicycle such as benzimidazole which can be optionally substituted;

In some embodiments the fused heteroaromatic ring is optionally substituted with SO₂R′ where R′ is a C₁-C₆alkyl.

In some embodiments the benzimidazole is substituted with SO₂R′ where R′ is a C₁-C₆alkyl.

Ring B is a diazole, triazole, tetrazole, pyridazine, pyrimidine, benzene, pyridine, piperidine, or piperazine.

L₂ is a bond, (CH₂)ₙ where n is 1 to 5, CH(OH), C(CH₃)₂, —CH(OH)—, —CH₂NH—, benzene-1,2-diyl, benzene-1,3-diyl, benzene-1,4-diyl, pyridine-3,5-diyl.

In some embodiments, when Ring B is a 6-membered ring, the relative positions of the L₁ and L₂ links to ring B can be 1,2; 1,3; or 1,4.

In some embodiments, when ring B is 1,2,3-triazole, L₁ is linked to the 1 position and L₂ is linked to the 4 position.

In some embodiments, if Ring B is a tetrazole, L₁ is linked to the 2 position and L₂ is linked to the 5 position.

In some embodiments, Ring B is imidazole.

Ring C is 1,2,3-triazole, tetrazole, benzene pyridine, pyridazine, 1,2,4-triazine, piperazine, or piperidine. The relative positions of the L₂ and L₃ links to ring C can be 1,2; 1,3; 3,5; 3,6; 2,5; or 1,4.

In some embodiments, L₃ is a bond, (CH₂)ₙ, (CH₂)ₙCO, —NHCO—, (CH₂)ₙCONH where n=0-3. If Ring C is a 6-membered ring, the relative positions of the L₂ and L₃ links to Ring C can be 1,2; 1,3; or 1,4.

In some embodiments, if Ring C is 1,2,3-triazole, L₃ is linked to the 1 position and L₂ is linked to the 4 position. If Ring C is a tetrazole, L₃ is linked to the 2 position and L₂ is linked to the 5 position.

Ring D is benzene, or pyridine, or thiophene.

In some embodiments, R₁₀ and R₁₁ of Ring D optionally form an aromatic ring fused to Ring D, including without limitation a fused benzene, or pyridine ring. In some embodiments, Ring D, R₁₀, and R₁₁ can form a bicyclic aromatic ring, including but not limited to naphthalene, quinoline, isoquinoline, or benzothiophene.

In some embodiments, when L₃ is a bond, rings C and D can optionally be fused to form a bicyclic ring such as quinoline, 1,2,3,4-tetrahydroquinoline, isoquinoline or naphthalene.

In some embodiments, R₄, L₁, Ring B, and Ring C contain at least 4 to 8 aromatic nitrogen atoms, with at least 1 pair of adjacent aromatic nitrogen atoms without substituents (N═N or N—NH).

In some embodiments, R₁ for Formula II is H, F, COOR₁₄, CONR₁₄, OR₁₅, SO₂R₁₄, SO₂NR₁₄, COR₁₅, tetrazole linked through its carbon, CH₂-tetrazole linked through its carbon.

R₁₄ and R₁₅ are, independently, C₁-C₁₀ alkyl, C₃-C₈ cycloalkyl, or C₃-C₆ cycloalkyl linked through 1-8 carbon alkyl chains {(CH₂)n with n=1 to 8}. The alkyls and cycloalkyls are optionally substituted with 1-3 fluorine atoms.

In some embodiments, R₂ of Formula II is H, F, COOR₁₄, CONR₁₄(OR₁₅), SO₂R₁₄, SO₂NR₁₄COR₁₅, tetrazole linked through its carbon, CH₂-tetrazole linked through its carbon; with the proviso that R₁ and R₂ cannot both be H or F;

In some embodiments, R₃ of Formula II is H, F or absent;

In some embodiments, R₄ Formula II is N, CH, or S (if ring A is a 5-membered aromatic rings), or R₄ is CH═CR₁₆ {where R₁₆ is OH, OCHF₂, NHCOR₁₄, H, F}, CH═N, or N═CH if ring A is a 6-membered aromatic ring.

In some embodiments, L₁ of Formula II can optionally combine with R₄ to form a heteroaromatic ring fused to ring A which can be optionally substituted with OH, SO₂NR′₂, SO₂R′, COR′, COOR′, CON(R′)₂, CON(OR′)R′, NHCOR′, tetrazole, triazole, and alkyl-heteroaryl, wherein R′ is selected from C₁-C₅ alkyl, C₃-C₁₀ heteroalkyl wherein the heteroatoms are 1-3 oxygens, C₃-C₆ cycloalkyl, optionally substituted with 1-3 fluorine atoms;

R₆ is H, F, methyl or absent;

R₇ is CH, N, CR6=CH, or N═CH, optionally substituted with methyl on carbon atoms;

R₈ is CH, N, CH═CH, CH═N, or N═CH, optionally substituted with methyl on carbon atoms;

R₉ is H, F, Cl, methyl or absent;

R₁₀ is H, CH₃, CH₂COOH, CH₂SO₂NHCOR₁₇, SO₂NHCOR₁₇, or tetrazole linked from its carbon (5 position);

R₁₁ is H, COOH, CH₂COOH, CH₂SO₂NHCOR₁₇, SO₂NHCOR₁₆, or tetrazole linked from its carbon (5 position);

R₁₀ and R₁₁ can optionally be linked to form an aromatic ring so that Ring D, R₁₀, and R₁₁ form a bicyclic aromatic ring such as naphthalene, isoquinoline, or benzthiophene optionally substituted with COOH, CH₂COOH, CH₂SO₂NHCOR₁₇, SO₂NHCOR₁₇, or tetrazole linked from its carbon (5 position);

R₁₂ is H or SO₂NHCOR₁₇;

R₁₃ is CH═CH, CH═C(COOH), CH═C(CH₂COOH), CH═C(SO₂NHCOR₁₇), CH═C(CH₂SO₂NHCOR₁₇);

R₁₇ is H, CH₃, N(CH₃)₂, (CH₂CH₂O)ₙCH₃, NCH₃((CH₂CH₂O)ₙCH₃, where n=1 to 6

R₁₃ can optionally be fused to another benzene, pyridine or thiophene ring so that Ring D and R₁₃ form a bicyclic aromatic ring such as such as naphthalene, isoquinoline, or benzthiophene.

In some embodiments, R₁₄ and R₁₅ are, independently, C₁-C₁₀ alkyl, C₃-C₈ cycloalkyl, or C₃-C₆ cycloalkyl linked through 1-8 carbon alkyl chains {(CH₂)ₙ with n=1 to 8}. The alkyls and cycloalkyls are optionally substituted with 1-3 fluorine atoms.

In some embodiments, R₁₆ is H, CH₃, N(CH₃)₂, (CH₂CH₂O)ₙCH₃, NCH₃((CH₂CH₂O)ₙCH₃, where n=1 to 6.

In some embodiments, R₁₇ is H, CH₃, N(CH₃)₂, (CH₂CH₂O)ₙCH₃, NCH₃((CH₂CH₂O)ₙCH₃, where n=1 to 6

In some embodiments, there will be exactly one acidic group ionizable to an anion at pH 7.4 in the drug form of the molecule (e.g. COOH, CH₂COOH, SO₂NHCOR₁₇, CH₂SO₂NHCOR₁₇, or tetrazole group). This acidic group will preferably be linked to ring D, R₁₀, R₁₁, R₁₂, or R₁₃. The acidic group in the drug form can optionally be administered as a neutral or cationic prodrug.

Optionally, the acidic group in the drug form of the molecule can be protected as a neutral or cationic prodrug (such as an ester) which is converted to the acid (monoanionic) form, optionally by proteases or other anions, in vivo.

In some embodiments, the compounds of Formula II are selected from one or more of the following:

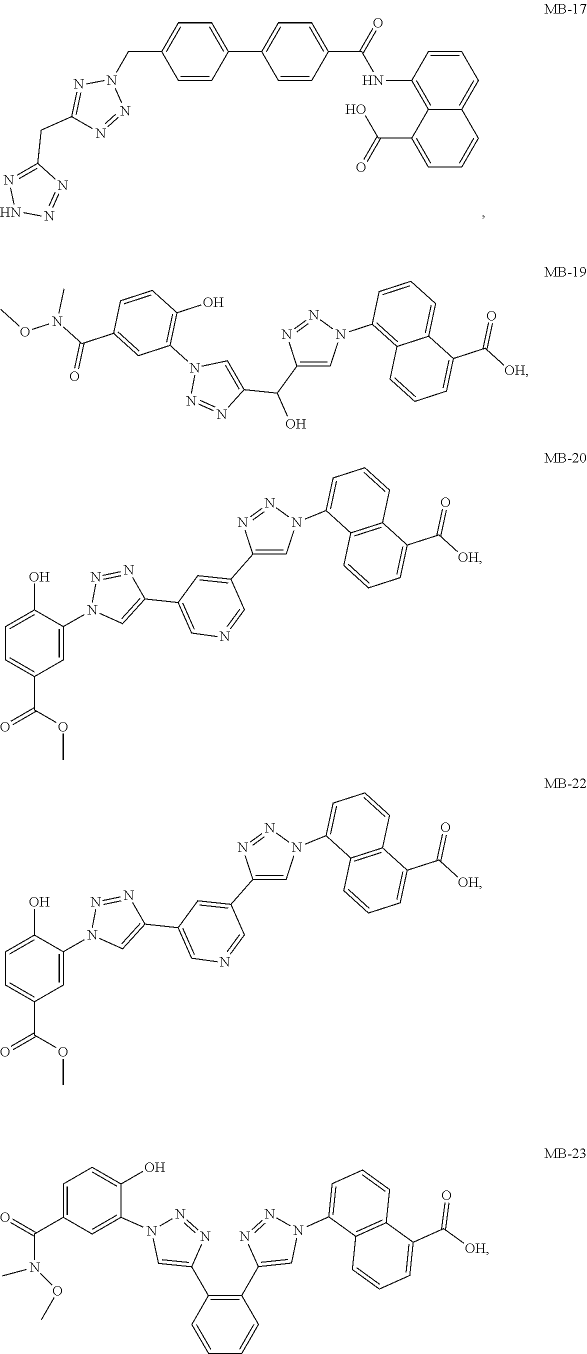 [SMILES: O=C(Nc1cccc2cccc(C(=O)O)c12)c1ccc(-c2ccc(Cn3nnc(Cc4nn[nH]n4)n3)cc2)cc1 ; CON(C)C(=O)c1ccc(O)c(-n2cc(C(O)c3cn(-c4cccc5c(C(=O)O)cccc45)nn3)nn2)c1 ; COC(=O)c1ccc(O)c(-n2cc(-c3cncc(-c4cn(-c5cccc6c(C(=O)O)cccc56)nn4)c3)nn2)c1 ; COC(=O)c1ccc(O)c(-n2cc(-c3cncc(-c4cn(-c5cccc6c(C(=O)O)cccc56)nn4)c3)nn2)c1 ; CON(C)C(=O)c1ccc(O)c(-n2cc(-c3ccccc3-c3cn(-c4cccc5c(C(=O)O)cccc45)nn3)nn2)c1]

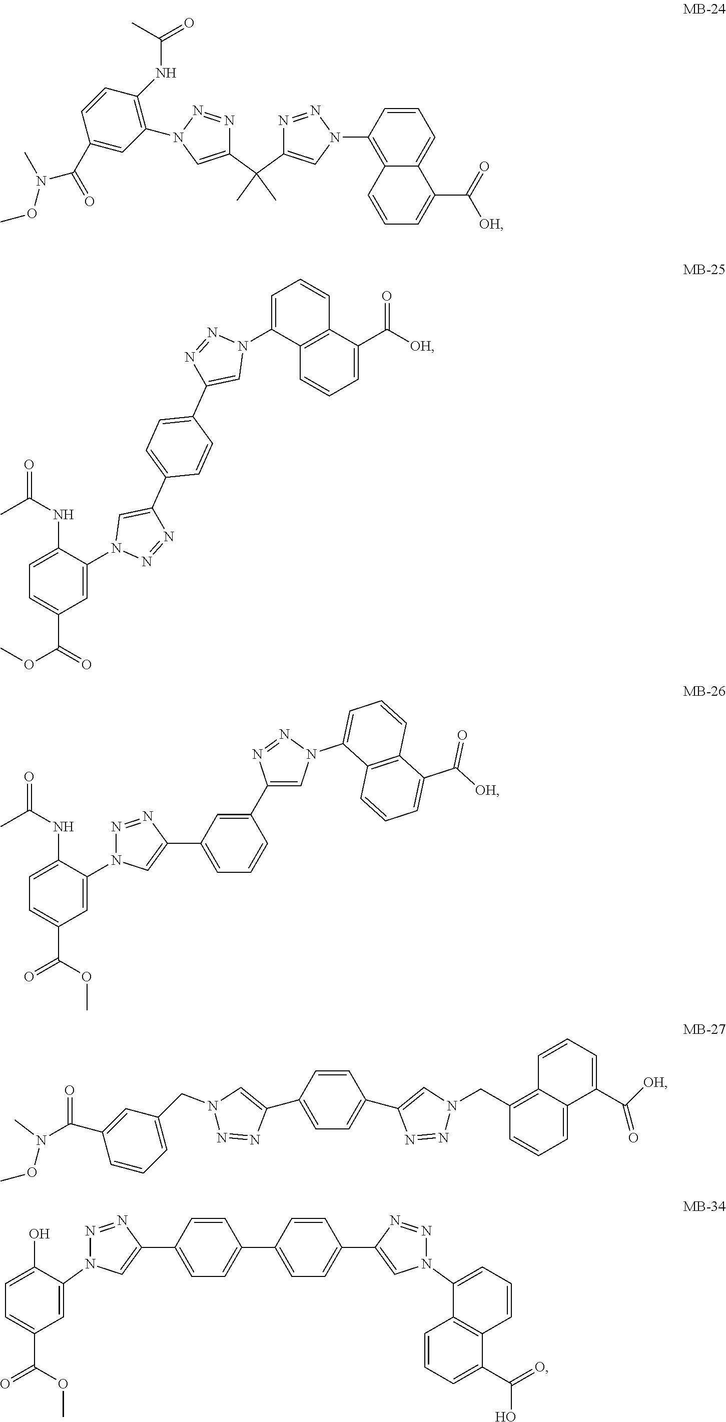 [SMILES: CON(C)C(=O)c1ccc(NC(C)=O)c(-n2cc(C(C)(C)c3cn(-c4cccc5c(C(=O)O)cccc45)nn3)nn2)c1 ; COC(=O)c1ccc(NC(C)=O)c(-n2cc(-c3ccc(-c4cn(-c5cccc6c(C(=O)O)cccc56)nn4)cc3)nn2)c1 ; COC(=O)c1ccc(NC(C)=O)c(-n2cc(-c3cccc(-c4cn(-c5cccc6c(C(=O)O)cccc56)nn4)c3)nn2)c1 ; CON(C)C(=O)c1cccc(Cn2cc(-c3ccc(-c4cn(Cc5cccc6c(C(=O)O)cccc56)nn4)cc3)nn2)c1 ; COC(=O)c1ccc(O)c(-n2cc(-c3ccc(-c4ccc(-c5cn(-c6cccc7c(C(=O)O)cccc67)nn5)cc4)cc3)nn2)c1]

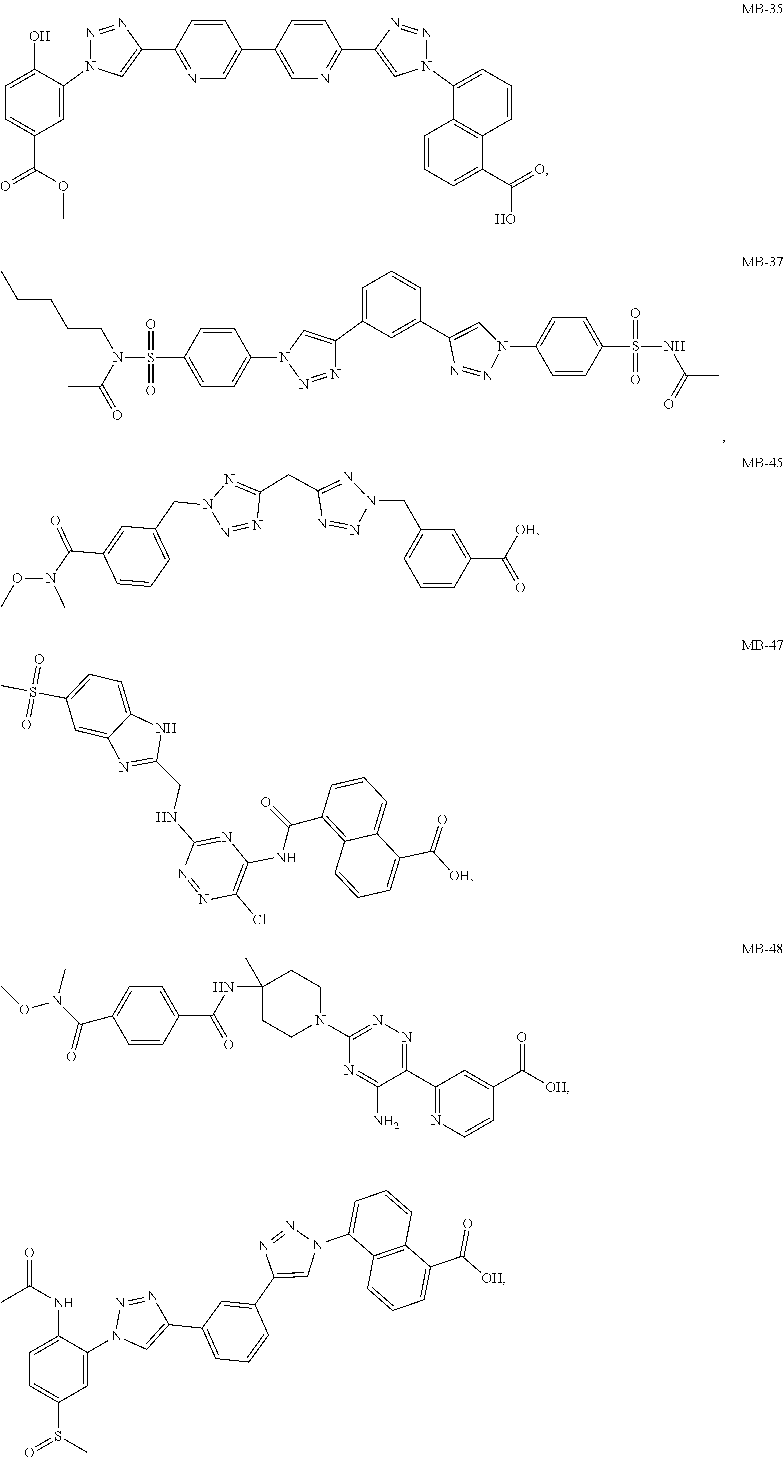

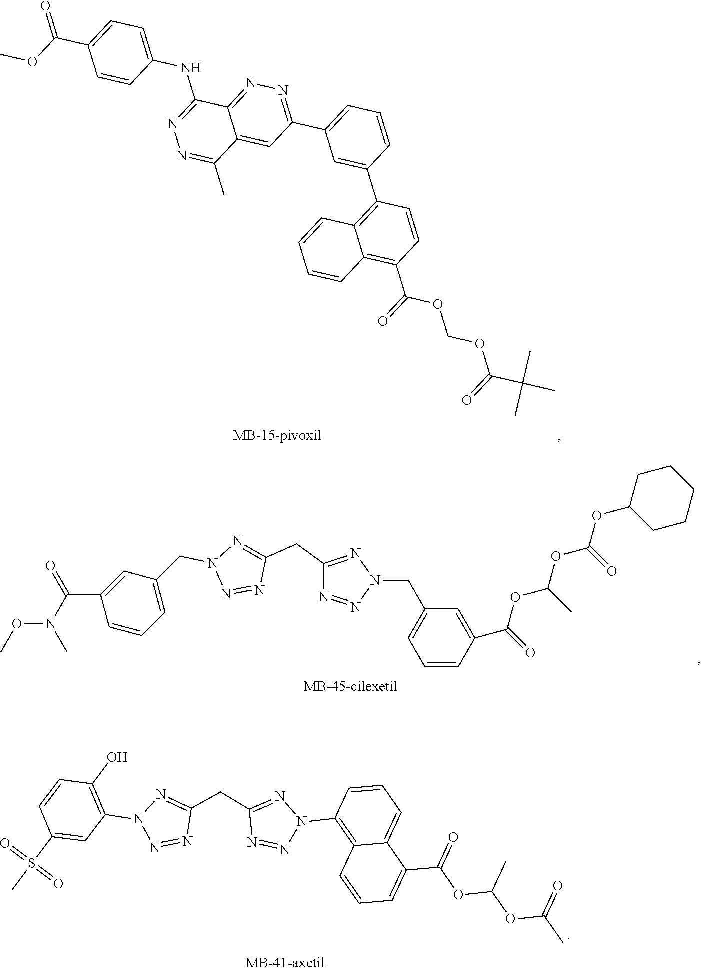

In some embodiments, the compound of Formula II is selected from one or more of the following:

8-[4′-({5-[(2H-1,2,3,4-tetrazol-5-yl)methyl]-2H-1,2,3,4-tetrazol-2-yl}methyl)-[1,1′-
biphenyl]-4-amido]naphthalene-1-carboxylic acid,
5-{4-[hydroxy(1-{2-hydroxy-5-[methoxy(methyl)carbamoyl]phenyl}-1H-1,2,3-triazol-4-
yl)methyl]-1H-1,2,3-triazol-1-yl}naphthalene-1-carboxylic acid,
5-[4-(5-{1-[2-hydroxy-5-(methoxycarbonyl)phenyl]-1H-1,2,3-triazol-4-yl}pyridin-3-yl)-
1H-1,2,3-triazol-1-yl]naphthalene-1-carboxylic acid,
3-{[5-({2-[(2-butanamido-5-nitrophenyl)methyl]-2H-1,2,3,4-tetrazol-5-yl}methyl)-2H-
1,2,3,4-tetrazol-2-yl]methyl}naphthalene-1-carboxylic acid,
5-{4-[2-(1-{2-hydroxy-5-[methoxy(methyl)carbamoyl]phenyl}-1H-1,2,3-triazol-4-
yl)phenyl]-1H-1,2,3-triazol-1-yl}naphthalene-1-carboxylic acid,
5-{4-[2-(1-{2-acetamido-5-[methoxy(methyl)carbamoyl]phenyl}-1H-1,2,3-triazol-4-
yl)propan-2-yl]-1H-1,2,3-triazol-1-yl}naphthalene-1-carboxylic acid,
5-[4-(4-{1-[2-acetamido-5-(methoxycarbonyl)phenyl]-1H-1,2,3-triazol-4-yl}phenyl)-1H-
1,2,3-triazol-1-yl]naphthalene-1-carboxylic acid,
8-[4′-({5-[(2H-1,2,3,4-tetrazol-5-yl)methyl]-2H-1,2,3,4-tetrazol-2-yl}methyl)-[1,1′-
biphenyl]-4-amido]naphthalene-1-carboxylic acid,
5-[4-(3-{1-[2-acetamido-5-(methoxycarbonyl)phenyl]-1H-1,2,3-triazol-4-yl}phenyl)-1H-
1,2,3-triazol-1-yl]naphthalene-1-carboxylic acid,
5-[(4-{4-[1-({3-[methoxy(methyl)carbamoyl]phenyl}methyl)-1H-1,2,3-triazol-4-
yl]phenyl}-1H-1,2,3-triazol-1-yl)methyl]naphthalene-1-carboxylic acid,
5-[4-(4′-{1-[2-hydroxy-5-(methoxycarbonyl)phenyl]-1H-1,2,3-triazol-4-yl}-[1,1′-
biphenyl]-4-yl)-1H-1,2,3-triazol-1-yl]naphthalene-1-carboxylic acid,
5-[4-(6′-{1-[2-hydroxy-5-(methoxycarbonyl)phenyl]-1H-1,2,3-triazol-4-yl}-[3,3′-
bipyridine]-6-yl)-1H-1,2,3-triazol-1-yl]naphthalene-1-carboxylic acid,
N-{4-[4-(3-{1-[4-(acetamidosulfonyl)phenyl]-1H-1,2,3-triazol-4-yl}phenyl)-1H-1,2,3-
triazol-1-yl]benzenesulfonyl}-N-pentylacetamide,
5-(4-(3-(1-(2-acetamido-5-(methylsulfinyl)phenyl)-1H-1,2,3-triazol-4-yl)phenyl)-1H-1,2,3-
triazol-1-yl)-1-naphthoic acid
3-[(5-{[2-({3-[methoxy(methyl)carbamoyl]phenyl}methyl)-2H-1,2,3,4-tetrazol-5-
yl]methyl}-2H-1,2,3,4-tetrazol-2-yl)methyl]benzoic acid,
5-[(6-chloro-3-{[(5-methanesulfonyl-1H-1,3-benzodiazol-2-yl)methyl]amino}-1,2,4-
triazin-5-yl)carbamoyl]naphthalene-1-carboxylic acid, and
2-[5-amino-3-(4-{4-[methoxy(methyl)carbamoyl]benzamido}-4-methylpiperidin-1-yl)-
1,2,4-triazin-6-yl]pyridine-4-carboxylic acid.

In another embodiment, the present disclosure provides a pharmaceutical composition comprising a compound of Formula III-11,11′-((2,5-bis(1H-benzo[d][1,2,3]triazol-1-yl)-3,6-dioxocyclohexa-1,4-diene-1,4-diyl)bis(azanediyl))diundecanoic acid, or a pharmaceutically acceptable salt thereof, in admixture with at least one pharmaceutically acceptable excipient. The synthesis of Formula III is described in Romanyuk et al., Russian Journal of General Chemistry (2006), 76(11):1834-1836.

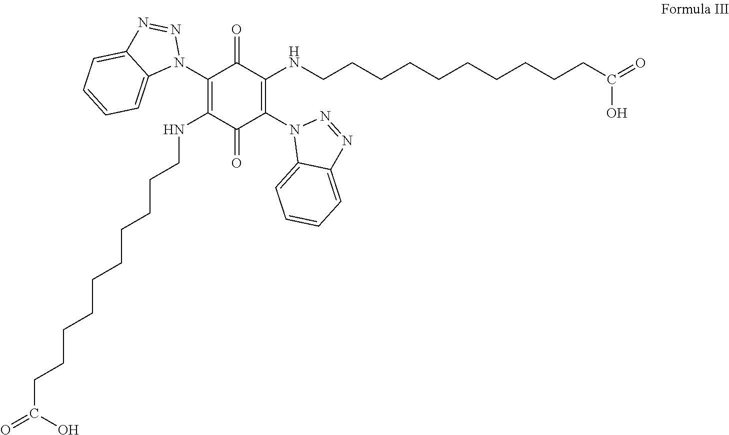 [SMILES: O=C(O)CCCCCCCCCCNC1=C(n2nnc3ccccc32)C(=O)C(NCCCCCCCCCCC(=O)O)=C(n2nnc3ccccc32)C1=O]

In another embodiment, the disclosure comprises the use of one or more compounds disclosed herein for the preparation of a medicament for the treatment of the conditions recited herein.

The compounds disclosed herein may be administered by any suitable route, preferably in the form of a pharmaceutical composition adapted to such a route, and in a dose effective for the treatment intended. The active compounds and compositions, for example, may be administered orally, rectally, parenterally, or topically (e.g., intranasal or ophthalmic).

Other carrier materials and modes of administration known in the pharmaceutical art may also be used. Pharmaceutical compositions disclosed herein may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

The compounds disclosed herein can be used, alone or in combination with other therapeutic agents, in the treatment of various conditions or disease states. The compound(s) disclosed herein and other therapeutic agent(s) may be administered simultaneously (either in the same dosage form or in separate dosage forms) or sequentially.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Treatment Indications

The compounds disclosed herein are useful for treating, ameliorating, or preventing an autoimmune disease, inflammatory disease, or other immune related disease, such as systemic lupus erythematosus (SLE), rheumatoid arthritis, ankylosing spondylitis, lupus nephritis, Goodpasture's disease, Sjögren's syndrome, polymyositis, dermatomyositis, psoriasis, temporal arteritis, Churg-Strauss syndrome, multiple sclerosis, Guillain-Barré syndrome, transverse myelitis, myasthenia gravis, Addison's disease, thyroiditis, coeliac disease, ulcerative colitis, Crohn's disease, sarcoidosis, hemolytic anemia, idiopathic thrombocytopenic purpura, Behçet's disease, primary biliary cirrhosis autoimmune diabetes, type 1 diabetes, Juvenile diabetes, angina pectoris, myocardial infarction, Alzheimer' s disease, traumatic brain injury, chronic traumatic encephalitis, Parkinson's disease, graft versus host disease, prevention and treatment of orthotopic and heterotopic solid organ transplants (e.g., without limitation, kidney, heart, liver, lung, cornea, pancreas, pancreatic islets, pancreatic islet-cells), xenotransplantation and transplants facilitated by pre-treatment/engraftment with donor bone marrow, and prevention and treatment of hematologic and solid organ malignancies comprising administering to a subject in need one or more compounds of the present disclosure.

In some embodiments, the compounds described herein are given in combination with other compounds, biologics, and other treatments known in the art and used in the treatment, amelioration, and prevention of the conditions and diseases discussed in paragraph in [00128].

In some embodiments, the compounds modulate the TNF superfamily costimulatory interactions.

In some embodiments, the compounds disclosed herein modulate one or more interactions of CD40-CD40L (CD154), TNF-R1-TNF-α, CD80(B7)-CD28, CD80(B7)-CD152(CTLA4), CD86(B7-2)-CD28, CD86-CD152, CD27-CD70, CD137(4-1BB)-4-1BBL, HVEM-LIGHT(CD258), CD30-CD30L, GITR-GITRL, BAFF-R(CD268)-BAFF(CD257), RANK(CD265)-RANKL(CD254), OX40(CD 134)-OX40L(CD252), and combinations thereof.

In some embodiments, the compounds described herein could be given before, concurrently, or after treatment with protein anti-CD154 agents.

In some embodiments, the compounds described herein are used to treat diseases and conditions associated with an inflammasome such as CNS Diseases, e.g., Alzheimer's disease, Parkinson's disease, multiple sclerosis, amyotrophic lateral sclerosis, stroke, acute brain trauma, and epilepsy (Swanton, T et al, SLAS Discovery, (2018) pgs. 1-27.)

In some embodiments, the compounds described herein are used to treat patients/subjects that have a high level of C-reactive protein, as determined by a medical professional such as a doctor, to treat, ameliorate and or prevent a cardiovascular event (Ridker, P. M., et al., (2018) Lancet 391:319-28).

In some embodiments, the compounds described herein are used to prevent transplant rejection (Langan M., et al., Nature (2018) December; 564(7736):430-433). In some embodiments the compounds are given before, concurrently or after administration of immunosuppressants used to prevent rejection of transplants such as without limitation steroids, mTor inhibitors, calcineurin inhibitors.

For the treatment of the conditions referred to above, the compounds disclosed herein can be administered as compound per se.

Alternatively, pharmaceutically acceptable salts are suitable for medical applications because of their greater aqueous solubility relative to the parent compound.

In another embodiment, the present disclosure comprises pharmaceutical compositions. Such pharmaceutical compositions comprise a compound disclosed herein presented with a pharmaceutically acceptable carrier. The carrier can be a solid, a liquid, or both, and may be formulated with the compound as a unit-dose composition, for example, a tablet, which can contain from 0.05% to 95% by weight of the active compounds. A compound disclosed herein may be coupled with suitable polymers as targetable drug carriers. Other pharmacologically active substances can also be present.

### Formulations

In another embodiment, the present disclosure comprises the use of one or more compounds disclosed herein for the preparation of a medicament for the treatment of the conditions recited herein.

The compounds disclosed herein may be administered orally. Oral administration may involve swallowing, so that the compound enters the gastrointestinal tract, or buccal or sublingual administration may be employed, by which the compound enters the blood stream directly from the mouth.

Oral administration of a solid dose form may be, for example, presented in discrete units, such as hard or soft capsules, pills, cachets, lozenges, or tablets, each containing a predetermined amount of at least one compound of the present disclosure. In another embodiment, the oral administration may be in a powder or granule form. In another embodiment, the oral dose form is sub-lingual, such as, for example, a lozenge. In such solid dosage forms, the compounds of the present disclosure are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the compounds of the disclosure may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the present disclosure comprises a parenteral dose form. “Parenteral administration” includes, for example, subcutaneous injections, intravenous injections, intraperitoneal injections, intramuscular injections, intracisternal injections, and infusion. Injectable preparations (i.e., sterile injectable aqueous or oleaginous suspensions) may be formulated according to the known art using suitable dispersing, wetting, and/or suspending agents, and include depot formulations.

In another embodiment, the compounds disclosed herein may also be formulated as a topical dose form such that administration topically to the skin or mucosa (i.e., dermally or transdermally) leads to systemic absorption of the compound. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Formulations suitable for topical administration to the eye include, for example, eye drops wherein the compound of this disclosure is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone; as a mixture, for example, in a dry blend with lactose; or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present disclosure comprises a rectal dose form. Such rectal dose form may be in the form of, for example, a suppository. Cocoa butter is a traditional suppository base, but various alternatives may be used as appropriate.

In another embodiment, the compounds of the disclosure may be formulated such that administration vaginally leads to systemic absorption of the compound.

The dosage regimen for the compounds and/or compositions containing the compounds is based on a variety of factors, including the type, age, weight, sex and medical condition of the patient; the severity of the condition; the route of administration; and the activity of the particular compound employed. Thus, the dosage regimen may vary widely. Dosage levels of the order from about 0.01 mg to about 100 mg per kilogram of body weight per day are useful in the treatment of the above-indicated conditions. In one embodiment, the total daily dose of a compound disclosed herein (administered in single or divided doses) is typically from about 0.01 to about 100 mg/kg. In another embodiment, the total daily dose of a compound disclosed herein is from about 0.1 to about 50 mg/kg, and in another embodiment, from about 0.5 to about 30 mg/kg (i.e., mg compound of the disclosure per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Oral administration of a solid dose form may be, for example, presented in discrete units, such as hard or soft capsules, pills, cachets, lozenges, or tablets, each containing a predetermined amount of at least one compound of the present disclosure. In another embodiment, the oral administration may be in a powder or granule form. In another embodiment, the oral dose form is sub-lingual, such as, for example, a lozenge. In such solid dosage forms, the compounds of the present disclosure are ordinarily combined with one or more adjuvants. Such capsules or tablets may contain a controlled-release formulation. In the case of capsules, tablets, and pills, the dosage forms also may comprise buffering agents or may be prepared with enteric coatings.

Suitable subjects/patients according to the present disclosure include mammalian subjects. Mammals according to the present disclosure include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### DEFINITIONS AND EXAMPLES

As used throughout this application, including the claims, the following terms have the meanings defined below, unless specifically indicated otherwise. The plural and singular should be treated as interchangeable, other than the indication of number: As used herein, the term “n-membered” where n is an integer typically describes the number of ring-forming atoms in a moiety where the number of ring-forming atoms is n. For example, pyridine is an example of a 6-membered heteroaryl ring and thiazole is an example of a 5-membered heteroaryl group.

At various places in the present specification, substituents of compounds disclosed herein are disclosed in groups or in ranges. It is specifically intended that the disclosure include each and every individual subcombination of the members of such groups and ranges. For example, the term “(C₁-C₆)alkyl” is specifically intended to include C₁alkyl(methyl), C₂alkyl(ethyl), C₃ alkyl(propyl), C₄alkyl(butyl), C₅ alkyl(pentyl), and C₆alkyl (hexyl). For another example, the term “a (5- to 10-membered) heterocycloalkyl group” is specifically intended to include any 5-, 6-, 7-, 8-, 9-, and 10-membered heterocycloalkyl group.

As used herein, “aryl” refers to a carbocyclic (all carbon) ring that has a fully delocalized pi-electron system. The “aryl” group can be made up of two or more fused rings (rings that share two adjacent carbon atoms). When the aryl is fused ring system, then the ring that is connected to the rest of the molecule has a fully delocalized pi-electron system. The other ring(s) in the fused ring system may or may not have a fully delocalized pi-electron system. Examples of aryl groups include, without limitation, benzene, naphthalene, and azulene.

As used herein, “heteroaryl” refers to a ring that has a fully delocalized pi-electron system and contains one or more heteroatoms selected from the group consisting of nitrogen, oxygen and sulfur, in the ring. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, phthalazinone, pyrrole, oxazole, thiazole, imidazole, pyrazole, isoxazole, isothiazole, triazole, thiadiazole, pyran, pyridine, pyridazine, pyrimidine, pyrazine, pyridazino[4,5-c]pyridazine and triazine.

As used herein, “alkyl” refers to a straight or branched chain fully saturated (no double or triple bonds) hydrocarbon group. An alkyl group of this disclosure may comprise from 1 to 20 carbon atoms. An alkyl group herein may also be of medium size having 1 to 10 carbon atoms. An alkyl group herein may also be a lower alkyl having 1 to 6 carbon atoms, i.e., (C₁-C₆)alkyl. Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl and dodecyl.

An alkyl group of this disclosure may be substituted or unsubstituted. When substituted, the substituent group(s) can be one or more group(s) independently selected from cycloalkyl, aryl, heteroaryl, heteroalicyclyl, hydroxy, protected hydroxyl, alkoxy, aryloxy, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, —NR^{a}R^{b} and protected amino

As used herein, “alkenyl” refers to an alkyl group that contains in the straight or branched hydrocarbon chain one or more double bonds. An alkenyl group of this disclosure may be unsubstituted or substituted. When substituted, the substituent(s) may be selected from the same groups disclosed above with regard to alkyl group substitution, or with regard to optional substitution.

As used herein, “alkynyl” refers to an alkyl group that contains in the straight or branched hydrocarbon chain one or more triple bonds. An alkynyl group of this disclosure may be unsubstituted or substituted. When substituted, the substituent(s) may be selected from the same groups disclosed above with regard to alkyl group substitution, or with regard to optional substitution.

The term “(C₁-C₆)alkoxy” as used herein, refers to a (C₁-C₆)alkyl group, as defined above, attached to the parent molecular moiety through an oxygen atom. Representative examples of a (C₁-C₆)alkoxy include, but are not limited to, methoxy, ethoxy, propoxy, 2-propoxy, butoxy, tert-butoxy, pentyloxy, and hexyloxy.

As used herein, “acyl” refers to an “RC(═O)—” group with R as defined above.

As used herein, “cycloalkyl” refers to a completely saturated (no double bonds) hydrocarbon ring. Cycloalkyl groups of this disclosure may range from C₃ to C₈. A cycloalkyl group may be unsubstituted or substituted. If substituted, the substituent(s) may be selected from those indicated above with regard to substitution of an alkyl group. The “cycloalkyl” group can be made up of two or more fused rings (rings that share two adjacent carbon atoms). When the cycloalkyl is a fused ring system, then the ring that is connected to the rest of the molecule is a cycloalkyl as defined above. The other ring(s) in the fused ring system may be a cycloalkyl, a cycloalkenyl, an aryl, a heteroaryl, or a heteroalicyclic.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of this disclosure may unsubstituted or substituted. When substituted, the substituent(s) may be selected from the same groups disclosed above with regard to alkyl group substitution. The “cycloalkenyl” group can be made up of two or more fused rings (rings that share two adjacent carbon atoms). When the cycloalkenyl is a fused ring system, then the ring that is connected to the rest of the molecule is a cycloalkenyl as defined above. The other ring(s) in the fused ring system may be a cycloalkyl, a cycloalkenyl, an aryl, a heteroaryl, or a heteroalicyclic.

The term “alkylene” refers to an alkyl group, as defined here, which is a biradical and is connected to two other moieties. Thus, methylene (—CH₂—), ethylene (—CH₂CH₂—), proylene (—CH₂CH₂CH₂—), isopropylene (—CH₂—CH(CH₃)—), and isobutylene (—CH₂—CH(CH₃)—CH₂—) are examples, without limitation, of an alkylene group. Similarly, the term “cycloalkylene” refers to an cycloalkyl group, as defined herein, which binds in an analogous way to two other moieties. If the alkyl and cycloalkyl groups contain unsaturated carbons, the terms “alkenylene” and “cycloalkenylene” are used.

As used herein, “heterocycloalkyl,” “heteroalicyclic” or heteroalicyclyl” refers to a ring or one or more fused rings having in the ring system one or more heteroatoms independently selected from nitrogen, oxygen and sulfur. The rings may also contain one or more double bonds provided that they do not form a fully delocalized pi-electron system in all the rings. Heteroalicyclyl groups of this disclosure may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethanesulfonamido.

Heteroalkyl” refers to a straight- or branched-chain alkyl group preferably having from 2 to 14 carbons, more preferably 2 to 10 carbons in the chain, one or more of which has been replaced by a heteroatom selected from S, O, P and N. Exemplary heteroalkyls include alkyl ethers, secondary and tertiary alkyl amines, amides, alkyl sulfides, and the like. The group may be a terminal group or a bridging group. As used herein reference to the normal chain when used in the context of a bridging group refers to the direct chain of atoms linking the two terminal positions of the bridging group.

“halo” or “halogen”, as used herein, refers to a chlorine, fluorine, bromine, or iodine atom.

“hydroxy” or “hydroxyl”, as used herein, means an OH group.

“oxo”, as used herein, means a ═O moiety. When an oxo is substituted on a carbon atom, they together form a carbonyl moiety [—C(═O)—]. When an oxo is substituted on a sulfur atom, they together form a sulfoxide moiety [—S(═O)—]; when two oxo groups are substituted on a sulfur atom, they together form a sulfonyl moiety [—S(═O)₂—].

“Optionally substituted”, as used herein, means that substitution is optional and therefore includes both unsubstituted and substituted atoms and moieties. A “substituted” atom or moiety indicates that any hydrogen on the designated atom or moiety can be replaced with a selection from the indicated substituent group (up to and including that every hydrogen atom on the designated atom or moiety is replaced with a selection from the indicated substituent group), provided that the normal valency of the designated atom or moiety is not exceeded, and that the substitution results in a stable compound. For example, if a methyl group (i.e., —CH₃) is optionally substituted, then up to 3 hydrogen atoms on the carbon atom can be replaced with substituent groups.

The embodiments disclosed herein are also meant to encompass all pharmaceutically acceptable compounds of Formula (I), Formula (II), and Formula III, including isotopically-labeled compounds in which one or more atoms can be replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ⁿC, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I. These radiolabeled compounds could be useful to help determine or measure the effectiveness of the compounds, by characterizing, for example, the site or mode of action, or binding affinity to pharmacologically important site of action. Certain isotopically-labeled compounds of Formulas (I), (II), or (III) for example, those incorporating a radioactive isotope, may be useful in drug and/or substrate tissue distribution studies. The radioactive isotopes tritium, i.e. ³H, and carbon-14, i.e. ¹⁴C, may particularly be useful for this purpose in view of their ease of incorporation and ready means of detection.

Substitution with heavier isotopes such as deuterium, i.e. ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as C, F, O and N, can be useful in Positron Emission Topography (PET) studies for examining substrate receptor occupancy. Isotopically-labeled compounds of Formulas (I), (II), and (III) can generally be prepared by conventional techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include compounds (e.g., compounds of Formula (I), Formula (II), and Formula (III) (or pharmaceutically acceptable salts, prodrugs, or solvates thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of compounds or pharmaceutically acceptable salts, prodrugs, or solvates thereof, when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example, by employing starting materials in which one or more hydrogen atoms have been replaced by deuterium.

The embodiments disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments disclosed herein include compounds produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples. “Stable compound” and “stable structure” are meant to indicate a compound that is sufficiently robust to survive isolation to a useful degree of purity from a reaction mixture, and formulation into an efficacious therapeutic agent. “Mammal” includes humans and both domestic animals such as laboratory animals and household pets (e.g., cats, dogs, swine, cattle, sheep, goats, horses, rabbits), and non-domestic animals such as wildlife and the like. “Optional” or “optionally” means that the subsequently described event of circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

Examples of “pharmaceutically acceptable salts” of the compounds disclosed herein include salts derived from an appropriate base, such as an alkali metal (for example, sodium), an alkaline earth metal (for example, magnesium), ammonium and NX₄⁺ (wherein X is C₁-C₄ alkyl). Pharmaceutically acceptable salts of a nitrogen atom or an amino group include for example salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, hydrobromic, sulfuric, phosphoric and sulfamic acids. Pharmaceutically acceptable salts of a compound of a hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds disclosed herein will typically be pharmaceutically acceptable, i.e. they will be salts derived from a physiologically acceptable acid or base. However, salts of acids or bases which are not pharmaceutically acceptable may also find use, for example, in the preparation or purification of a compound of Formulas (I), (II), (III) or another compound of the embodiments disclosed herein. All salts, whether or not derived from a physiologically acceptable acid or base, are within the scope of the embodiments disclosed herein.

Metal salts typically are prepared by reacting the metal hydroxide with a compound according to the embodiments disclosed herein. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines Finally, it is to be understood that the compositions herein comprise compounds disclosed herein in their un-ionized, as well as zwitterionic form, and combinations with stoichiometric amounts of water as in hydrates.

Often crystallizations produce a solvate of a compound of the embodiments disclosed herein. As used herein, the term “solvate” refers to an aggregate that comprises one or more molecules of a compound of the embodiments disclosed herein with one or more molecules of solvent. The solvent may be water, in which case the solvate may be a hydrate. Alternatively, the solvent may be an organic solvent. Thus, the compounds of the embodiments disclosed herein may exist as a hydrate, including a monohydrate, dihydrate, hemihydrate, sesquihydrate, trihydrate, tetrahydrate and the like, as well as the corresponding solvated forms. The compounds of the embodiments disclosed herein may be true solvates, while in other cases, a compound of the embodiments disclosed herein may merely retain adventitious water or be a mixture of water plus some adventitious solvent.

Also, within the scope of the present disclosure are so-called “prodrugs” of the compounds disclosed herein. Thus, certain derivatives of the compounds disclosed herein that may have little or no pharmacological activity themselves can, when administered into or onto the body, be converted into the compounds of the disclosure having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T. Higuchi and W. Stella) and “Bioreversible Carriers in Drug Design,” Pergamon Press, 1987 (ed. E. B. Roche, American Pharmaceutical Association). Prodrugs in accordance with the disclosure can, for example, be produced by replacing appropriate functionalities present in the compounds of the present disclosure with certain moieties known to those skilled in the art as “pro-moieties” as described, for example, in “Design of Prodrugs” by H. Bundgaard (Elsevier, 1985).

A “pharmaceutical composition” refers to a formulation of a compound of the embodiments disclosed herein and a medium generally accepted in the art for the delivery of the biologically active compound to mammals, e.g., humans. Such a medium includes all pharmaceutically acceptable excipients. “Effective amount” or “therapeutically effective amount” refers to an amount of a compound according to the embodiments disclosed herein, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the embodiments disclosed herein which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the embodiments disclosed herein, and the age, body weight, general health, sex and diet of the patient. Such a therapeutically effective amount can be determined routinely by one of ordinary skill in the art having regard to their own knowledge, the state of the art, and this disclosure.

“Effective amount” or “therapeutically effective amount” refers to an amount of a compound according to the embodiments disclosed herein, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the embodiments disclosed herein which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the embodiments disclosed herein, and the age, body weight, general health, sex and diet of the patient. Such a therapeutically effective amount can be determined routinely by one of ordinary skill in the art having regard to their own knowledge, the state of the art, and this disclosure.

The term “treatment” as used herein is intended to mean the administration of a compound or composition according to the present embodiments disclosed hereinto alleviate or eliminate symptoms of the conditions described herein.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

### EXAMPLES

### Formula I Compounds

### Example 1—Synthesis of MB-03

### 8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylic acid

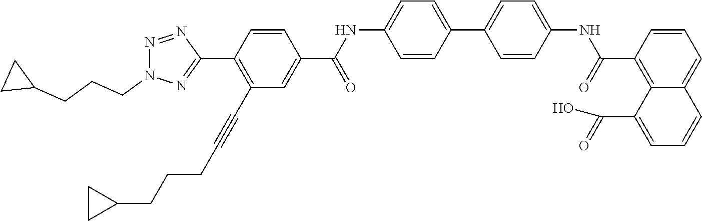 [SMILES: O=C(Nc1ccc(-c2ccc(NC(=O)c3cccc4cccc(C(=O)O)c34)cc2)cc1)c1ccc(-c2nnn(CCCC3CC3)n2)c(C#CCCCC2CC2)c1]

MB-03-R′-Left Synthesis:

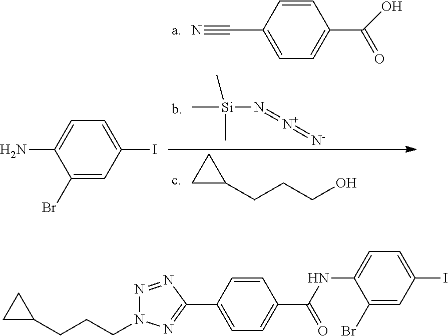

### MB-03-R′-left synth Synthesis of N-(2-bromo-4-iodophenyl)-4-[2-(3-cyclopropylpropyl)-2H-1,2,3,4-tetrazol-5-yl]benzamide

a. 2-Bromo-4-iodo-aniline is combined with 4-cyanobenzoic acid to form N-(2-bromo-4-iodophenyl-4-cyanobenzamide using General Procedure I (see, Example 2)

b. trimethylsilyl azide N-(2-bromo-4-iodophenyl)-4-(2H-1,2,3,4-tetrazol-5-yl)benzamide General Procedure P: Conversion of nitrile to tetrazole with trimethylsilyl azide Specific example: Synthesis of N-(2-bromo-4-iodophenyl)-4-(2H-1,2,3,4-tetrazol-5yl)benzamide To a reaction vial equipped with a magnetic stirrer are added N-(2-bromo-4-iodophenyl-4-cyanobenzamide (0.07 mmol, 1 equiv), trimethylsilyl azide (12 mg, 0.105 mmol, 1.5 equiv) and tetrabutylammonium fluoride (TBAF, 9.2 mg, 0.035 mmol, 0.5 equiv), and a minimum amount of THF to dissolve all components at 85° C. The resulting mixture is heated under stirring at 85° C. for 3 days. The crude reaction mixture is dissolved in ethyl acetate (10 mL) and TBAF is removed by washing the organic phase with 1 M HCI aqueous solution (3×5 mL). The organic layer is dried (Na₂SO₄) and concentrated in vacuo. The residue is optionally purified by silica gel chromatography (CH₂Cl₂/MeOH gradient) to yield the title product.

c. General Procedure Q: Alkylation of tetrazole on 2 position with alcohol via Mitsunobu reaction. Specific example: Synthesis of N-(2-bromo-4-iodophenyl)-4-[2-(3-cyclopropylpropyl)-2H-1,2,3,4-tetrazol-5-yl]benzamide To a stirred mixture of the N-(2-bromo-4-iodophenyl)-4-(2H-1,2,3,4-tetrazol-5yl)benzamide (0.996 mmol) and 3-Cyclopropyl-propan-1-ol (1.0 mmol) in dichloromethane at 5° C. under nitrogen is added in one portion triphenylphosphine (262 mg, 0.999 mmol) followed by the dropwise addition of neat diethyl azodicarboxylate (0.16 ml, 1.0 mmol) over 10 minutes. The resulting mixture is stirred briefly, then allowed to warm to room temperature. After 22 h, the solvent is rotary evaporated, and the residue is purified by silica gel chromatography eluting with a hexanes/ethyl acetate or methylene chloride/methanol gradient to yield the title product.

MB-03-R(Right) Synthesis:

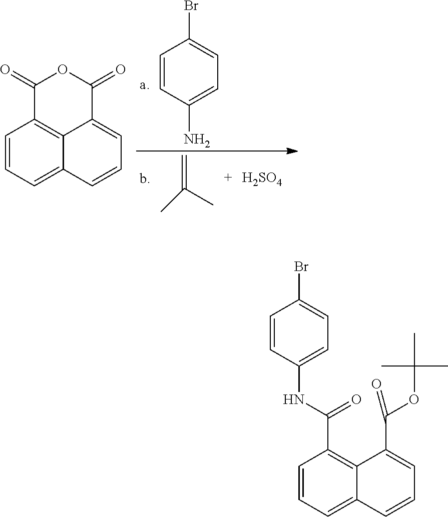

a. Synthesis of 8-[(4-bromophenyl)carbamoyl]naphthalene-1-carboxylic acid. To a 100 mL round-bottomed flask equipped with a magnetic stir bar is added 4-bromoaniline (31.3 mmol), dichoromethane (1000 mL), DMF (1000 mL) and 1,8-naphthalic anhydride (31.3 mmol). The stirring solution is allowed to stir for 24 hours at room temperature. Volatiles are evaporated under reduced pressure, and the residue is washed with dry ethyl acetate and methylene chloride and warmed under high vacuum to 40° C. for 18 hours to remove traces of water. The resulting crude product is used for the next step without purification.

b. Synthesis of t-butyl carboxylates from carboxylic acids: General Procedure G Specific Example: Synthesis of tert-butyl 8-[(4-bromophenyl)carbamoyl]naphthalene-1-carboxylate

A slurry of 8-[(4-bromophenyl)carbamoyl]naphthalene-1-carboxylic acid (9.2 mmol) in dioxane (9 mL) and concentrated H₂SO₄ (0.5 mL) is cooled to 0° C., and then bubbled through with isobutene for 2 h. The reaction is allowed to gradually warm up to room temperature overnight. Solid NaHCO₃ (4 g) is carefully added to the reaction and the mixture is stirred for 1 h. The mixture is concentrated, and then redissolved in water and ethyl acetate. The layers are separated. The aqueous phase is washed with ethyl acetate. The combined organics are washed with sat aqueous NaHCO₃ and brine, then dried over Na₂SO₄, filtered and concentrated in vacuo. The product is purified by silica gel chromatography using hexane/ethyl acetate or methylene chloride/methanol.

Alternately and optionally, the synthesis of t-butyl carboxylates from carboxylic acids may be carried out without acid using carbonyldiimidazole and t-butanol. See procedure H in example 11.

### Final Assembly Steps 3 and 4:

### In Synthesis of MB-03 (8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylic acid)

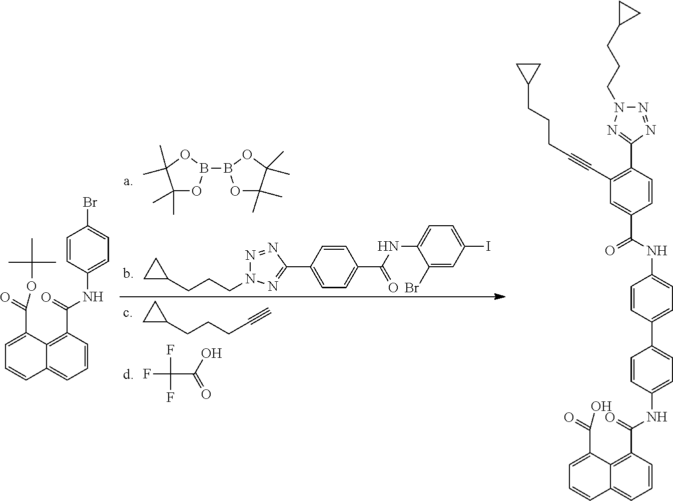

a. Tert-butyl 8-[(4-bromophenyl)carbamoyl]naphthalene-1-carboxylate and bis(pinacolato)diboron are combined using the procedure in step a of General Procedure Z (see Example 2), with the following change: The solvent (isopropanol or n-butanol) is removed in vacuo, and the resulting crude tert-butyl 8-[(4-(pinacolatoborophenyecarbamoyl]naphthalene-1-carboxylate is purified using silica gel chromatography using a methanol/DCM or ethyl acetate/hexane gradient.

b. Synthesis of tert-butyl 8-[(4′-{3-bromo-4-[2-(3-cyclopropylpropyl)-2H-1,2,3,4-tetrazol-5-yl]benzamido}-[1,1′-biphenyl]-4-yl)carbamoyl]naphthalene-1-carboxylate 3-Bromo-4-[2-(3-cyclopropylpropyl)-2H-1,2,3,4-tetrazol-5-yl]-N-(4-iodophenyl) benzamide and tert-butyl 8-[(4-(pinacolatoborophenyl) carbamoyl]naphthalene-1-carboxylate are combined using General Procedure W (see Example 10) to form the title product.

c. General Procedure F: Sonogashira Coupling Of Alkyne and Aryl Halide to form Aryl alkyne. Specific Example: Synthesis of t-butyl 8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylate:

Na₂PdCl₄ (0.05 mmol), t-butyl-dicyclohexylphosphine hydrochloride (0.05 mmol), and CuI (0.05 mmol) are weighed in an oven-dried two-necked Schlenk-flask equipped with a reflux condenser. Diisopropylamine (50 mL) is transferred to the flask via cannula. Tert-butyl 8-[(4′-{3-bromo-4-[2-(3-cyclopropylpropyl)-2H-1,2,3,4-tetrazol-5-yl]benzamido}-[1,1′-biphenyl]-4-yl)carbamoyl]naphthalene-1-carboxylate (10 mmol) is transferred to the flask with a syringe and the mixture carefully degassed via “freeze and thaw” technique. After being warmed to rt, the mixture is warmed and stirred at 80° C. for 10 min. If the material does not dissolve, additional diisopropylamine is added and the mixture stirred for an additional 10 minutes. 5-cyclopropylpent-1-yne (10.5 mmol) is added via syringe. After onset of the reaction is observed (precipitation of H₂N-i-Pr₂Br and a darkening of the reaction mixture), stirring is continued for 4 to 10 hours, following the reaction by TLC. The reaction is stopped either when there is no further decrease in aryl bromide, or when all of the aryl bromide has been consumed. After the mixture is cooled to room temperature, the precipitate is separated via suction filtration (glass frit G4) and washed twice with HNi—Pr₂. The volatiles are evaporated in vacuo. The residue is purified by column chromatography using cyclohexane/ethyl acetate or methylene chloride/methanol mixtures as the eluent to yield t-butyl 8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylate.

d. t-Butyl 8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylate is deprotected with TFA to form the final product MB-03=8-({4′-[3-(5-cyclopropylpent-1-yn-1-yl)-4-[2-(3-cyclopropylpropyl)-2H-1,2,3,4-tetrazol-5-yl]benzamido]-[1,1′-biphenyl]-4-yl}carbamoyl)naphthalene-1-carboxylic acid using General Procedure X (see Example 12).

### Example-2 Synthesis MB-04

### 5-({4′-[3-(1-butyl-1H-1,2,3-triazol-4-yl)-4-(2-butyl-2H-1,2,3,4-tetrazol-5-yl)benzamido]-[1,1′-biphenyl]-4-yl}carbamoyl)naphthalene-1-carboxylic acid

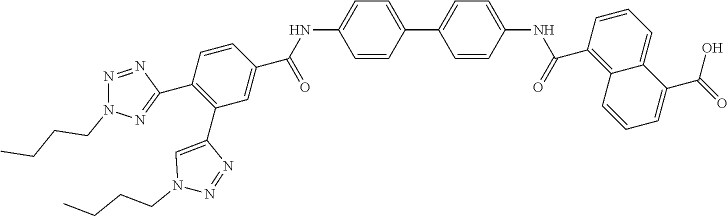 [SMILES: CCCCn1cc(-c2cc(C(=O)Nc3ccc(-c4ccc(NC(=O)c5cccc6c(C(=O)O)cccc56)cc4)cc3)ccc2-c2nnn(CCCC)n2)nn1]

### MB-04-R′-left synthesis: N-(4-bromophenyl)-3,4-bis(2-butyl-2H-1,2,3,4-tetrazol-5-yl)benzamide

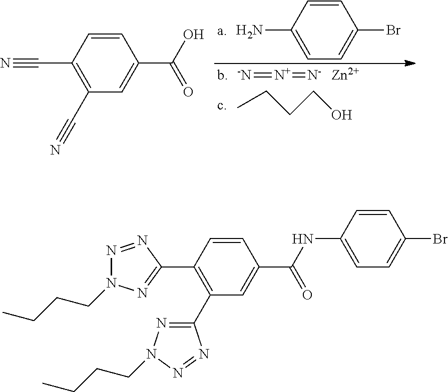

a. 3,4-Dicyano-benzoic acid is combined with 4-bromoaniline to form N-(4-bromophenyl)-3,4-dicyanobenzamide using General Procedure I (see Example 2). b. General Procedure R: Conversion of nitrile to tetrazole with zinc azide. Specific Example: Synthesis of N-(4-bromophenyl)-3,4-bis(2H-1,2,3,4-tetrazol-5-yl)benzamide To a 250 mL round-bottomed flask is added N-(4-bromophenyl)-3,4-dicyanobenzamide (20 mmol), sodium azide (1.43 g, 22 mmol), zinc bromide (4.50 g, 20 mmol), and 40 mL of water. The reaction mixture is refluxed for 24 h with vigorous stirring and followed by TLC. (Optionally, the reaction is run in a pressure tube submerged up to the neck in an oil bath at 140° C.-170° C. for 24-48 hours.) HCl (3 N, 30 mL) and ethyl acetate (100 mL) are added, and vigorous stirring is continued until no solid is present and the aqueous layer had a pH of 1. If necessary, additional ethyl acetate is added. The organic layer is isolated and the aqueous layer extracted with 2 100 mL of ethyl acetate. The combined organic layers are evaporated, 200 mL of 0.25 N NaOH is added, and the mixture is stirred for 30 min, until the original precipitate is dissolved and a suspension of zinc hydroxide is formed. The suspension is filtered, and the solid washed with 20 mL of 1 N NaOH. To the filtrate is added 40 mL of 3 N HCl with vigorous stirring causing the tetrazole to precipitate. The product is filtered and washed twice with 20 mL of 3 N HCl and dried in a drying oven to furnish the title product. c. N-(4-bromophenyl)-3,4-bis(2H-1,2,3,4-tetrazol-5-yl)benzamide is combined with butan-1-ol to form N-(4-bromophenyl)-3,4-bis(2-butyl-2H-1,2,3,4-tetrazol-5-yl)benzamide using General Procedure Q (see Example 1).

### Synthesis MB-04-(right)=tert-butyl 5-[(4-bromophenyl)carbamoyl]naphthalene-1-carboxylate

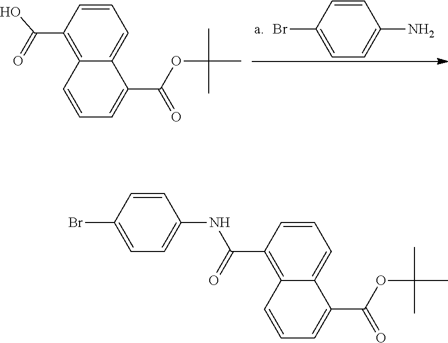 [SMILES: CC(C)(C)OC(=O)c1cccc2c(C(=O)O)cccc12 ; Nc1ccc(Br)cc1 ; CC(C)(C)OC(=O)c1cccc2c(C(=O)Nc3ccc(Br)cc3)cccc12]

General Procedure I: Synthesis of N-aryl amides from Arylamines and carboxylic acids: Specific Example: synthesis of tert-butyl 5-[(4-bromophenyl)carbamoyl]naphthalene-1-carboxylate tert-butyl 5-[(4-bromophenyl)carbamoyl]naphthalene-1-carboxylate To a solution of 5-[(tert-butoxy)carbonyl]naphthalene-1-carboxylic acid (1.59 mmol) in CH₂Cl₂ (8 mL) at 0° C. are added 4-bromoaniline (1.43 mmol) and triethylamine (0.78 ml, 5.38 mmol). After stirring the reaction mixture for 10 minutes, 1-propanephosphonic acid cyclic anhydride (50 percent solution in ethyl acetate, 2.03 ml, 3.18 mmol) is added via syringe and stirred at room temperature. After 16 hours, the reaction is diluted with water and extracted with ethyl acetate. The organic layers are combined and dried over magnesium sulfate, filtered, and concentrated under reduced pressure. The resulting residue is purified by flash chromatography (silica gel) with EtOAc/hexane and stripped of solvent in vacuo.

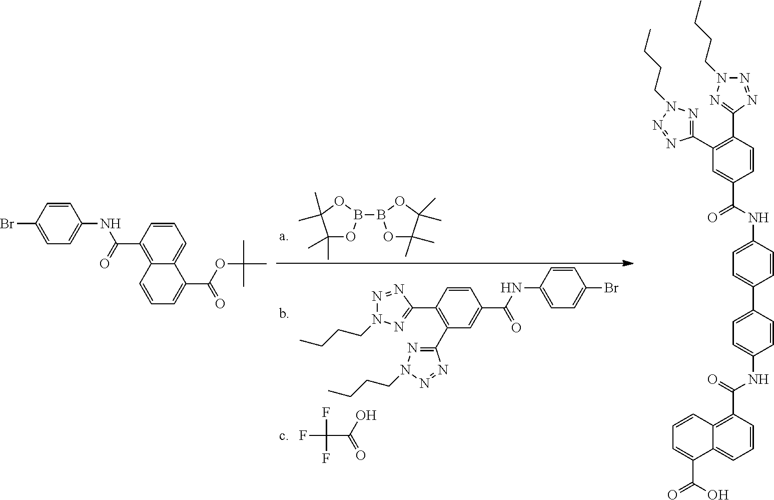

Steps a, b, and c. General Procedure Z: ArX-right+Ar′X-left coupling to form the Ar′-Ar-t-butyl ester in a 1 pot, 2 step procedure. Specific example:

### a. MB-04 final assembly step 3 Synthesis of t-butyl-5-({4′-[3-(1-butyl-1H-1,2,3-triazol-4-yl)-4-(2-butyl-2H-1,2,3,4-tetrazol-5-yl)benzamido]-[1,1′-biphenyl]-4-yl}carbamoyl)naphthalene-1-carboxylate.

a. MB-04-R(right)=tert-butyl 5-[(4-bromophenyl)carbamoyl]naphthalene-1-carboxylate (10 mmol, 1 equiv), Bis(pinacolato)-diboron (11 mmol, 1.1 equiv), KOAc (22 mmol, 2.2 equiv), 28 mL anhydrous iPrOH (0.75 M molar concentration with respect to reagents), 2 mol % SiliaCat DPP-Pd (0.25 mmol/g palladium loading), at 82° C. The reaction is followed by TLC until complete. The reaction mixture is used “as is” for step b, with no purification. [Alternately, the borylation reaction can be is carried out at 98° C. in 21 mL anhydrous 2-BuOH using 10 mmoles Bis(pinacolato)diboron (B2Pin2), and the solvent is removed in vacuo and replaced with 28 mL anhydrous iPrOH prior to step b.] SiliaCat DPP-Pd is available from SilaCycle, Quebec City, Canada.

b. The reaction mixture from step a (isopropanol solvent) is treated with MB-04-R′(left)=N-(4-bromophenyl)-3 ,4-bis (2-butyl-2H-1,2,3,4-tetrazol-5-yl)benzamide (12 mmol), K₂CO₃ (23 mmol, 2.3 equiv relative to substrate 1), and 8 mL distillated H₂O in a flask with a reflux condenser. (The reaction solvent becomes iPrOH/H₂O, 3.5:1, v/v). The reaction is heated at 82° C. with stirring under nitrogen or argon and followed by TLC until complete. The product is purified with silica gel chromatography using a methanol/DCM or ethyl acetate/hexane gradient, and stripped of solvent in vacuo to yield t-Butyl-5-({4′-[3-(1-butyl-1H-1,2,3-triazol-4-yl)-4-(2-butyl-2H-1,2,3,4-tetrazol-5-yl)benzamido]-[1,1′-biphenyl]-4-yl}carbamoyl)naphthalene-1-carboxylate.

c. t-Butyl-5-({4′-[3-(1-butyl-1H-1,2,3-triazol-4-yl)-4-(2-butyl-2H-1,2,3,4-tetrazol-5-yl)benzamido]-[1,1′-biphenyl]-4-yl}carbamoyl)naphthalene-1-carboxylate is deprotected with trifluoroacetic acid to form the final product MB-04=5-({4′-[3-(1-butyl-1H-1,2,3-triazol-4-yl)-4-(2-butyl-2H-1,2,3,4-tetrazol-5-yl)benzamido]-[1,1′-biphenyl]-4-yl}carbamoyl)naphthalene-1-carboxylic acid using General Procedure W (see Example 10).

### Example 3—Synthesis of MB-06

### 8-[(4′-{[3-(2-hexyl-2H-1,2,3,4-tetrazol-5-yl)-4-(methoxycarbonyl)phenyl]carbamoyl}-[1,1′-biphenyl]-4-yl)carbamoyl]naphthalene-1-carboxylic acid

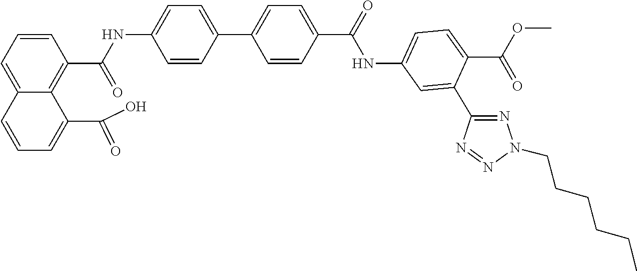 [SMILES: CCCCCCn1nnc(-c2cc(NC(=O)c3ccc(-c4ccc(NC(=O)c5cccc6cccc(C(=O)O)c56)cc4)cc3)ccc2C(=O)OC)n1]

### MB-06-R′-left synthesis: Synthesis of methyl 4-(4-bromobenzamido)-2-(2-hexyl-2H-1,2,3,4-tetrazol-5-yl)benzoate

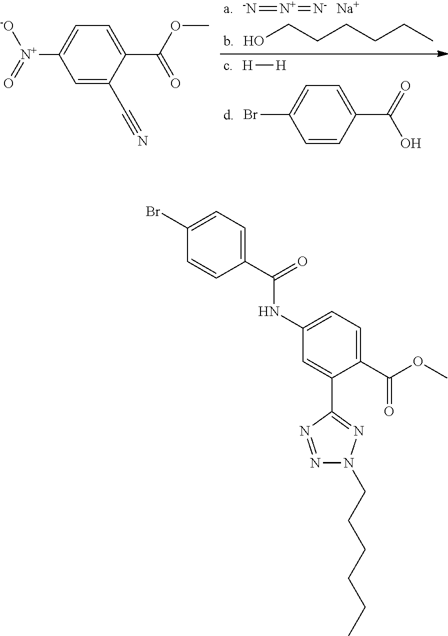

a. General Procedure S: Conversion of nitrile to tetrazole with sodium azide Specific example: Synthesis of methyl 4-nitro-2-(2H-1,2,3,4-tetrazol-5-yl)benzoate. A mixture of methyl 2-cyano-4-nitrobenzoate (26.11 mmol, 1.0 eq), NaN₃ (5.1 g, 78.33 mmol, 3.0 eq) and triethylamine hydrochloride (10.8 g, 78.33 mmol, 3.0 eq) in toluene (100 mL) is heated overnight at 100° C. oil bath. The completion of the reaction is monitored by analytical HPLC or TLC. When complete, the reaction mixture is cooled to RT and concentrated to provide the crude which is purified by column chromatography on silica gel using hexane/ethyl acetate or methylene chloride/methanol gradient to obtain the title product.

Alternately and optionally, General Procedure P (trimethylsilyl azide, see Example 1) or R (zinc azide, see Example 2) can be used to convert the nitrile to the tetrazole ring using.

b. Methyl 4-nitro-2-(2H-1,2,3,4-tetrazol-5-yl)benzoate is combined with hexan-1-ol to form methyl 2-(2-hexyl-2H-1,2,3,4-tetrazol-5-yl)-4-nitrobenzoate using General Procedure I (see Example 2).

c. General Procedure T: Reduction of nitroarene to aminoarene with hydrogen: Specific example: synthesis of methyl 4-amino-2-(2-hexyl-2H-1,2,3,4-tetrazol-5-yl)benzoate To a solution of methyl 2-(2-hexyl-2H-1,2,3,4-tetrazol-5-yl)-4-nitrobenzoate (42.5 mmol, 1.O eq) in MeOH (100 mL), 10 percent Pd/C (2.0 g) is added. Hydrogen is purged through the reaction mixture for 4 h. After completion of the reaction, mixture is filtered through Celite-bed and washed with MeOH. The filtrate is concentrated under reduced pressure, and optionally further purified on silica using a DCM/MeOH gradient to yield the title product.

d. Methyl 4-amino-2-(2-hexyl-2H-1,2,3,4-tetrazol-5-yl)benzoate is combined with 4-bromobenzoic acid t form methyl 4-(4-bromobenzamido)-2-(2-hexyl-2H-1,2,3,4-tetrazol-5-yl)benzoate using General Procedure Q (see Example 1). Final Assembly: MB-06 is synthesized from MB-06-R′(left) and MB-03-R(right) using General Procedures Z and W as in Example 2.

### Example 4 Synthesis of MB-07: 5-[(6′-{4-[(2-cyclohexylethyl)(methoxy)carbamoyl]benzamido}-[3,3′-bipyridazine]-6-yl)carbamoyl]naphthalene-1-carboxylic acid

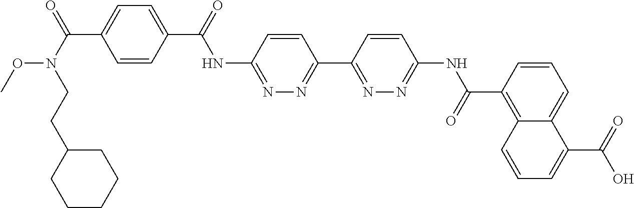 [SMILES: CON(CCC1CCCCC1)C(=O)c1ccc(C(=O)Nc2ccc(-c3ccc(NC(=O)c4cccc5c(C(=O)O)cccc45)nn3)nn2)cc1]

Synthesis scheme MB-07. This molecule does not have an R(right) or an R′(left) because the aryl-aryl coupling step to form the bis([3,3′-bipyridazine]-6,6′-diamine) is carried out prior to attachment of the naphthalenecarboxylate and benzamide units.

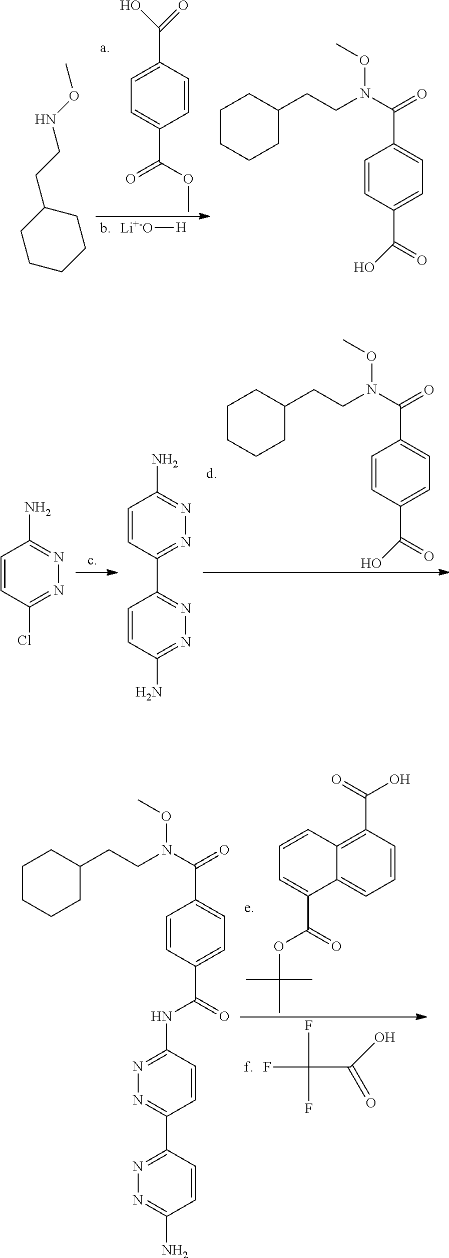

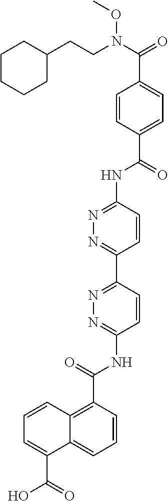 [SMILES: CON(CCC1CCCCC1)C(=O)c1ccc(C(=O)Nc2ccc(-c3ccc(NC(=O)c4cccc5c(C(=O)O)cccc45)nn3)nn2)cc1]

a. Methyl 4-(carboxy)benzoate is combined with (2-cyclohexylethyl)(methoxy)amine to form methyl 4-[(2-cyclohexylethyl)(methoxy)carbamoyl]benzoate using General Procedure Y (see Example 16).

b. Methyl 4-[(2-cyclohexylethyl)(methoxy)carbamoyl]benzoate is hydrolyzed to form 4-[(2-cyclohexylethyl)(methoxy)carbamoyl]benzoic acid using General Procedure O (see Example 24).

c. Synthesis of bis([3,3′-bipyridazine]-6,6′-diamine) A 250 mL flask is charged with 5 grams of 6-Chloropyridazin-3-amine, 5 g of Pd/CaCO3, and 100 mL of 5% methanolic KOH. (Alternately, 5% ethanolic NaOH can be used). To this mixture, 4 mL of 80% hydrazine hydrate solution is added dropwise under vigorous stirring at room temperature. The stirring is continued for 6 hours at room temperature, and the catalyst is removed by filtration. After stripping the solvent in vacuo, the crude product is purified by crystallization from methanol. Alternately, the crude product can be purified by crystallization of the oxalate salt. Alternately, the crude product can be converted to a bis-BOC derivative with BOC anhydride, purified using silica gel, followed by treatment with TFA to cleave the BOC groups and yield the TFA salt.

d. Bis([3,3′-bipyridazine]-6,6′-diamine) was combined with 1 equivalent of 4-[(2-cyclohexylethyl)(methoxy)carbamoyl]benzoic acid to form N4-{6′-amino-[3,3′-bipyridazine]-6-yl}-N1-(2-cyclohexylethyl)-N1-methoxybenzene-1,4-dicarboxamide using General Procedure I (see Example 2).

e. N4-{6′-amino-[3,3′-bipyridazine]-6-yl}-N1-(2-cyclohexylethyl)-N1-methoxybenzene-1,4-dicarboxamide is combined with 5-[(tert-butoxy)carbonyl]naphthalene-1-carboxylic acid to form t-butyl 5-[(6′-{4-[(2-cyclohexylethyl)(methoxy)carbamoyl]benzamido}-[3,3′-bipyridazine]-6-yl)carbamoyl]naphthalene-1-carboxylate using General Procedure I.

f. t-butyl5-[(6′-{4-[(2cyclohexylethyl)(methoxy)carbamoyl]benzamido}-[3,3′-bipyridazine]-6-yl)carbamoyl]naphthalene-1-carboxylate is treated with trifluoroacetic acid to form 5-[(6′-{4-[(2-cyclohexylethyl)(methoxy)carbamoyl]benzamido}-[3,3′-bipyridazine]-6-yl)carbamoyl]naphthalene-1-carboxylic acid using General Procedure X (see Example 12).

### Example-5 Synthesis of MB-08: 4-[3′,5′-difluoro-4′-(5-{4-[methoxy(methyl)carbamoyl]benzamido}-1,3,4-thiadiazol-2-yl-[1,1′-biphenyl]-4-amido]naphthalene-1-carboxylic acid

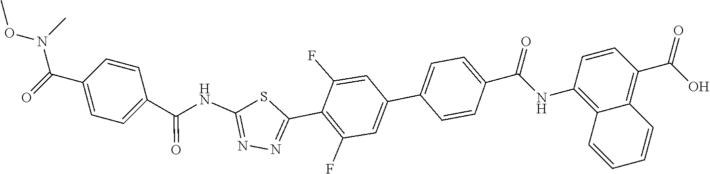 [SMILES: CON(C)C(=O)c1ccc(C(=O)Nc2nnc(-c3c(F)cc(-c4ccc(C(=O)Nc5ccc(C(=O)O)c6ccccc56)cc4)cc3F)s2)cc1]

### MB-08-R′-left synthesis: Synthesis of N-4-[5-(4-bromo-2,6-difluorophenyl)-1,3,4-thiadiazol-2-yl]-N1-methoxy-N1-methylbenzene-1,4-dicarboxamide

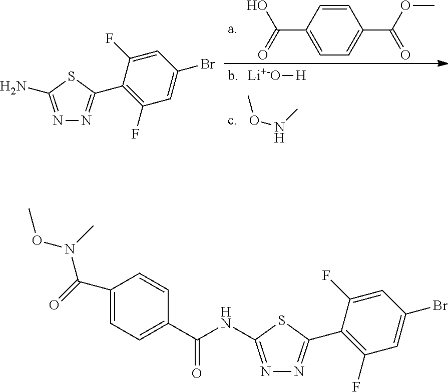

a. 4-Methoxycarbonylbenzoic acid and 5-(4-bromo-2,6-difluorophenyl)-1,3,4-thiadiazol-2-amine are combined to make methyl 4-{[5-(4-bromo-2,6-difluorophenyl)-1,3,4-thiadiazol-2-yl]carbamoyl}benzoate using General Procedure I (see Example 2).

b. Methyl 4-{[5-(4-bromo-2,6-difluorophenyl)-1,3,4-thiadiazol-2-yl]carbamoyl}benzoate was hydrolyzed with lithium hydroxide to form 4-{[5-(4-bromo-2,6-difluorophenyl)-1,3,4-thiadiazol-2-yl]carbamoyl}benzoic acid using General Procedure O (see Example 24).

c. 4-{[5-(4-bromo-2,6-difluorophenyl)-1,3,4-thiadiazol-2-yl]carbamoyl}benzoic acid and methoxy(methyl)amine are combined to make N-4-[5-(4-bromo-2,6-difluorophenyl)-1,3,4-thiadiazol-2-yl]-N1-methoxy-N1-methylbenzene-1,4-dicarboxamide using General Procedure Y (see Example 16).

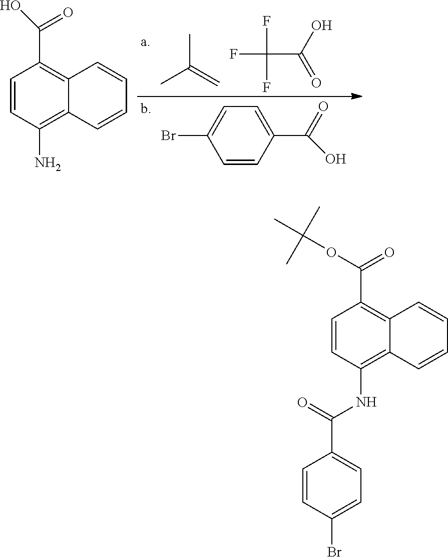

MB-08R-(right) synthesis:

a. 4-amino-1-napthoic acid is combined with isobutene in the presence of trifluoroacetic acid to yield t-butyl-4-amino-1-napthoate using General Procedure G (see Example 1).

b. 4-bromobenzoic acid is combined with t-butyl-4-amino-1-napthoate to yield MB-08R(right)=(4-bromobenzamido)naphthalene-1-carboxylate using General Procedure I (see Example 2).

c. Final Assembly: MB-08 is synthesized from MB-08-R′(left) and MB-08-R(right) using General Procedures Z and W as in Example 2.

### Example 6—Synthesis of MB-10R: 5-[(6′-{4-[4-(2-ethoxy-2-oxoethyl)-1H-1,2,3-triazol-1-yl]benzamido}-[3,3′-bipyridine]-6-yl)carbamoyl]naphthalene-1-carboxylic acid

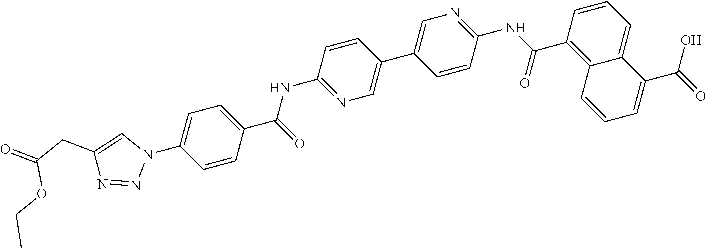 [SMILES: CCOC(=O)Cc1cn(-c2ccc(C(=O)Nc3ccc(-c4ccc(NC(=O)c5cccc6c(C(=O)O)cccc56)nc4)cn3)cc2)nn1]

### MB-10-R′-left synth: Synthesis of ethyl 2-(1-{4-[(5-bromopyridin-2-yl)carbamoyl]phenyl}-1H-1,2,3-triazol-4-yl)acetate

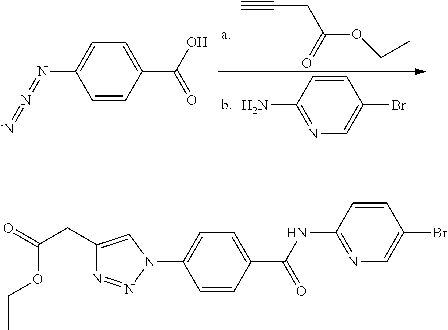

a. General Procedure U: 1,2,3-triazole from Arylazide and alkyne Specific example: Synthesis of 4-[4-(2-ethoxy-2-oxoethyl)-1H-1,2,3-triazol-1-yl]benzoic acid To 0.05 molar aqueous solution of CuSO₄ (1.4 ml), 2-{4-[(dimethylamino)methyl]-1,2,3-triazol-1-yl}cyclohexan-1-ol (AMTC) (32 mg) is added, dissolved in water (3.3 ml). Next, 4-Azidobenzoic acid (1.5 mmoles) and ethyl but-3-ynoate (1.5 mmoles) are added, followed by ethanol (2.4 ml). The reaction is initiated by slowly adding sodium ascorbate (14 mg), dissolved in water (0.1 ml). The reaction mixture is stirred vigorously for 1.5 h at room temperature. After that time, ethanol is evaporated under reduced pressure and the residue is diluted by adding water (20 ml), and extracted with dichloromethane or ethyl acetate (3×15 ml). The product is optionally purified by silica chromatography using a DCM/MeOH gradient.

b. 4-[4-(2-Ethoxy-2-oxoethyl)-1H-1,2,3-triazol-1-yl]benzoic acid is combined with 2-amino-5-bromopyridine to form ethyl 2-(1-{4-[(5-bromopyridin-2-yl)carbamoyl]phenyl}-1H-1,2,3-triazol-4-yl) acetate using General Procedure I (see Example 2).

### MB-10-R(right) synthesis: Synthesis of tert-butyl 5-[(5-bromopyridin-2-yl)carbamoyl]naphthalene-1-carboxylate

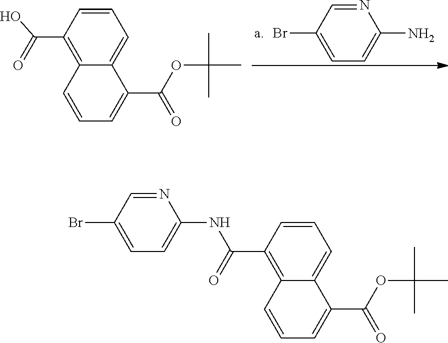 [SMILES: CC(C)(C)OC(=O)c1cccc2c(C(=O)O)cccc12 ; Nc1ccc(Br)cn1 ; CC(C)(C)OC(=O)c1cccc2c(C(=O)Nc3ccc(Br)cn3)cccc12]

5-[(tert-butoxy)carbonyl]naphthalene-1-carboxylic acid and 5-bromopyridin-2-amine are converted to tert-butyl 5-[(5-bromopyridin-2-yl)carbamoyl]naphthalene-1-carboxylate using General Procedure I (see Example 2).
Final Assembly: MB-10 is synthesized from MB-10-R′(left) and MB-10-R(right) using General Procedures Z and W as in Example 2.

### Example-7 Synthesis of MB-11

### 2-{2-[4′-({5-[4-(methoxycarbonyl)phenyl]-1,3,4-thiadiazol-2-yl}carbamoyl)-[1,1′-biphenyl]-4-amido]-6-(propan-2-yl)phenyl}acetic acid

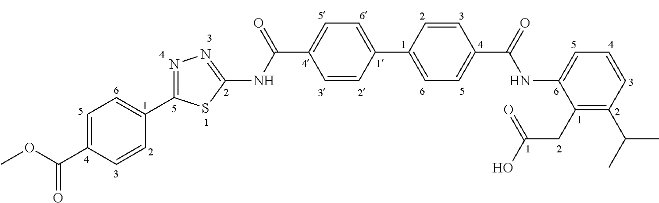 [SMILES: COC(=O)c1ccc(-c2nnc(NC(=O)c3ccc(-c4ccc(C(=O)Nc5cccc(C(C)C)c5CC(=O)O)cc4)cc3)s2)cc1]

### MB-11-left-synthesis: methyl 4-[5-(4-bromobenzamido)-1,3,4-thiadiazol-2-yl]benzoate

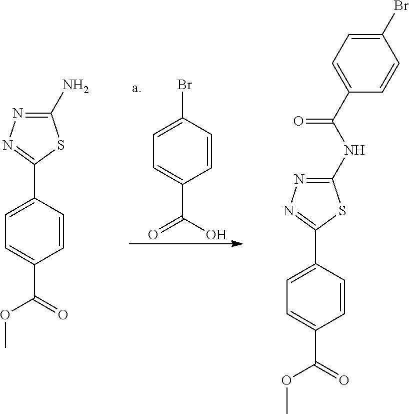 [SMILES: COC(=O)c1ccc(-c2nnc(N)s2)cc1 ; O=C(O)c1ccc(Br)cc1 ; COC(=O)c1ccc(-c2nnc(NC(=O)c3ccc(Br)cc3)s2)cc1]

a. Methyl 4-(5-amino-1,3,4-thiadiazol-2-yl)benzoate is combined with 4-bromobenzoic acid to form methyl 4-[5-(4-bromobenzamido)-1,3,4-thiadiazol-2-yl]benzoate using General Procedure I (see Example 2).

### MB-11-R′(right) synthesis: 1-(4-bromobenzoyl)-4-(propan-2-yl)-2,3-dihydro-1H-indol-2-one

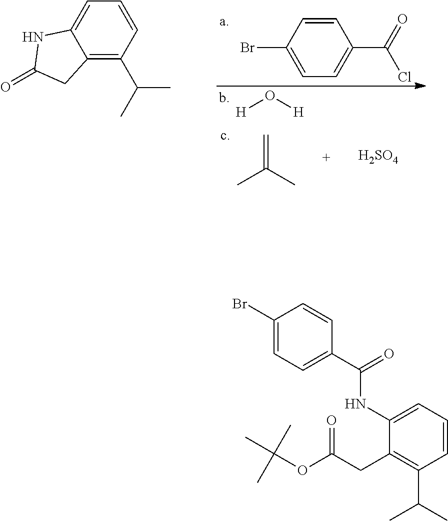

a. A solution of 0.1 mole of 4-(Propan-2-yl)-2,3-dihydro-1H-indol-2-one in 400 ml of toluene is treated with 0.1 mole of 4-bromobenzoyl chloride. The mixture is heated at 70° C. with stirring and a solution of 10.2 g (0.1 mole) of triethylamine in 100 ml of toluene is added dropwise. The mixture is stirred with heating for an additional 16 hours, filtered while warm through filter aid to remove triethylammonium hydrochloride, and concentrated at reduced pressure. Chromatography over silica gel using methanol/dichloromethane yields, after removal of solvent in vacuo, 1-(4-bromobenzoyl)-4-(propan-2-yl)-2,3-dihydro-1H-indol-2-one.

b. Synthesis of 2-[2-(4-bromobenzamido)-6-(propan-2-yl)phenyl]acetic acid. 1-(4-Bromobenzoyl)-4-(propan-2-yl)-2,3-dihydro-1H-indol-2-one is heated with a 9:1 mixture of dioxane and water at 80° C. for 0.5 h until the reaction is complete by TLC. The reaction mixture is stripped of solvent. The crude product is dissolved in ethyl acetate, methylene chloride, chloroform, or a mixture, dried over anhydrous Na₂SO₄, filtered, and stripped of solvent to form 2-[2-(4-bromobenzamido)-6-(propan-2-yl)phenyl]acetic acid. The crude intermediate is used for the final step without further purification.

c. Synthesis of t-butyl carboxylates from carboxylic acids with carbonyldiimidazole: General Procedure H Specific Example: synthesis of t-butyl 2-[2-(4-bromobenzamido)-6-(propan-2-yl)phenyl]acetate 16 mmol of 2-[2-(4-bromobenzamido)-6-(propan-2-yl)phenyl]acetic acid is suspended in 44 mL of dry tert-butyl alcohol and 3.3 mL of dry triethylamine To this is added 3.1 g (19 mmol) of carbonyldiimidazole. The reaction mixture is stirred at room temperature for 12 h and followed by TLC. If the reaction is incomplete, another 5 mmol of carbonyldiimidazole is added and the reaction is warmed to 45° C. Once the reaction is complete by TLC, it is diluted with EtOAc, filtered through diatomaceous earth and concentrated in vacuo. The product is purified on silica gel, eluting with a EtOAc-hexanes gradient, and stripped of solvent to provide t-butyl 2-[2-(4-bromobenzamido)-6-(propan-2-yl)phenyl]acetate (MB-10-R(right)).

Final Assembly: MB-11 is synthesized from MB-11-R′(left) and MB-11-R(right) using General Procedures Z and W as in Example 2.

### Example 8: Synthesis of MB-13

### 8-({6′-[4-(methoxycarbonyl)benzamido]-[3,3′-bipyridazine]-6-yl}carbamoyl)naphthalene-1-carboxylic acid

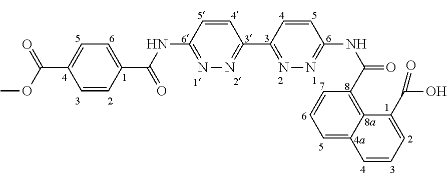 [SMILES: COC(=O)c1ccc(C(=O)Nc2ccc(-c3ccc(NC(=O)c4cccc5cccc(C(=O)O)c45)nn3)nn2)cc1]

This molecule does not have an R(right) or an R′(left) because the aryl-aryl coupling step to form the bis([3,3′-bipyridazine]-6,6′-diamine) is carried out prior to attachment of the naphthalenecarboxylate and benzamide units.

Synthesis Scheme:

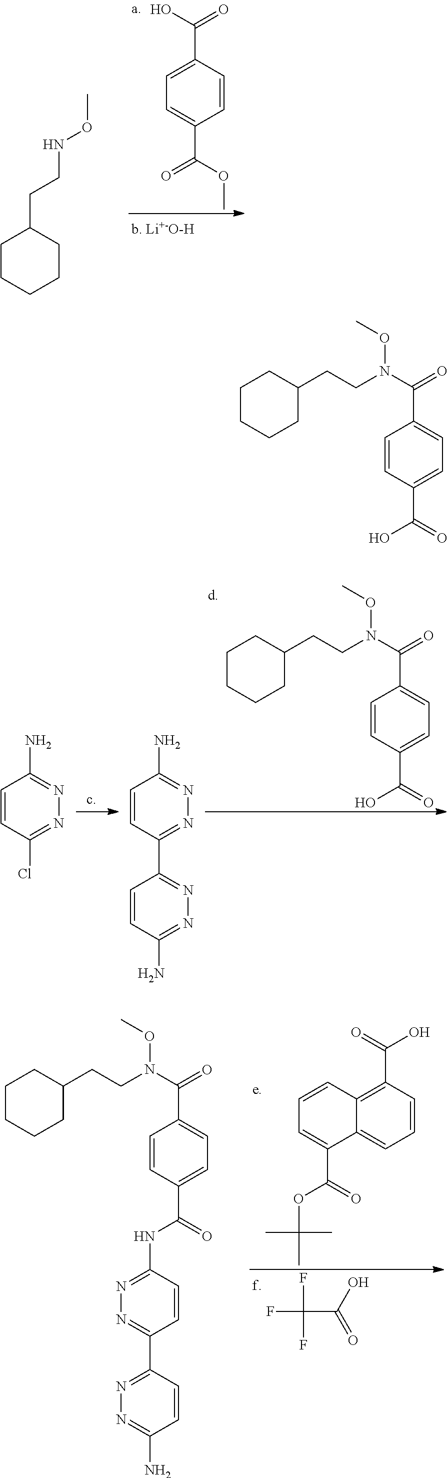

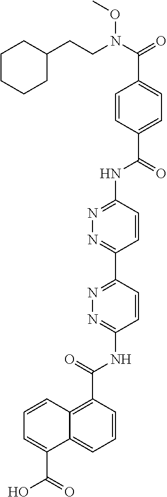 [SMILES: CON(CCC1CCCCC1)C(=O)c1ccc(C(=O)Nc2ccc(-c3ccc(NC(=O)c4cccc5c(C(=O)O)cccc45)nn3)nn2)cc1]

a. For the synthesis of bis([3,3′-bipyridazine]-6,6′-diamine) see Example 4, step c.

b. Bis([3,3′-bipyridazine]-6,6′-diamine) is combined with methyl 4-(carboxy)benzoate to form methyl 4-({6′-amino-[3,3′-bipyridazine]-6-yl}carbamoyl)benzoate using General Procedure I (see Example 2).

c. Synthesis of 8-({6-[4-(methoxycarbonyl)benzamido]-[3,3′-bipyridazine]-6-yl}carbamoyl)naphthalene-1-carboxylic acid To a 3 liter round bottomed flask equipped with a magnetic stir bar is added methyl 4-({6′-amino-[3,3′-bipyridazine]-6-yl}carbamoyl)benzoate (31.3 mmol), dichloromethane (1000 mL), DMF (1000 mL) and 1,8-naphthalic anhydride (31.3 mmol). The solution is allowed to stir for 24 hours at room temperature. Volatiles are evaporated under reduced pressure, and the product is purified by recrystallization from water or methanol and dried in vacuo. Optionally, the crude product is converted to the t-butyl ester using General Procedure G (see Example 1), purified using silica gel chromatography, reconverted to the title product using General Procedure X (see Example 12), and dried in vacuo.

### Example 9—Synthesis of R1Q-01

### 8-(5-{5-[2-(4-methoxy-4-oxobutyl)-1,1,3-trioxo-2,3-dihydro-1 lambda6,2-benzothiazole-6-amido]phenyl}pyridine-2-amido)naphthalene-1-carboxylic acid

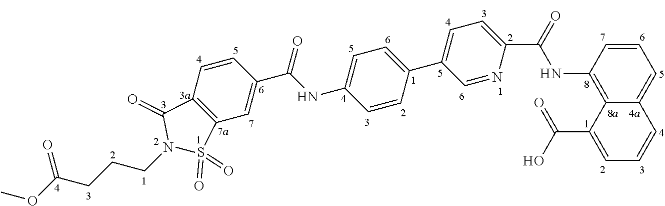 [SMILES: COC(=O)CCCN1C(=O)c2ccc(C(=O)Nc3ccc(-c4ccc(C(=O)Nc5cccc6cccc(C(=O)O)c56)nc4)cc3)cc2S1(=O)=O]

### R1Q-01-R′ left synthesis: ethyl 4-{6-[(4-bromophenyl)carbamoyl]-1, 1,3-trioxo-2,3-dihydro-2-benzothiazol-2-yl}butanoate

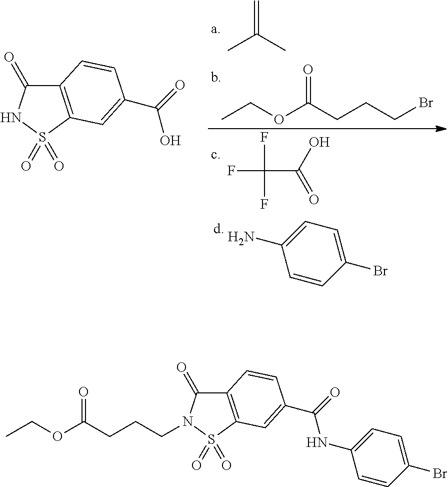

a. 1,1,3-Trioxo-2,3-dihydro-2-benzothiazole-6-carboxylic acid is combined with isobutene to form t-butyl 1,1,3-trioxo-2,3-dihydro-2-benzothiazole-6-carboxylate using General Procedure G.

b. tert-butyl 2-(4-ethoxy-4-oxobutyl)-1,1,3-trioxo-2,3-dihydro-2-benzothiazole-6-carboxylate t-butyl 1,1,3-trioxo-2,3-dihydro-2-benzothiazole-6-carboxylate (4 mmol) is dissolved in 5 ml DMF. Sodium bicarbonate (0.34 g, 4 mmol) and ethyl (4-bromobutyrate) (4 mmol) are added to the solution. The mixture is poured into 50 ml of water after stirred for 4 h at 80° C. The precipitated product is filtrate and optionally purified by recrystallization or using silica chromatography with a DCM/MeOH gradient.

c. tert-Butyl 2-(4-ethoxy-4-oxobutyl)-1,1,3-trioxo-2,3-dihydro-2-benzothiazole-6-carboxylate is treated with TFA to form 2-(4-ethoxy-4-oxobutyl)-1,1,3-trioxo-2,3-dihydro-2-benzothiazole-6-carboxylic acid using General Procedure X (see Example 12).

d. 2-(4-Ethoxy-4-oxobutyl)-1, 1,3-trioxo-2,3-dihydro-2-benzothiazole-6-carboxylic acid is combined with 4-bromoaniline to form ethyl 4-{6-[(4-bromophenyl)carbamoyl]-1,1,3-trioxo-2,3-dihydro-2-benzothiazol-2-yl}butanoate using General Procedure I (see Example 2).

R1Q-01-R(right) synthesis:

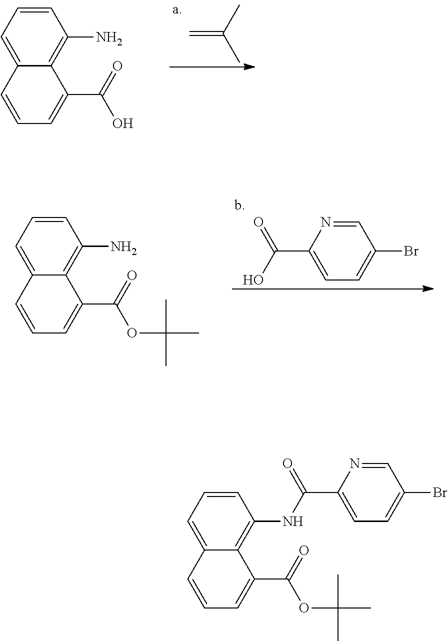

Final Assembly: R1Q-01 is synthesized from R1Q-01-R′(left) and R1Q-01-R(right) using General Procedures Z and W as in Example 2.

### Example 10—Synthesis of R1Q-04

### 8-[5-(4-{N-[2-(2-methoxyethoxy)ethyl]4′-(methoxycarbonyl)-[1,1′-biphenyl]-4-sulfonoimidamido}phenyl)pyridine-2-amido]naphthalene-1-carboxylic acid

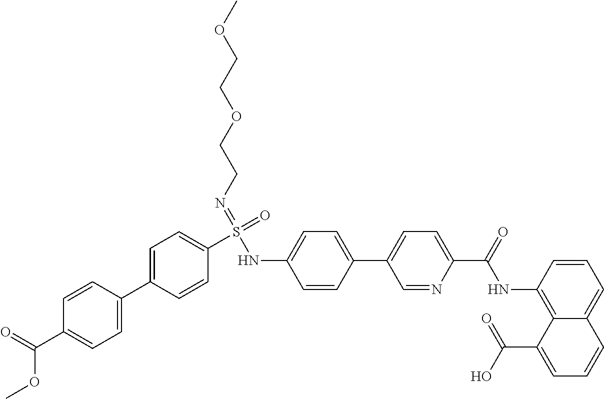 [SMILES: COCCOCCN=S(=O)(Nc1ccc(-c2ccc(C(=O)Nc3cccc4cccc(C(=O)O)c34)nc2)cc1)c1ccc(-c2ccc(C(=O)OC)cc2)cc1]

### Synthesis of R1Q-04-R′(left): methyl 4′-[(4-bromophenyl)[2-(2-methoxyethoxy)ethyl]-S-aminosulfonimidoyl]-[1,1′-biphenyl]-4-carboxylate

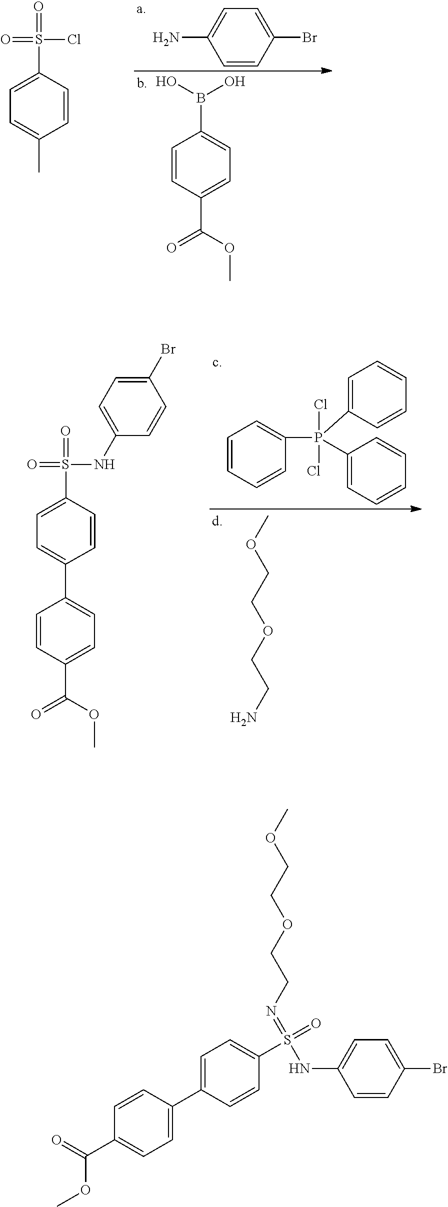

a. Synthesis of N-(4-bromophenyl)-4′-iodo benzenesulfonamide To a solution of 4-bromoaniline (2.00 g, 12.1 mmol) in THF (121 mL) are added 4-Iodobenzene-1-sulfonyl chloride (36.3 mmol) and pyridine (9.76 mL, 121 mmol), and the mixture is stirred room temperature for 1.5 h. The reaction is then quenched with H₂O, and the whole is extracted with EtOAc. The organic layer is washed with brine and H₂O, dried over anhydrous MgSO₄ and evaporated. The residue is purified by silica gel column chromatography (n-hexane/EtOAc gradient) to give Synthesis of N-(4-bromophenyl)-4′-iodo benzenesulfonamide.

b. General Procedure W: Preferential Suzuki coupling at iodoarene in presence of bromoarene. Specific example: Synthesis of methyl 4′-[(4-bromophenyl)sulfamoyl]-[1,1′-biphenyl]-4-carboxylate Into a three neck flask, [4-(methoxycarbonyl)phenyl]boronic acid (17.9 mmol), N-(4-bromophenyl)4′-iodo benzenesulfonamide, (21.5 mmol) and Pd(PPh₃)₄ (0.62 g, 0.54 mmol) are combined under an inert atmosphere. Dimethylformamide (50 milliliter) and an aqueous solution (27 milliliter) of potassium carbonate (7.42 g, 53.7 mmol) are added to the resultant solution and then, it is refluxed under heating for 8 hours. The resultant reaction solution is extracted by toluene, followed by vacuum concentration. The crude product is purified using silica gel column chromatography using a hexane/ethyl acetate or methylene chloride/methanol gradient to obtain the title product.

Steps c, and d. Methyl 4′-[(4-bromophenyl)sulfamoyl]-[1,1′-biphenyl]-4-carboxylate is treated with 1.1 equivalent of dichlorotriphenylphosphorane in the presence of 1.2 equivalents of triethylamine in chloroform at 25° C. The reaction is followed by adding small aliquots to excess octylamine, heating and running TLC until the methyl 4′-[(4-bromophenyl)sulfamoyl]-[1,1′-biphenyl]-4-carboxylate spot has been replaced with a more polar spot. Then, 2 equivalents of 1-(2-aminoethoxy)-2-methoxyethane are added and stirred for 6 hours at 35° C. and followed by TLC. Optionally, the temperature is increased by 10-20° C. The reaction mixture is poured into aqueous HCl/ice mixture and extracted with ethyl acetate. The extracts are back-extracted with brine, dried with sodium sulfate, filtered, stripped of solvent, and purified by silica chromatography using a DCM/MeOH gradient to yield methyl 4′-[(4-bromophenyl)[2-(2-methoxyethoxy)ethyl]-S-aminosulfonimidoyl]-[1,1′-biphenyl]-4-carboxylate

See Example 9 for synthesis of R1Q-01-R(right).

Final Assembly: R1Q-04 is synthesized from R1Q-04-R′(left) and R1Q-01-R(right) using General Procedures Z and W as in Example 2.

### Example 11—R1Q-07 Synthesis: 8-(5-{4-[4′-(methoxycarbonyl)-2-[3-(2-methoxyethoxy)prop-1-yn-1-yl]-[1,1′-biphenyl]-4-amido]phenyl}pyridine-2-amido)naphthalene-1-carboxylic acid

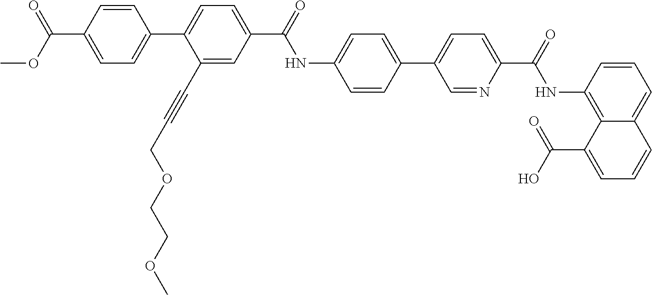 [SMILES: COCCOCC#Cc1cc(C(=O)Nc2ccc(-c3ccc(C(=O)Nc4cccc5cccc(C(=O)O)c45)nc3)cc2)ccc1-c1ccc(C(=O)OC)cc1]

### R1Q-07-R′-left synthesis: methyl 2′-bromo-4′-[(4-iodophenyl)carbamoyl]-[1,1′-biphenyl]-4-carboxylate

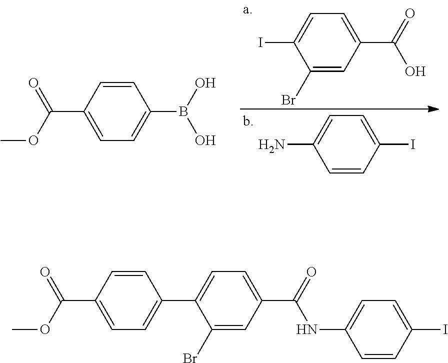

a. [4-(methoxycarbonyl)phenyl]boronic acid is combined with 3-bromo-4-iodo-benzoic acid to form 2-bromo-4′-(methoxycarbonyl)-[1,1′-biphenyl]-4-carboxylic acid using General Procedure W.

b. 2-Bromo-4′-(methoxycarbonyl)-[1,1′-biphenyl]-4-carboxylic acid and 4-iodoaniline are combined to form methyl 2′-bromo-4′-[(4-iodophenyl)carbamoyl]-[1,1′-biphenyl]-4-carboxylate using General Procedure I (see Example 2).

Final Assembly: R1Q-07 is synthesized from R1Q-07-R′(left) and R1Q-01-R(right) using General Procedures Z (1st step), W, F and X as in Example 1.

### Example 12—Synthesis of R1Q-08

### 8-(5-{4-[4′-(methoxycarbonyl)-2′-[3-(2-methoxyethoxy)prop-1-yn-1-yl]-[1,1′-biphenyl]-4-amido]phenyl}pyridine-2-amido)naphthalene-1-carboxylic acid

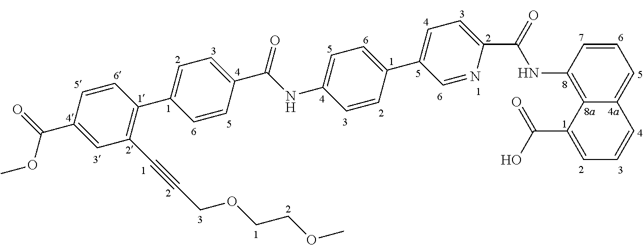 [SMILES: COCCOCC#Cc1cc(C(=O)OC)ccc1-c1ccc(C(=O)Nc2ccc(-c3ccc(C(=O)Nc4cccc5cccc(C(=O)O)c45)nc3)cc2)cc1]

### R1Q-08-R′left synthesis: methyl 2-bromo-4′-[(4-iodophenyl)carbamoyl]-[1,1′-biphenyl]-4-carboxylate

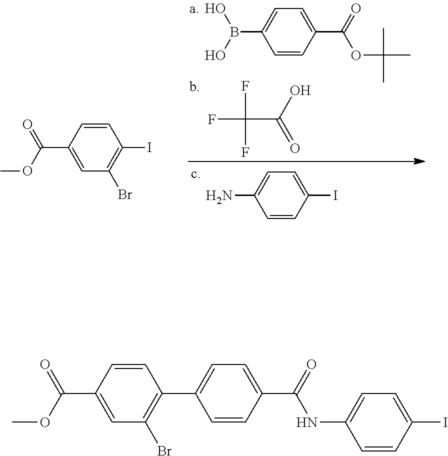

a. methyl 3-bromo-4-iodobenzoate is combined with {4-[(tert-butoxy)carbonyl]phenyl}boronic acid to form 4′-tert-butyl 4-methyl 2-bromo-[1,1′-biphenyl]-4,4′-dicarboxylate using General Procedure W.

b. General Procedure X: Conversion of t-butyl esters to carboxylic acids. Specific example: Synthesis of 2′-bromo-4′-(methoxycarbonyl)-[1,1′-biphenyl]-4-carboxylic acid Trifluoroacetic acid (30 mL) is added dropwise to a stirring slurry of 4′-tert-butyl 4-methyl 2-bromo[1,1′-biphenyl]-4,4′-dicarboxylate (20 mmol) in dichloroethane (30 mL) under N₂. The clear dark green solution is stirred at room temperature for 2.5 h, concentrated to dryness and stirred with EtOAc (100 mL) overnight. The solids are collected by filtration, washed with EtOAc and Et₂O to yield 2′-bromo-4′-(methoxycarbonyl)-[1,1′-biphenyl]-4-carboxylic acid which is used for the next step without further purification.

c. 2′-bromo-4′-(methoxycarbonyl)-[1,1′-biphenyl]-4-carboxylic acid is combined with 4-iodoaniline to form methyl 2-bromo-4′-[(4-iodophenyl)carbamoyl]-[1,1′-biphenyl]-4-carboxylate using General Procedure I (see Example 2).

R1Q-01-R(right) synthesis as in Example 9.

Final Assembly: R1Q-08 is synthesized from R1Q-08-R′(left) and R1Q-01-R(right) using General Procedures Z (1st step), W, F and X as in Example 1.

### Example 13—Synthesis of R1Q-10

### 8-[5-(4-{[1′-(methoxycarbonyl)-[4,4′-bipiperidine]-1-carbonyl]amino}phenyl)pyridine-2-amido]naphthalene-1-carboxylic acid

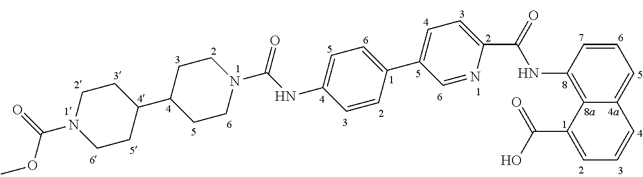 [SMILES: COC(=O)N1CCC(C2CCN(C(=O)Nc3ccc(-c4ccc(C(=O)Nc5cccc6cccc(C(=O)O)c56)nc4)cc3)CC2)CC1]

### R1Q-10-R′-left synth: Synthesis of methyl 1′-[(4-bromophenyl)carbamoyl]-[4,4′-bipiperidine]-1-carboxylate

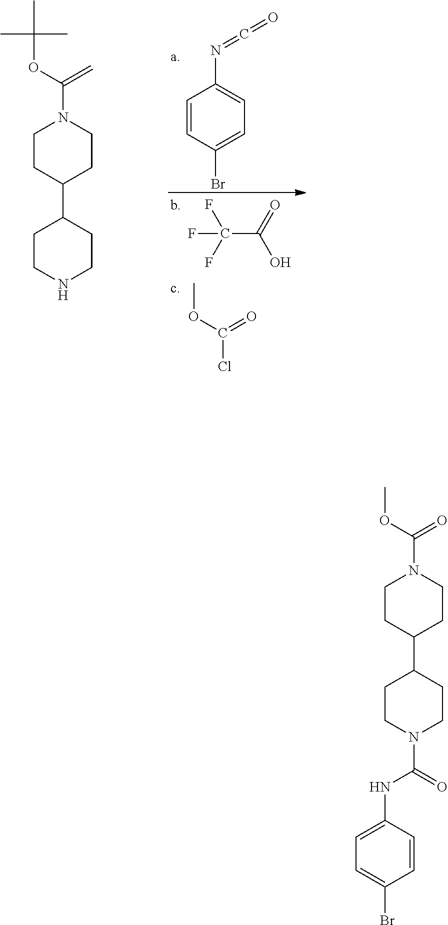

a. Synthesis of t-butyl-1′-[(4-bromophenyl)carbamoyl]-[4,4′-bipiperidine]-1-carboxylate. To a mixture of secondary amine tert-butyl [4,4′-bipiperidine]-1-carboxylate (0.45 mmol), dimethylaminopyridine (0.060 g) and triethylamine (0.2 mL, 1.4 mmol) in anhydrous tetrahydrofuran (5 mL) at 20° C. is added 4-bromo-phenyl isocyanate (0.45 mmol). After 12 h, the mixture is diluted with water (10 mL) and extracted with ethyl acetate (2×25 mL). The organic layer is washed with brine. The residue is purified by flash column chromatography on silica gel using a hexane/ethylacetate or DCM/MeOH gradient solvent mixture to yield the title compound.

b. Synthesis of N-(4-bromophenyl)-[4,4′-bipiperidine]-1-carboxamide t-Butyl-1′-[(4-bromophenyl)carbamoyl]-[4,4′-bipiperidine]-1-carboxylate is converted to N-(4-bromophenyl)-[4,4′-bipiperidine]-1-carboxamide trifluoroacetate using trifluoroacetic acid by following General Procedure X (see Example 12) Synthesis of R1Q-01R(right) is shown in Example 9.

Final Assembly: R1Q-10 is synthesized from R1Q-10-R′(left) and R1Q-01-R(right) using General Procedures Z and W as in Example 2.

### Example 14—Synthesis of R1Q-11

### 8-{5-[4-({2,2,2-trifluoro-1-[4′-(methoxycarbonyl)-[1,1′-biphenyl]-4-yl]ethyl}amino)phenyl]pyridine-2-amido}naphthalene-1-carboxylic acid

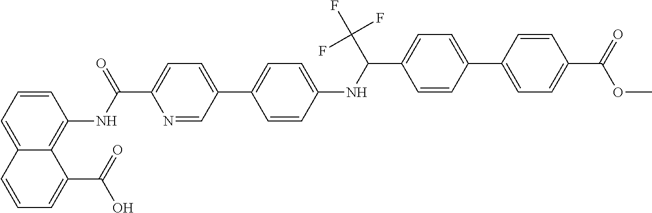 [SMILES: COC(=O)c1ccc(-c2ccc(C(Nc3ccc(-c4ccc(C(=O)Nc5cccc6cccc(C(=O)O)c56)nc4)cc3)C(F)(F)F)cc2)cc1]

R1Q-11-R′-left synthesis: 1-(4-bromophenyl)-2,2,2-trifluoroethan-1-one [4-(methoxycarbonyl)phenyl]boronic acid methyl 4′-(2,2,2-trifluoroacetyl)-[1,1′-biphenyl]-4-carboxylate

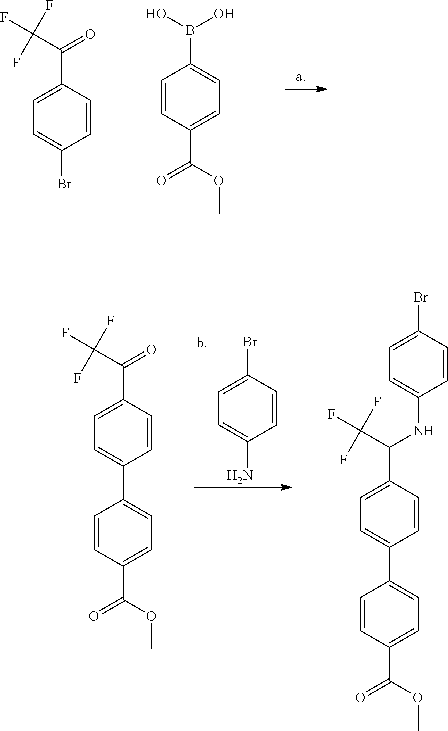 [SMILES: O=C(c1ccc(Br)cc1)C(F)(F)F ; COC(=O)c1ccc(B(O)O)cc1 ; COC(=O)c1ccc(-c2ccc(C(=O)C(F)(F)F)cc2)cc1 ; Nc1ccc(Br)cc1 ; COC(=O)c1ccc(-c2ccc(C(Nc3ccc(Br)cc3)C(F)(F)F)cc2)cc1]

a. A degassed mixture of boronic acid 38 (1.1 equiv.) 4′-bromo-2,2,2˜trifluoroacetophenone (1.0 equiv.), Ba(OH)₂ SH₂O (1.5 equiv.) Pd(PPh₃)₄ (0.03 equiv.), 1,2-dimethoxyethane and H₂O is heated for 4-6 min at 115° C. under microwave irradiation (300 W) using a CEM Discover system. The reaction mixture is cooled to room temperature, diluted with ethyl acetate, and filtered through a short pad of silica gel. The filtrate diluted with brine and extracted with ethyl acetate. The organic layer is dried over MgSO₄, the solvent is evaporated, and the residue is purified by flash column chromatography on silica gel (acetone-hexane) to give 39 in 63-78 percent yields.

b. Methyl 4′-(2,2,2-trifluoroacetyl)-[1,1′-biphenyl]-4-carboxylate and 4-bromoaniline are combined to make methyl 4′-{1-[(4-bromophenyl)amino]-2,2,2-2,2,2-trifluoroethyl}-[1,1′-biphenyl]-4-carboxylate (R1Q-11-R′-left) using. General Procedure J (see Example 20).

Synthesis of R1Q-01R(right) is shown in Example 9.

Final Assembly: R1Q-11 is synthesized from R1Q-11-R′(left) and R1Q-01-R(right) using General Procedures Z and W as in Example 2.

### Example 15—Synthesis of R1Q-13: 4-(4′-{4-[1-(methoxycarbonyl)piperidin-4-yl]benzamido}-[1,1′-biphenyl]-4-amido)naphthalene-1-carboxylic acid

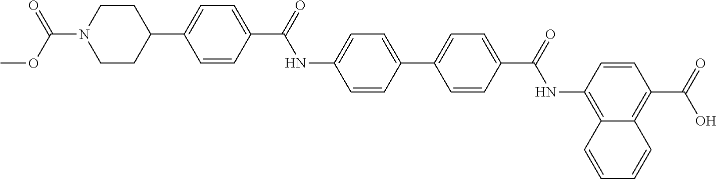 [SMILES: COC(=O)N1CCC(c2ccc(C(=O)Nc3ccc(-c4ccc(C(=O)Nc5ccc(C(=O)O)c6ccccc56)cc4)cc3)cc2)CC1]

R1Q-13-R′-left synthesis: Synthesis of methyl 4-{4-[(4-bromophenyl)carbamoyl]phenyl}piperidine-1-carboxylate

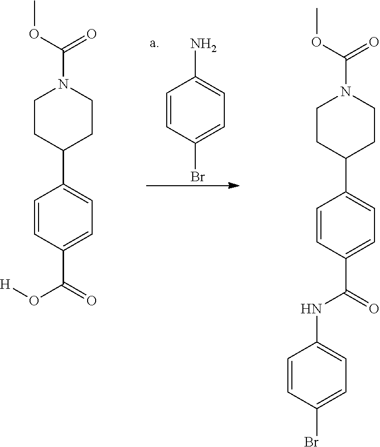 [SMILES: COC(=O)N1CCC(c2ccc(C(=O)O)cc2)CC1 ; Nc1ccc(Br)cc1 ; COC(=O)N1CCC(c2ccc(C(=O)Nc3ccc(Br)cc3)cc2)CC1]

a. 4-[1-(Methoxycarbonyl)piperidin-4-yl]benzoic acid is combined with 4-bromoaniline to form methyl 4-{4-[(4-bromophenyl)carbamoyl]phenyl} piperidine-1-carboxylate using General Procedure I (see Example 2).

b. Right Side Synthesis—see Example 5, MB-08R-(right) synthesis.

c. Final Assembly: R1Q-13 is synthesized from R1Q-13-R′(left) and MB-08-R(right) using General Procedures Z and W as in Example 2.

### Example 16—Synthesis of R1Q-15

### 4-(4′-{4′-[methoxy(methyl)carbamoyl]-[1,1′-biphenyl]-4-amido}-[1,1′-biphenyl]-4-amido)naphthalene-1-carboxylic acid

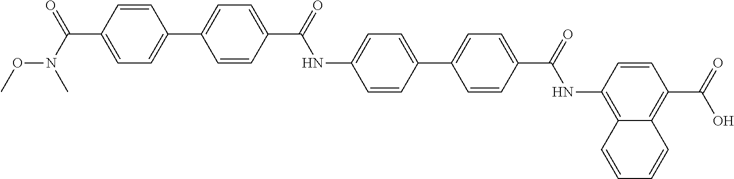 [SMILES: CON(C)C(=O)c1ccc(-c2ccc(C(=O)Nc3ccc(-c4ccc(C(=O)Nc5ccc(C(=O)O)c6ccccc56)cc4)cc3)cc2)cc1]

R1Q-15-R′-left synthesis: Synthesis of N4′-(4-bromophenyl)-N4-methoxy-N4-methyl-[1,1′-biphenyl]-4,4′-dicarboxamide

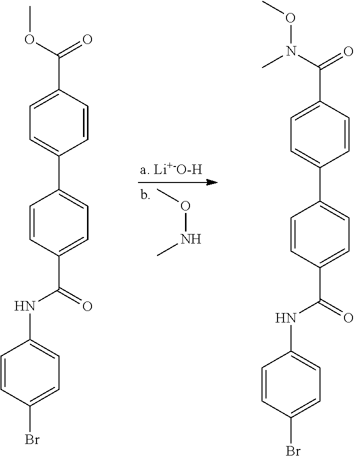

a. Methyl 4′-[(4-bromophenyl)carbamoyl]-[1,1′-biphenyl]-4-carboxylate (R2Y-01-R′(left), see example 20 for synthesis) is treated with lithium hydroxide to form 4′-[(4-bromophenyl)carbamoyl]-[1,1′-biphenyl]-4-carboxylic acid using General Procedure O (see Example 24).

b. N-methyl-methoxyamine General Procedure Y for synthesis of N-alkyl amides from carboxylic acids and amines. Specific Example: Synthesis of N4′-(4-bromophenyl)-N4-methoxy-N4-methyl-[1,1′-biphenyl]-4,4′-dicarboxamide. In a solution of 4′-[(4-bromophenyl)carbamoyl]-[1,1′-biphenyl]-4-carboxylic acid (5 mmol) in DCM (10 mL) is added carbonyldiimidazole (CDI) (5 mmol) in DCM (2 mL) at 0° C. After stirring for 0.5 h, a N,O-dimethylhydroxylamine hydrochloride (5 mmol) is added to the mixture portion wise, followed by Et₃N (5 mmol) dropwise at 0° C. The solution is stirred 1 h at 0° C. and overnight at room temperature. The reaction mixture is washed with an aqueous solution of HCl (1 N), a saturated aqueous solution of Na₂CO₃ and brine. The organic phase is dried over Na₂SO₄, filtered, and concentrated under vacuum. The crude material is purified by flash column chromatography on silica gel using a hexane/ethylacetate or DCM/MeOH gradient solvent mixture to yield the title compound.

Right side synthesis: right side synthesis is the same as MB-08R(right) synthesis

Final Assembly: R1Q-15 is synthesized from R1Q-15-R′(left) and MB-08-R(right) using General Procedures Z and W as in Example 2.

### Example 17—R1Q-16 Synthesis: 4-{4′-[4′-(dimethylcarbamoyl)-[1,1′-biphenyl]-4-amido]-[1,1′-biphenyl]-4-amido}naphthalene-1-carboxylic acid

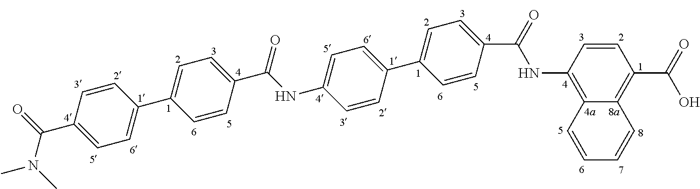 [SMILES: CN(C)C(=O)c1ccc(-c2ccc(C(=O)Nc3ccc(-c4ccc(C(=O)Nc5ccc(C(=O)O)c6ccccc56)cc4)cc3)cc2)cc1]

### R1Q-16-R′-left synthesis: Synthesis of N4′-(4-bromophenyl)-N4-dimethyl-[1,1′-biphenyl]-4,4′-dicarboxamide

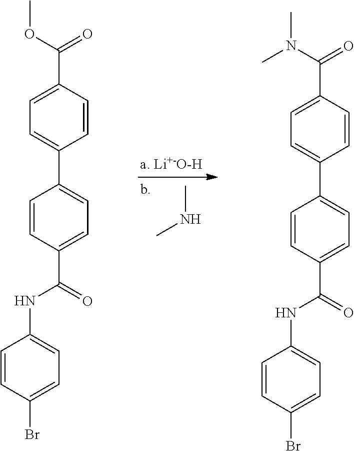

a. Methyl 4′-[(4-bromophenyl)carbamoyl]-[1,1′-biphenyl]-4-carboxylate carboxylate (R2Y-01-R′(left), see example 20 for synthesis) is treated with lithium hydroxide to form 4′-[(4-bromophenyl)carbamoyl]-[1,1′-biphenyl]-4-carboxylic acid using General Procedure O (see Example 24). b. 4′-[(4-bromophenyl)carbamoyl]-[1,1′-biphenyl]-4-carboxylic acid is combined with dimethylamine N4′-(4-bromophenyl)-N4-dimethyl-[1,1′-biphenyl]-4,4′-dicarboxamide using General Procedure Y (see Example 16).

R2Y-01-R′ Right synthesis is the same as MB-08 Synthesis is described in Example 5.17

Final Assembly: R1Q-16 is synthesized from R1Q-16-R′(left) and MB-08-R(right) using General Procedures Z and W as in Example 2.

### Example 18—Synthesis of R1Q-17: 4-(4′-{4-[5-(methoxycarbonyl)furan-2-yl]benzamido}-[1,1′-biphenyl]-4-amido)naphthalene-1-carboxylic acid

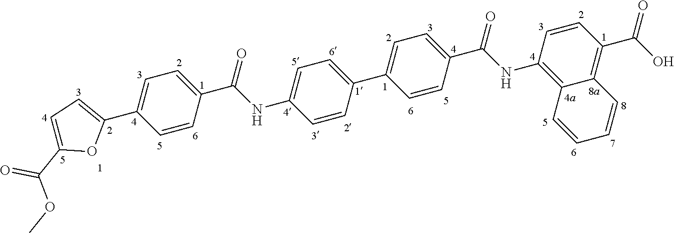 [SMILES: COC(=O)c1ccc(-c2ccc(C(=O)Nc3ccc(-c4ccc(C(=O)Nc5ccc(C(=O)O)c6ccccc56)cc4)cc3)cc2)o1]

R1Q-17-R′-left synthesis: Synthesis of 5-{4[(4-bromophenyl) carbamoyl]phenyl}furan-2-carboxylic acid

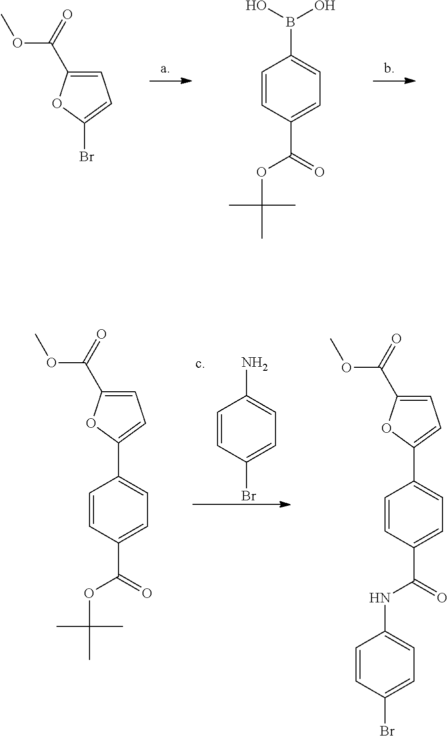

a. To a solution of methyl 5-bromofuran-2-carboxylate (4.5 mmol) in 1,4-dioxane (55 mL) is added Pd(PPh₃)₄ (264 mg, 0.22 mmol). The mixture is stirred at room temperature for 15 min, and a solution of 4-carboxyphenylboronic acid (4.78 mmol) in water (37 mL) and K₂CO₃ (1.25 g) is introduced. The mixture is stirred at 100° C. for 16 h. The reaction is filtered through a Celite pad, and the solvent is removed under vacuum. The residue is diluted in EtOAc and washed with water. The aqueous layer is acidified to pH 6 and extracted with EtOAc. The organic layer is dried over Na₂SO₄, filtered, and concentrated to dryness to afford the title product, which is used without further purification.

b. 4-[5-(Methoxycarbonyl)furan-2-yl] benzoic acid is combined with bromoaniline to form 4-methyl 5-{4[(4-bromophenyl) carbamoyl]phenyl}furan-2-carboxylate using General Procedure I (see Example 2).

Right synthesis is the same as MB-08 Synthesis is described in Example 5.

Final Assembly: R1Q-17 is synthesized from R1Q-17-R′(left) and MB-08-R(right) using General Procedures Z and W as in Example 2.

### Example 19—Synthesis of R1Q-19: 4-(4′-{3-[(2H-1,2,3,4-tetrazol-5-yl)methyl]benzamido}-[1,1′-biphenyl]-4-amido)naphthalene-1-carboxylic acid

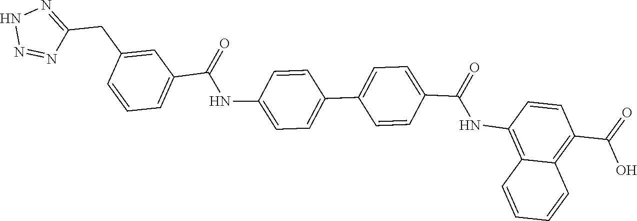 [SMILES: O=C(Nc1ccc(-c2ccc(C(=O)Nc3ccc(C(=O)O)c4ccccc34)cc2)cc1)c1cccc(Cc2nn[nH]n2)c1]

R1Q-19-R′-left synthesis: Synthesis of N-(4-bromophenyl)-3-(cyanomethyl)benzamide

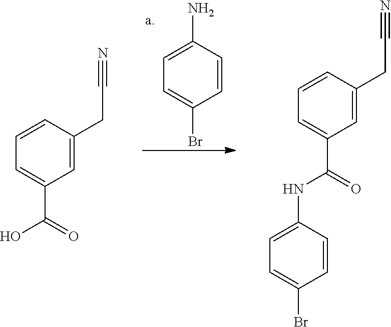 [SMILES: N#CCc1cccc(C(=O)O)c1 ; Nc1ccc(Br)cc1 ; N#CCc1cccc(C(=O)Nc2ccc(Br)cc2)c1]

a. 3-(Cyanomethyl)benzoic acid is combined with 4-bromoaniline to make N-(4-bromophenyl)-3-(cyanomethyl)benzamide using General Procedure I (see Example 2).

Right synthesis is the same as MB-08 Synthesis is described in Example 5.

R1Q-19 final assembly steps 3 and 4:

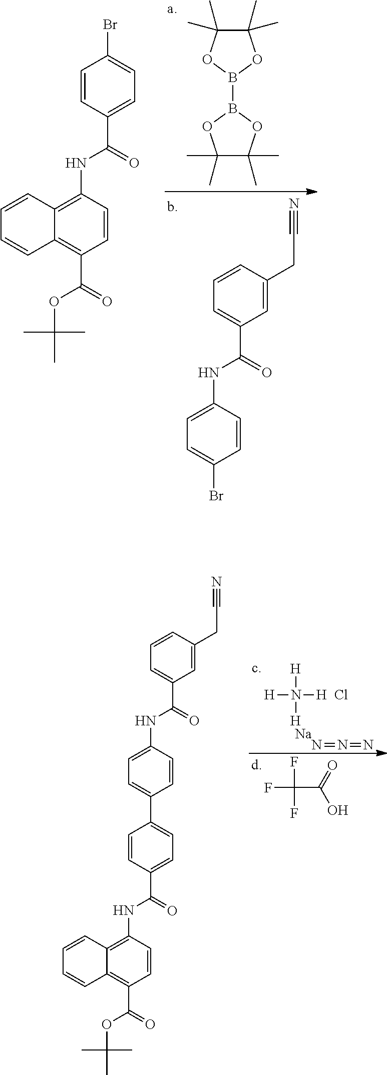

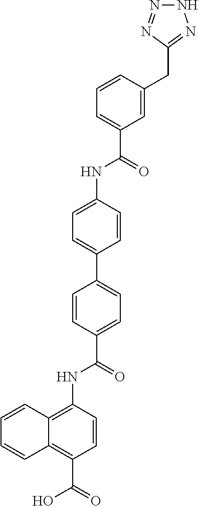 [SMILES: O=C(Nc1ccc(-c2ccc(C(=O)Nc3ccc(C(=O)O)c4ccccc34)cc2)cc1)c1cccc(Cc2nn[nH]n2)c1]

Steps a. and b. R1Q-19-R(right): tert-butyl 4-(4-bromobenzamido)naphthalene-1-carboxylate and R1Q-19-R′(left)=N-(4-bromophenyl)-3-(cyanomethyl)benzamide are converted to tert-butyl 4-{4′-[3-(cyanomethyl)benzamido]-[1,1′-biphenyl]-4-amido}naphthalene-1-carboxylate using General Procedure Z.

c. Tert-butyl 4-{4′-[3-(cyanomethyl)benzamido]-[1,1′-biphenyl]-4-amido}naphthalene-1-carboxylate is combined with sodium azide to form t-butyl-4-(4′-{3-[(2H-1,2,3,4-tetrazol-5-yl)methyl]benzamido}-[1,1′-biphenyl]-4-amido)naphthalene-1-carboxylate using General Procedure S (Example 3).

d. Tert-butyl 4-{4′-[3-(cyanomethyl)benzamido]-[1,1′-biphenyl]-4-amido}naphthalene-1-carboxylate is converted to R1Q-19=4-(4′-{3-[(2H-1,2,3,4-tetrazol-5-yl)methyl]benzamido}-[1,1′-biphenyl]-4-amido)naphthalene-1-carboxylic acid with TFA using General Procedure X (see Example 12).

### Example 19 B-R1Q-20: Synthesis of 4-(4′-(3-(2h-tetrazole-5-yl)benzamido)-{1,1′-biphenyl]-4-carboxamido}-1-napthoic acid

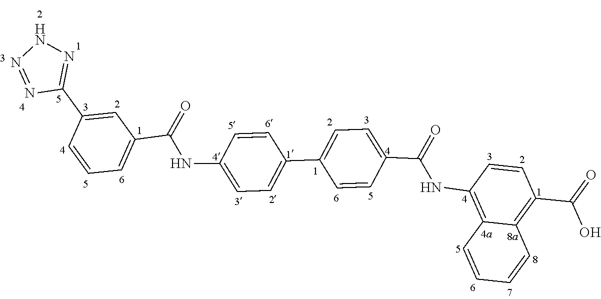 [SMILES: O=C(Nc1ccc(-c2ccc(C(=O)Nc3ccc(C(=O)O)c4ccccc34)cc2)cc1)c1cccc(-c2nn[nH]n2)c1]

R1Q-20 is synthesized from R1Q-20-R′(left) and MB-08-R(right) using General Procedures Z, S and W as in Example 19.

R1Q-20-R′(left) Synthesis of N-(4-bromophenyl)-3-cyanobenzamide. The title molecule is synthesized from 3-cyanobenzoic acid and 4-bromoaniline using General Procedure I (see Example 2).

### Example 19 C-R1Q-21: synthesis of 4-(4′-(4-(2H-tetrazol-5-yl)benzamido)-[1,1′-biphenyl]-4-carboxamido)-1-naphthoic acid

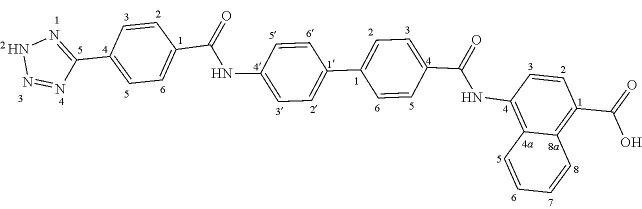 [SMILES: O=C(Nc1ccc(-c2ccc(C(=O)Nc3ccc(C(=O)O)c4ccccc34)cc2)cc1)c1ccc(-c2nn[nH]n2)cc1]

R1Q-21 is synthesized from R1Q-21-R′(left) and MB-08-R(right) using General Procedures Z, S and W as in Example 19.

R1Q-21-R′(left) {N-(4-bromophenyl)-4-cyanobenzamide} is synthesized from 4-cyanobenzoic acid and 4-bromoaniline using General Procedure I (see Example 2).

### Example 20—Synthesis of R2Y-01

### 4-[(2,2,2-trifluoro-1-{4′-[4′-(methoxycarbonyl)-[1,1′-biphenyl]-4-amido]-[1,1′-biphenyl]-4-yl}ethyl)amino]naphthalene-1-carboxylic acid

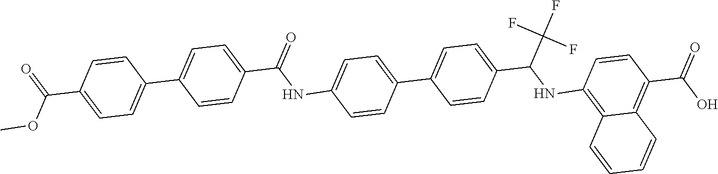 [SMILES: COC(=O)c1ccc(-c2ccc(C(=O)Nc3ccc(-c4ccc(C(Nc5ccc(C(=O)O)c6ccccc56)C(F)(F)F)cc4)cc3)cc2)cc1]

### R2Y-01-R′(left) synth: Synthesis of methyl 4′-[(4-bromophenyl)carbamoyl]-[1,1′-biphenyl]-4-carboxylate

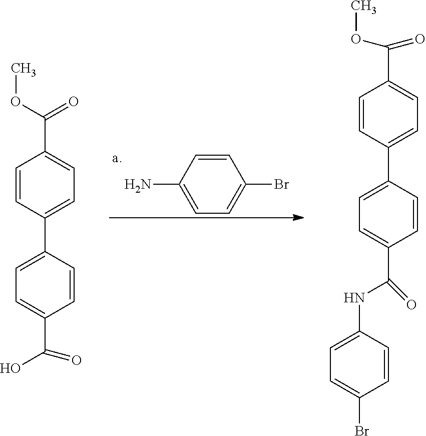 [SMILES: COC(=O)c1ccc(-c2ccc(C(=O)O)cc2)cc1 ; Nc1ccc(Br)cc1 ; COC(=O)c1ccc(-c2ccc(C(=O)Nc3ccc(Br)cc3)cc2)cc1]

a. 4′-(methoxycarbonyl)-[1,1′-biphenyl]-4-carboxylic acid and 4-bromoaniline are combined to make methyl 4′-[(4-bromophenyl)carbamoyl]-[1,1′-biphenyl]-4-carboxylate using General Procedure 1.

R2Y-01-R(right) synthesis:

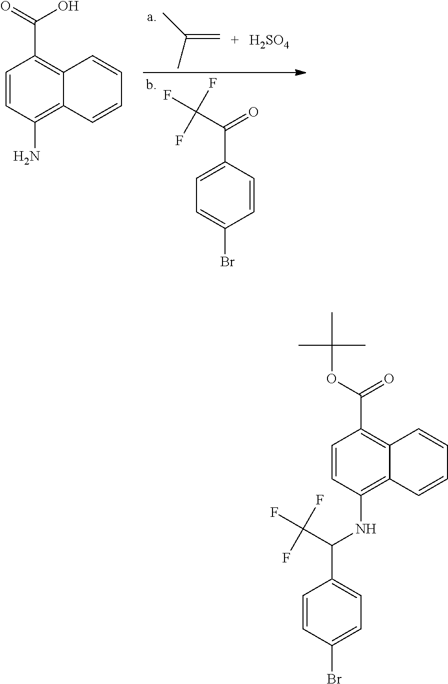

a. 4-aminonaphthalene-1-carboxylic acid is converted to t-butyl-4-aminonaphthalene-1-carboxylate using General Procedure G (see Example 1). The resulting sulfate salt is neutralized with sodium carbonate, filtered, optionally washed with water, dissolved in ethyl acetate, dried with anhydrous Na₂SO₄, filtered and stripped of solvent in vacuo. The resulting crude intermediate is optionally further purified by column chromatography on silica using methylene chloride/methanol or hexane/ethyl acetate as the solvent before proceeding to the next step.

b. General procedure J: reductive alkylation of arylamine with trifluoroacetylarene to form N-aryl-alpha-aryl-trifluoromethylamine Specific example: Synthesis of tert-butyl 4-{[1-(4-bromophenyl)-2,2,2-trifluoroethyl]amino} naphthalene-1-carboxylate. To a solution of 10.97 mmol of t-Butyl-4-aminonaphthalene-1-carboxylate in 5 mL of dichloromethane is added 0.97 mmol of 1-(4-bromophenyl)-2,2,2-trifluoroethan-1-one, 210 μL (2.9 mmol) of triethylamine, followed by 500 μL (0.5 mmol) Of TiCl₄. The mixture is stirred for 20 hours and is then diluted with methanol followed by 40 mg (1.0 mmol) of sodium borohydride. After 1 hour, the mixture is diluted with 50 mL of in aqueous NaOH and extracted with EtOAc. The combined organic layers are washed with brine, dried over sodium sulfate, and concentrated in vacuo. The crude product is purified by flash chromatography using EtOAc-hexanes (0-70 percent gradient) to afford tert-butyl 4-{[1-(4-bromophenyl)-2,2,2-trifluoroethyl]amino} naphthalene-1-carboxyl ate (R2Y-01-R(right)).

Final Assembly: R2Y-01 is synthesized from R2Y-01-R′(left) and R2Y-01-R(right) using General Procedures Z and W as in Example 2.

### Example 21—Synthesis of R2Y-04

### methyl 4′-({4′-[(8-{[4-(2-methoxyethoxy)butanamido]sulfonyl}naphthalen-1-yl)carbamoyl]-[1,1′-biphenyl]-4-yl}carbamoyl)-[1,1′-biphenyl]-4-carboxylate

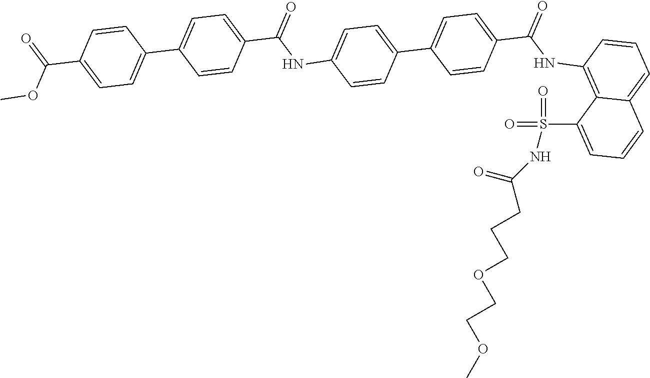 [SMILES: COCCOCCCC(=O)NS(=O)(=O)c1cccc2cccc(NC(=O)c3ccc(-c4ccc(NC(=O)c5ccc(-c6ccc(C(=O)OC)cc6)cc5)cc4)cc3)c12]

R2Y-04 left synthesis: same as R2Y-01-R′(left) Example 20:

R2Y-04-R(right)_synthesis:

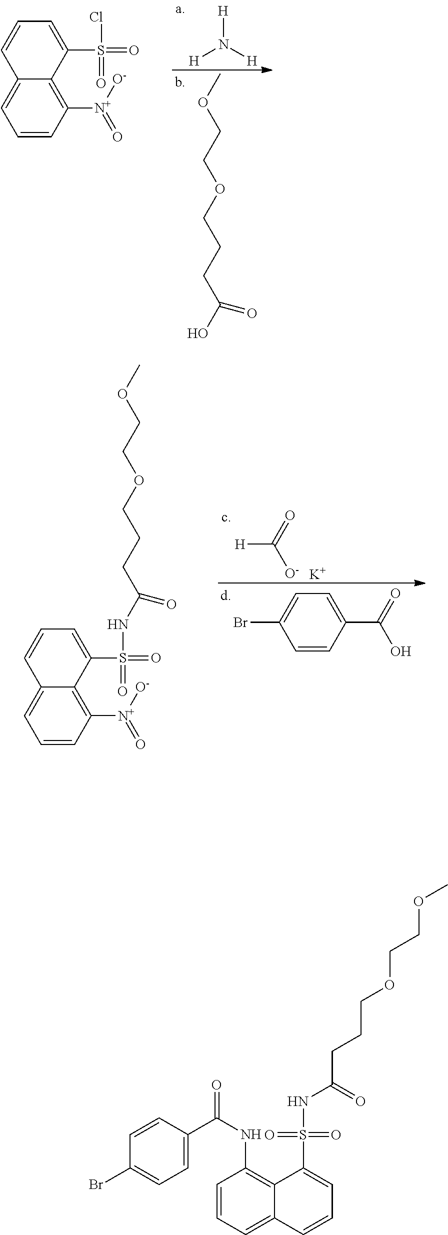

a. General Procedure K: Formation of Sulfonamide from sulfonyl chloride and ammonia Specific Example: Synthesis of 8-nitronaphthalene-1-sulfonamide ammonia

To a stirred solution of 6.5 mmol 8-nitronaphthalene-1-sulfonyl chloride and 75 mL of tetrahydrofuran at 0° C. is added 5 mL of ammonia under an inert atmosphere. The resulting suspension is stirred at ambient temperature for 15 hours and then followed by TLC until complete. Concentrated ammonium hydroxide (10 mL) and brine (10 mL) are added. The aqueous layer is adjusted to pH 7 with concentrated hydrochloric acid and is extracted with ethyl acetate. The combined organic extracts are dried over magnesium sulfate, filtered, and stripped to afford 8-nitronaphthalene-1-sulfonamide, which is optionally purified by silica chromatography using methylene chloride/methanol as the eluant.

b. General Procedure L: N-acylation of a sulfonamide Specific Example: Synthesis of N-[(8-nitronaphthalen-1-yl)sulfonyl]-4-(2-methoxyethoxy)butanamide To a stirred solution of 8-nitronaphthalene-1-sulfonamide (27.8 [mmol),] (2-methoxyethoxy)butyric acid (30.7 mmol), N,N-diisopropylethylamine (12.2 mL, 69.5 [mmol),] and DMAP (5 mole percent) in CH₂Cl₂ (275 mL) at rt under N₂ is added [BROMO-TRIPYRROLIDINO-PHOSPHONIUM] hexafluorophosphate (PyBroP) (18.1 g, 38.9 [mmol),]. The reaction mixture is allowed to stir overnight. The mixture is diluted with [1 M HCI] (100 mL) and [CH₂Cl₂] (150 mL), and the layers are separated. The organic phase is washed with 1 M HCI (1×100 mL), 1N NaOH (1×100 mL) and brine [(1]×100 mL). The organic layer is dried over Na₂SO₄, and then filtered, and the solvent is removed under reduced pressure. Purification on silica gel (EtOAc/hexane) gave N-[(8-nitronaphthalen-1-yl)sulfonyl]-4-(2-methoxyethoxy)butanamide.

c. General Procedure M: Reduction of Nitroarene to Aminorene with potassium formate. Specific Example: Synthesis of N-[(8-aminonaphthalen-1-yl)sulfonyl]-4-(2-methoxyethoxy)butanamide To a 100 mL flask are added 6.82 mmol of N-[(8-nitronaphthalen-1-yl)sulfonyl]-4-(2-methoxyethoxy)butanamide and 20 mL of ethanol under an inert atmosphere. A slurry of Pd/C (10 wt %, 50 wt % water wet, 0.17 g) in water (1 mL) is added and rinsed down with ethanol (6 mL). Potassium formate (1.74 g) is added and the slurry is warmed to 60° C. for 1 h and then refluxed for ˜1 h, following closely by TLC. The reaction is considered complete when no starting material remained relative to the intermediate N-[(8-aminonaphthalen-1-yl)sulfonyl]-4-(2-methoxyethoxy)butanamide. If a side reaction, such as cyclization to a sultam is observed (undesired), the reaction must be carried out at a lower temperatures for longer times. The product is optionally purified by silica gel chromatography using methylene chloride/methanol.

Alternately and optionally, nitroarenes can be reduced to arylamines with hydrogen using General Procedure T (see Example 3).

d. N-[(8-aminonaphthalen-1-yl)sulfonyl]-4-(2-methoxyethoxy)butanamide is combined with 4-bromobenzoic acid to form 4-bromo-N-(8-{[4-(2-methoxyethoxy)-butanamido]sulfonyl}naphthalen-1-yl)benzamide (R2Y-04-R(right)) using General Procedure I.

Final Assembly: R2Y-04 is synthesized from R2Y-01-R′(left) and R2Y-04-R(right) using General Procedure Z as in Example 2, without the deprotection step.

### Example 22—Synthesis of R2Y-07

### Methyl 4′-[(4′-{[8-(acetamidosulfonyl)naphthalen-1-yl]carbamoyl}-[1,1′-biphenyl]-4-yl)carbamoyl]-[1,1′-biphenyl]-4-carboxylate

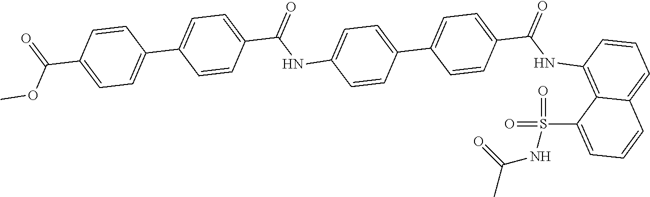 [SMILES: COC(=O)c1ccc(-c2ccc(C(=O)Nc3ccc(-c4ccc(C(=O)Nc5cccc6cccc(S(=O)(=O)NC(C)=O)c56)cc4)cc3)cc2)cc1]

R2Y-07 left synthesis: same as R2Y-01-R′(left) Example 20.

R2Y-07-R (right) synthesis:

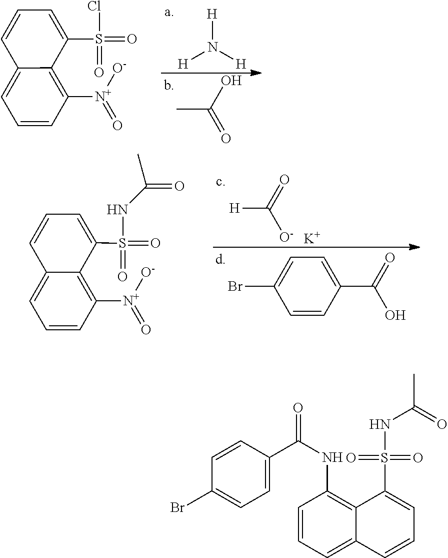

a. 8-Nitronaphthalene-1-sulfonamide is made from 8-nitronaphthalene-1-sulfonyl chloride and ammonia as in General Procedure K (see Example 21).

b. N-[(8-nitronaphthalen-1-yl)sulfonyl]acetamide is made from 8-nitronaphthalene-1-sulfonamide and acetic acid as in General Procedure L(see Example 21).

c. N-[(8-nitronaphthalen-1-yl)sulfonyl]acetamide is reduced to N-[(8-aminonaphthalen-1-yl)sulfonyl]acetamide with potassium formate as in General Procedure M (see Example 21).

d. N-[(8-aminonaphthalen-1-yl)sulfonyl]-acetamide is combined with 4-bromobenzoic acid to form 4-bromo-N-(8-(acetamidosulfonyl)naphthalen-1-yl))benzamide using General Procedure I (see Example 2).

Final Assembly: R2Y-07 is synthesized from R2Y-01-R′(left) and R2Y-07-R(right) using General Procedure Z as in Example 2, without the deprotection step.

### Example 23—Synthesis of R2Y-12

### 2-{2-[({4′-[4′-(methoxycarbonyl)-[1,1′-biphenyl]-4-amido]-[1,1′-biphenyl]-4-yl}formamido)methyl]phenyl}acetic acid

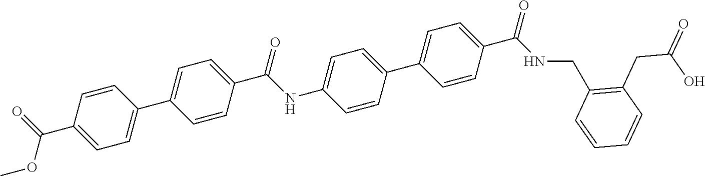 [SMILES: COC(=O)c1ccc(-c2ccc(C(=O)Nc3ccc(-c4ccc(C(=O)NCc5ccccc5CC(=O)O)cc4)cc3)cc2)cc1]

R2Y-12 left synthesis: same as R2Y-01-R′(left) Example 20.

R2Y-12 (right) synthesis:

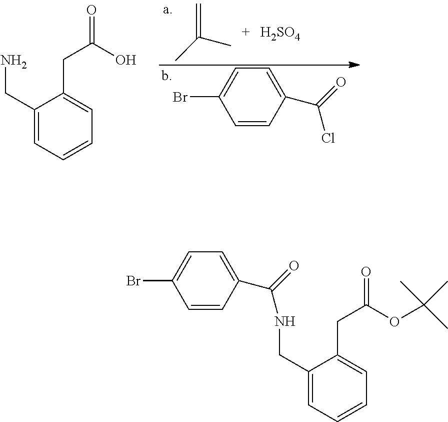

R2Y-12-R(right) synthesis:

a. t-butyl 2-[2-(aminomethyl)phenyl]acetate 2-[2-(aminomethyl)phenyl]acetic acid is converted to t-butyl 2-[2-(aminomethyl)phenyl]acetate using General Procedure G (see Example 1). The resulting sulfate salt is neutralized with sodium carbonate, filtered, optionally washed with water, dissolved in ethyl acetate, dried with anhydrous Na₂SO₄, filtered and stripped of solvent in vacuo. The resulting crude intermediate is optionally further purified by column chromatography on silica using methylene chloride/methanol or hexane/ethyl acetate as the solvent.

b. A mixture of 4-bromobenzoic acid a (2.7 mmol), t-butyl 2-[2-(aminomethyl)phenyl]acetate hydrochloride (2.7 mmol), EDC (520 mg, 2.7 mmol), and DBPEA (472 μL, 2.7 mmol) in DMF (10 mL) is stirred at RT for overnight. The mixture is partition between water (50 mL) and EtOAc (100 mL), separated, washed the aqueous layer with another portion of EtOAc (100 mL). The combined organic are washed with in HCl (50 mL), in NaOH (50 mL), dried (MgSO₄), filtered, concentrated in vacuo. The crude product is adsorbed on to Celite and purified by silica flash chromatography on a 12 g column (10-50 percent ethyl acetate-hexane) to afford tert-butyl 2-(2-{[(4-bromophenyl)formamido]methyl}phenyl)acetate.

Final Assembly: R2Y-12 is synthesized from R2Y-01-R′(left) and R2Y-12-R(right) using General Procedures Z and W as in Example 2.

### Example 24—Synthesis of R2Y-15-R

### 2-{2-[({4′-[4′-(methoxycarbonyl)-[1,1′-biphenyl]-4-amido]-[1,1′-biphenyl]-4-yl}formamido)sulfonyl]phenyl}acetic acid

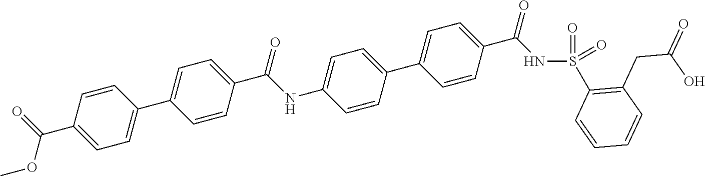 [SMILES: COC(=O)c1ccc(-c2ccc(C(=O)Nc3ccc(-c4ccc(C(=O)NS(=O)(=O)c5ccccc5CC(=O)O)cc4)cc3)cc2)cc1]

R2Y-15 left synthesis: same as R2Y-01-R′(left) Example 20.

R2Y-15-R(right) synthesis:

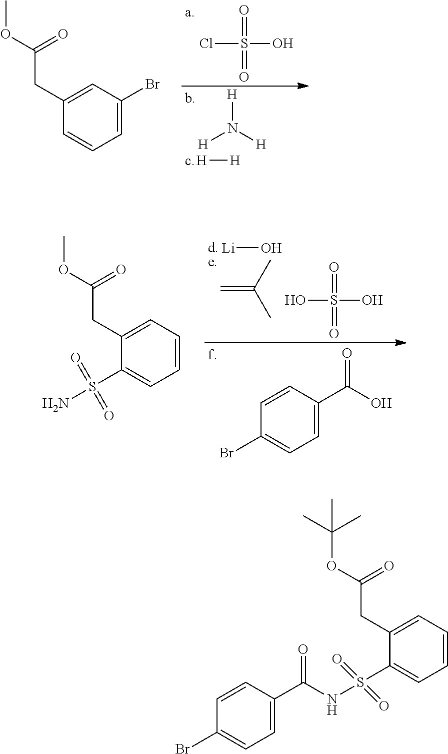

a. Chlorosulfonic acid (10 mL) is added slowly at ice-salt bath temperature to a flask containing 4 mmoles methyl 2-(3-bromophenyl)acetate. The reaction is allowed to proceed with stirring for 5 h at the same low temperature prior to pouring onto crushed ice (300 mL), and then is extracted with dichloromethane (3 150 mL). The combined dichloromethane extracts are washed with water (3×100 mL), and the organic fraction is dried (Na₂SO₄) Filtration and removal of the solvent in vacuo gives the crude methyl 2-(5-bromo-2-chlorosulfonylphenyl)acetate, which is dissolved in THF (50 mL). This solution is stirred under a stream of gaseous ammonia for 30 min at 25° C., the insoluble material is removed by filtration, and the solvent is removed from the filtrate in vacuo to yield methyl 2-(5-bromo-2-sulfamoylphenyl)acetate, which is purified by silica gel chromatography in methylene chloride/methanol.

b. General Procedure N: Reductive debromination of aryl bromide to arene. Specific Example: synthesis of methyl 2-(2-sulfamoylphenyl)acetate A solution of methyl 2-(5-bromo-2-sulfamoylphenyl) acetate (6.4 mmol) in ethanol (105 mL) is treated with triethylamine (2.68 mL, 19.2 mmol) and 20 percent palladium hydroxide on carbon (0.84 g). The mixture is hydrogenated (45-50 psi H₂) on a Parr shaker for 6.5 hours at room temperature, then filtered through a celite pad to remove the catalyst which is washed with additional ethanol (3×5 mL). The filtrate and washings are evaporated under vacuum to a residue which is partitioned between ethyl acetate (60 mL) and 1N hydrochloric acid (50 mL). The organic phase is washed with brine (25 mL), dried over sodium sulfate, filtered, and evaporated under vacuum to afford methyl 2-(2-sulfamoylphenyl)acetate, which is used without further purification.

c. General Procedure O: Selective hydrolysis of methyl ester to carboxylic acid Specific Example: Synthesis of 2-(2-sulfamoylphenyl)acetic acid A solution of methyl 2-(2-sulfamoylphenyl)acetate (4.72 mmol) in a mixture of methanol (4.5 mL), tetrahydrofuran (4 mL) and water (1.5 mL) is treated with lithium hydroxide monohydrate (0.5 g, 11.9 mmol) and the resulting reaction mixture is stirred at ambient temperature for 1 h. The precipitated solid in the reaction mixture is filtered and washed well with diethyl ether. Toluene (10 mL) is added and removed at low pressure to azeotrope out remaining traces of water, and the residue is dried under high vacuum overnight. The crude 2-(2-sulfamoylphenyl)acetic acid is used for the next step without further purification.

d. 2-(2-Sulfamoylphenyl)acetic acid is converted to t-butyl 2-(2-sulfamoylphenyl)acetate using General Procedure G (see Example 1).

e. t-Butyl 2-(2-sulfamoylphenyl)acetate and 4-bromobenzoic acid are combined to make tert-butyl 2-({[(4-bromophenyl)formamido]sulfonyl}phenyl)acetate using General Procedure L (see Example 21).

Final Assembly: R2Y-15 is synthesized from R2Y-01-R′(left) and R2Y-15-R(right) using General Procedures Z and W as in Example 2.

### Example 25—Synthesis of W-03

### 4-(4-{6-[4′-(methoxycarbonyl)-[1,1′-biphenyl]-4-amido]pyridin-3-yl}benzamido)naphthalene-1-carboxylic acid

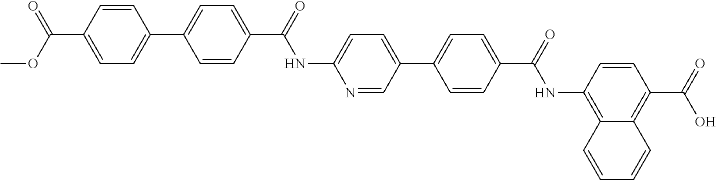 [SMILES: COC(=O)c1ccc(-c2ccc(C(=O)Nc3ccc(-c4ccc(C(=O)Nc5ccc(C(=O)O)c6ccccc56)cc4)cn3)cc2)cc1]

W-03-R′-left synthesis:

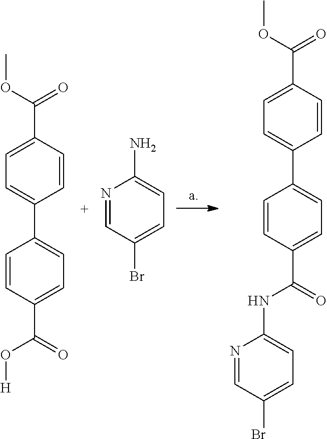

### W-03-R-left synth: Synthesis of methyl 4′-[(5-bromopyridin-2-yl)carbamoyl]-[1,1′-biphenyl]-4-carboxylate

a. Methyl 4′-[(4-bromophenyl)carbamoyl]-[1,1′-biphenyl]-4-carboxylate is combined with 2-amino-5-bromopyridine to form methyl 4′-[(5-bromopyridin-2-yl)carbamoyl]-[1,1′-biphenyl]-4-carboxylate using General Procedure I (see Example 2). W-03-R′ right synthesis: W-O3-R′ right synthesis is the same as MB-08 Synthesis as described in Example 5.

Final Assembly: W-03 is synthesized from W-03-R′(left) and MB-08-R(right) using General Procedures Z and W as in Example 2.

### Example 26—Synthesis of W-04: 4-(4-(5-(4′-(methoxycarbonyl)-[1,1′-biphenyl]-4-carboxamido)thiophen-2-yl)benzamido)-1-naphthoic acid

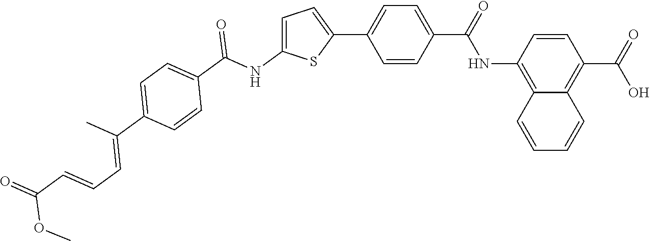 [SMILES: COC(=O)/C=C/C=C(\C)c1ccc(C(=O)Nc2ccc(-c3ccc(C(=O)Nc4ccc(C(=O)O)c5ccccc45)cc3)s2)cc1]

W-04 is synthesized from W-04-R′(left) and MB-08-R(right) using General Procedures Z and W as in Example 2.

### Example 27—Synthesis of W-06

### 4-(5-{4-[4′-(methoxycarbonyl)-[1,1′-biphenyl]-4-amido]phenyl}thiophene-2-amido)naphthalene-1-carboxylic acid

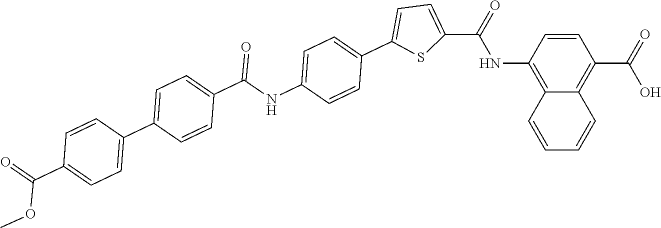 [SMILES: COC(=O)c1ccc(-c2ccc(C(=O)Nc3ccc(-c4ccc(C(=O)Nc5ccc(C(=O)O)c6ccccc56)s4)cc3)cc2)cc1]

W-06 left synthesis: W-06 left synthesis the same as R2Y-01-R′(left) Example 20.

W-06-R(right) synthesis:

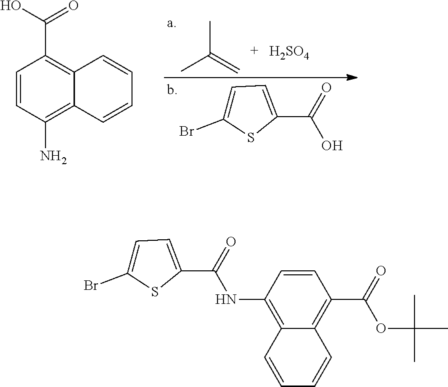

a. 4-Aminonaphthalene-1-carboxylic acid and isobutene are converted to t-butyl 4-aminonaphthalene-1-carboxylate using General Procedure G (see Example 1). b. t-Butyl 4-aminonaphthalene-1-carboxylate is combined with 5-bromothiophene-2-carboxylic acid to form tert-butyl 4-(5-bromothiophene-2-amido)-naphthalene-1-carboxylate using General Procedure I (see Example 2).

Final Assembly: W-06 is synthesized from R2Y-01-R′(left) and W-06-R(right) using General Procedures Z and W as in Example 2.

### Example 28—Synthesis of W-07

### 4-{N-[2-(2-methoxyethoxy)ethyl]4′-[4′-(methoxycarbonyl)-[1,1′-biphenyl]-4-amido]-[1,1′-biphenyl]-4-sulfonoimidamido}naphthalene-1-carboxylic acid

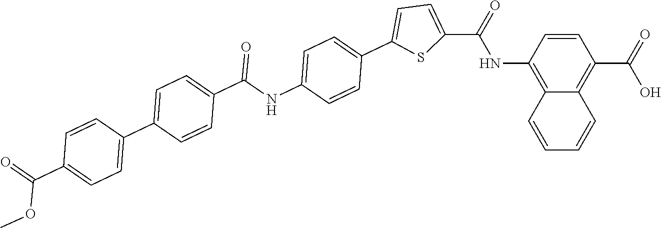 [SMILES: COC(=O)c1ccc(-c2ccc(C(=O)Nc3ccc(-c4ccc(C(=O)Nc5ccc(C(=O)O)c6ccccc56)s4)cc3)cc2)cc1]

W-07 left synthesis: W-07 left synthesis same as R2Y-01.

W-07-R(right) synthesis:

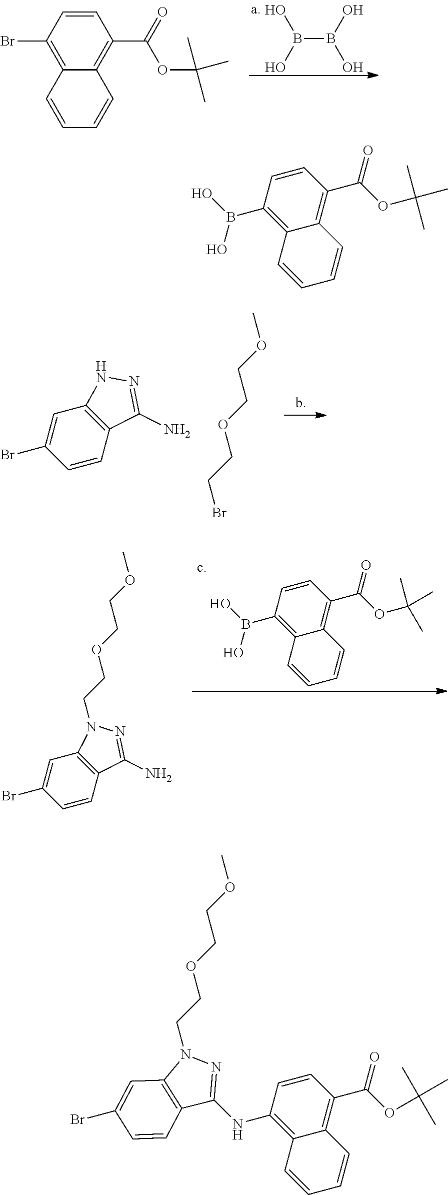

a. Synthesis of ({4-[(tert-butoxy)carbonyl]naphthalen-1-yl}boronic acid) tert-butyl 4-bromonaphthalene-1-carboxylate (13.5 mmol), hypodiboric acid (3.6 g, 40.4 mmol), 2-lohexylphosphino)-2′,4′,6′-triisopropylbiphenyl (XPhos) (260 mg, 540 μmol), Pd-XPhos 30 mg, 269. μmol) and potassium acetate (7.9 g, 80.7 mmol) are placed in a 2000 mL pressure vessel. Then EtOH (200 mL), THF (100 mL) and ethane-1,2-diol (4.5 mL, 80.7 mmol) are added, and the reaction mixture is degassed (3×, vacuum/N2). The pressure vial is capped and the reaction mixture is stirred at 80° C. for 16 h. Additional amounts of hypodiboric acid mg, 40.4 mmol), Pd-XPhos G3 (230 mg, 269. μmol), 2-(dicyclohexylphosphino)-2′,4′,6′-propylbiphenyl (XPhos) (257 mg, 538 μmol) and potassium acetate (7.9 g, 80.7 mmol) are added, and the reaction mixture is degassed (3× vacuum/N2). Then, ethane-1,2-diol 500 mL, 80.7 mmol) is added, the reaction mixture is degassed again, and stirred at 100° C. for 4 h. The reaction mixture is diluted with THF (200 mL), filtered (to remove Pd-black), and concentrated under reduced pressure. The ({4-[(tert-butoxy)carbonyl]naphthalen-1-yl}boronic acid) is used for step c without further purification.

b. Synthesis of 6-bromo-1-[2-(2-methoxyethoxy)ethyl]-1H-indazol-3-amine In a round bottom flask, DMSO (50 ml) and ground KOH powder (1.365 g, 24.3 mmoles.) are added and stirred for 5 minutes at room temperature. To this 6-bromo-1H-indazol-3-amine (12.15 mmoles) is added in one portion. After 5 minutes, 1-bromo-2-(2-methoxyethoxy)ethane chloride (12.8 mmoles) is added using DMSO (25 ml) solvent over a period of 20-30 minutes. After stirring the reaction mixture for additional one hour, it is checked by TLC. Additional heating and stirring are used as needed. The reaction mixture is diluted with water and extracted the compound with dichloro methane (3×20 ml). The combined organic layers are washed with brine and passed through dry Na₂SO₄. Evaporation of the solvent and silica gel column purification using MeOH:CH₂Cl₂ afforded 6-bromo-1-[2-(2-methoxyethoxy)ethyl]-1H-indazol-3-amine

c. Synthesis of tert-butyl 4-({6-bromo-1-[2-(2-methoxyethoxy)ethyl]-1H-indazol-3-yl}amino)naphthalene-1-carboxylate Cu(OAc)₂ powder (1.53 g, 8.45 mmol, 1.2 equiv) is added to a round bottom flask, to which dichloromethane (15 ml) and MeOH (1.0 ml) solvents are added. The mixture is stirred for 5 minutes, after which 6-bromo-1-[2-(2-methoxyethoxy)ethyl]-1H-indazol-3-amine (7.04 mmol, 1.0 equiv) is added in one portion. To this mixture, ({4-[(tert-butoxy)carbonyl]naphthalen-1-yl}boronic acid) (14.1 mmol, 2.0 equiv) and di-isopropyl ethyl amine (1.5 ml, 8.45 mmol, 1.2 equiv) are added one after the other. This mixture is stirred at room temperature for 20 hrs, after which 6N NH₃ in methanol solution is added and stirred for additional 2 hrs. Then it is passed through a bed of silica gel and washed couple of times with dichloro methane solvent. The organic layer is washed with tartarate and brine solution. The crude mixture is dried over Na₂SO₄ and concentrated. Silica chromatography using MeOH/DCM solvent system affords tert-butyl 4-({6-bromo-1-[2-(2-methoxyethoxy)ethyl]-1H-indazol-3-yl}amino)naphthalene-1-carboxyl ate (W-07-R(right)).

Final Assembly: W-07 is synthesized from R2Y-01-R′(left) and W-07-R(right) using General Procedures Z and W as in Example 2.

### REFERENCES FOR FORMULA I COMPOUNDS

Rogers, Steven A.; Huigens III, Robert W.; Melander, Christian—Journal of the American Chemical Society, 2009, vol. 131, #29, p. 9868-9869
Lee, Jinbo; Smith, Michael J.; Moretto, Alessandro Fabio; Wan, Zhao-Kui; Binnun, Eva Deanna; Xu, Weixin; Foreman, Kenneth W.; Joseph-McCarthy, Diane M.; Erbe, David V.; Tam, Steve Y.—US Publication No. US2006/135488
ELI LILLY AND COMPANY; BLANCO-PILLADO, Maria-Jesus; VETMAN, Tatiana Natali; FISHER, Matthew Joseph; KUKLISH, Steven Lee—International Publication No. WO2014/04230
Bergman, Jan; Stensland, Birgitta—Journal of Heterocyclic Chemistry, 2014, vol. 51, #1, pp. 1-10];
BOEHRINGER INGELHEIM INTERNATIONAL GMBH; BOEHRINGER INGELHEIM PHARMA GMBH and CO. KG—International Publication No. WO2008/70507
Zhang et al, ACS Catal. 2016, V6, pp 6229-6235;
E. I. DuPont De Nemours and Company—U.S. Pat. No. 4,746,356;
JANSSEN PHARMACEUTICA, N.V. International Publication No. WO2004/007463, location in patent: Page 172
Warner-Lambert Company—U.S. Pat. No. 4,582,909
Tacconi, et al, Journal of Chemical Research, Miniprint, 1980, #1, p. 201-216 and Warner-Lambert Company—U.S. Pat. No. 4,582,909
Miller, Ross A.; Humphrey, Guy R.; Lieberman, David R.; Ceglia, Scott S.; Kennedy, Derek J.; Grabowski, Edward J. J.; Reider, Paul J.—Journal of Organic Chemistry, 2000, vol. 65, #5, p. 1399-1406];
Luts, H. A.—Journal of Organic Chemistry, 1968, vol. 33, pp. 4528-4529
Zhang, et al., Chemical Communications (Cambridge, United Kingdom), 2012, V 48, pp. 10672 10674
Chen, Rao, Knaus, Bioorganic and Medicinal Chemistry, 2005, vol. 13, #15, p. 4694-4703;
Reductive dehalogenation of ArBr to ArH Merck and Co., Inc.—U.S. Pat. No. 5,994,345
BRISTOL-MYERS SQUIBB COMPANY; SMITH II, Leon M. et al. International Publication No. WO2017/123860
LEXICON PHARMACEUTICALS, INC.—International Publication No WO2008/089307
Cheng et al., Bioorg. Med. Chem. Lett., 2018, V 28, pp. 1330-1335
Pandarus et al. Beilstein Journal of Organic Chemistry 2014, V10, pp 897-901;
Masanori et al., Synthetic Communications V37, pp 667-674 (2007)
De Oliveira Lopes et al., Journal of Molecular Catalysis B: Enzymatic, 2014, V104, pp. 101-107;
Zhang et al., ACS Catal. 2016, V6, pp 6229-6235
Igeta et al.—Chemical and Pharmaceutical Bulletin, 1970, vol. 18, p. 1228
Igeta et al., Tet Lett. 1969 p 2359 reductive coupling of 3-amino-6-chloropyridazine; Rogers, Steven A.; Huigens III, Robert W.; Melander, Christian—Journal of the American Chemical Society, 2009, vol. 131, pp. 9868-9869

### Synthesis of Formula II Compounds

### Example 31—Synthesis of MB-17

### 8-[4′-({5-[(2H-1,2,3,4-tetrazol-5-yl)methyl]-2H-1,2,3,4-tetrazol-2-yl}methyl)-[1,1′-biphenyl]-4-amido]naphthalene-1-carboxylic acid

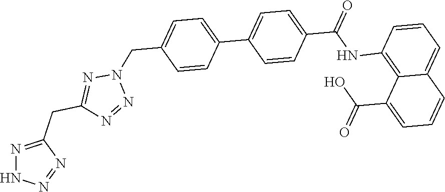 [SMILES: O=C(Nc1cccc2cccc(C(=O)O)c12)c1ccc(-c2ccc(Cn3nnc(Cc4nn[nH]n4)n3)cc2)cc1]

a. MB-17-R′-left—5-[(2H-1,2,3,4-tetrazol-5-yl)methyl]-2H-1,2,3,4-tetrazole is commercially available.

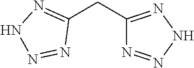 [SMILES: C(c1nn[nH]n1)c1nn[nH]n1]

b. MB-17-Y-Core: 5-[(2H-1,2,3,4-tetrazol-5-yl)methyl]-2H-1,2,3,4-tetrazole is commercially available from Chemical Block—catalog #BB ZERO/011216

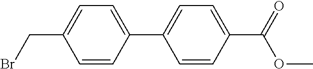 [SMILES: COC(=O)c1ccc(-c2ccc(CBr)cc2)cc1]

c. MB-17-R-right synthesis:

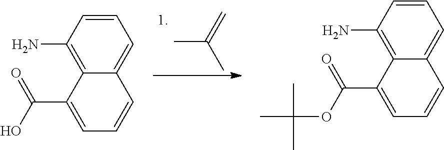

8-amino-naphtalene-1-carboxylic acid and isobutene are combined to form t-butyl-8-amino-naphtalene-1-carboxylate using General Procedure G (see Example 1).

d. MB-17 final assembly:

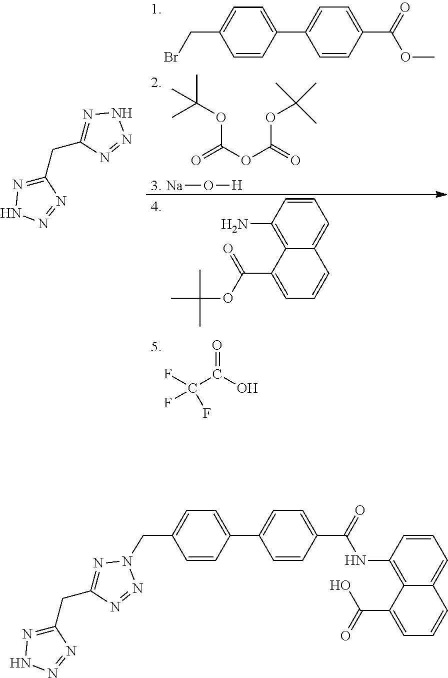

The precursors for MB-17 final assembly are MB-17-R′-left, MB-17-Y(core), and MB-17-R-right.

1. General Procedure E: alkylation of 5-substituted tetrazole selectively at 2-position using a benzylic halide. Specific example: synthesis of methyl 4′-({5-[(2H-1,2,3,4-tetrazol-5-yl)methyl]-2H-1,2,3,4-tetrazol-2-yl}methyl)-[1,1′-biphenyl]-4-carboxylate 1 equivalent of methyl 4′-(bromomethyl)-[1,1′-biphenyl]-4-carboxylate is added dropwise to a solution of 5-[(2H-1,2,3,4-tetrazol-5-yl)methyl]-2H-1,2,3,4-tetrazole and 1 equivalent of DIPEA in THF or another polar aprotic solvent until formation of monoadduct reaches a maximum.

2. General Procedure D. Boc protection of nitrogen in amines and N-heterocycles. Specific example: Di-tert-butyl decarbonate is combined with methyl 4′-({5-[(2H-1,2,3,4-tetrazol-5-yl)methyl]-2H-1,2,3,4-tetrazol-2-yl}methyl)-[1,1′-biphenyl]-4-carboxylate to form 4′-{[5-({2-[(tert-butoxy)carbonyl]-2H-1,2,3,4-tetrazol-5-yl}methyl)-2H-1,2,3,4-tetrazol-2-yl]methyl}-4-methoxycarbonyl-[1,1′-biphenyl] tert-butyl 5-[(2-{[4′-(methoxycarbonyl)-[1,1′-biphenyl]-4-yl]methyl}-2H-1,2,3,4-tetrazol-5-yl)methyl]-2H-1,2,3,4-tetrazole-2-carboxylate;

3. tert-butyl 5-[(2-{[4′-(methoxycarbonyl)-[1,1′-biphenyl]-4-yl]methyl}-2H-1,2,3,4-tetrazol-5-yl) methyl]-2H-1,2,3,4-tetrazole-2-carboxylate is converted to 5-[(2-{[4′-({8-[(tert-butoxylcarbonyl]naphthalen-1-yl} carbamoyl)-[1,1′-biphenyl]-4-yl]methyl}-2H-1,2,3,4-tetrazol-5-yl)methyl]-2H-1,2,3,4-tetrazole-2-carboxylic acid using General Procedure O (see Example 24).

4. Tert-butyl 8-aminonaphthalene-1-carboxylate and 5-[(2-{[4′-({8-[(tert-butoxy) carbonyl] naphthalen-1-yl} carbamoyl)-[1,1′-biphenyl]-4-yl]methyl}-2H-1,2,3,4-tetrazol-5-yl)methyl]-2H-1,2,3,4-tetrazole-2-carboxylic acid are combined to form tert-butyl 5-[(2-{[4′-({8-[(tert-butoxy)carbonyl] naphthalen-1-yl}carbamoyl)-[1,1′-biphenyl]-4-yl]methyl}-2H-1,2,3,4-tetrazol-5-yl)methyl]-2H-1,2,3,4-tetrazole-2-carboxylate using General Procedure I.

5. tert-butyl 5-[(2-{[4′-({8-[(tert-butoxy)carbonyl]naphthalen-1-yl}carbamoyl)-[1,1′-biphenyl]-4-yl]methyl}-2H-1,2,3,4-tetrazol-5-yl)methyl]-2H-1,2,3,4-tetrazole-2-carboxylate is converted to 8-[4′-({5-[(2H-1,2,3,4-tetrazol-5-yl)methyl]-2H-1,2,3,4-tetrazol-2-yl}methyl)-[1,1′-biphenyl]-4-amido] naphthalene-1-carboxylic acid using General Procedure X (see Example 12).

### Example 33—Synthesis of MB-19

### 5-{4-[hydroxy(1-{2-hydroxy-5-[methoxy(methyl)carbamoyl]phenyl}-1H-1,2,3-triazol-4-yl)methyl]-1H-1,2,3-triazol-1-yl}naphthalene-1-carboxylic acid

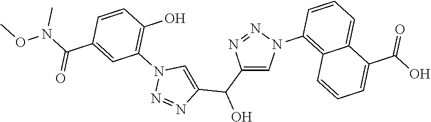 [SMILES: CON(C)C(=O)c1ccc(O)c(-n2cc(C(O)c3cn(-c4cccc5c(C(=O)O)cccc45)nn3)nn2)c1]

MB-19-R′-left synthesis of 3-azido-4-hydroxy-N-methoxy-N-methylbenzamide

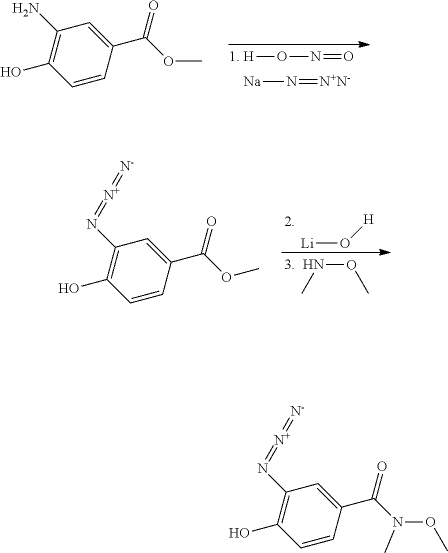

Synthesis of methyl 3-azido-4-hydroxy-benzoate. 1. A solution of methyl (4-hydroxy-3-amino)-benzoate and sodium nitrite is treated with hydrochloric acid to increase the pH to 2. Then, 1.5 equivalent of sodium azide is added. After nitrogen evolution has ceased, the reaction mixture is stripped of solvent, and the product is dissolved in ethyl acetate and washed with water 3× to remove salts. The methyl 3-azido-4-hydroxy-benzoate is purified using silica gel chromatography with ethyl acetate/hexanes.

2. Synthesis of 3-azido-4-hydroxy-benzoic acid. Methyl 3-azido-4-hydroxy-benzoate is combined with lithium hydroxide (1 equivalent) in methanol/THF for 1 hour at 0° C. The solution is made acidic with concentrated HCl to precipitate the 3-azido-4-hydroxy-benzoate. The crude material is isolated as the residue by filtration, washed with water and ether, and dried in vacuo.

3. Synthesis of MB-19-R′-left=3-azido-4-hydroxy-N-methoxy-N-methylbenzamide. Without further purification, the 3-azido-4-hydroxy-benzoate is combined with EDC, DMAP and methoxy(methyl)amine and stirred until the reaction is complete by TLC. The reaction mixture is dissolved in ethyl acetate, washed with 1N aqueous HCl 3× to remove DMAP and amines, stripped of solvent, and purified using silica gel chromatography with ethyl acetate/hexanes, and stripped of solvent to yield the title product 3-azido-4-hydroxy-N-methoxy-N-methylbenzamide (MB-19-R′-left).

MB-19-R′-left is a precursor for MB-19 and MB-23.

### MB-19-Y(core): 3-hydroxy-penta-1,4-diyne is commercially available (CAS 56598-53-9)

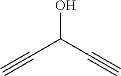

MB-19-R-right synthesis:

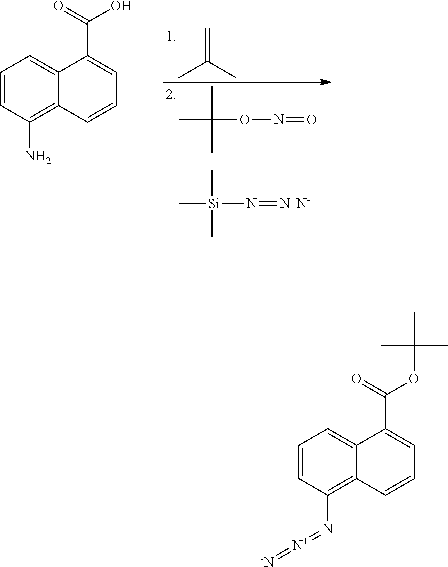

1. 5-Amino-naphtalene-1-carboxylic acid and isobutene are combined to form t-butyl-5-amino-naphtalene-1-carboxylate using General Procedure G (see Example 1).
2. T-butyl-5-amino-naphtalene-1-carboxylate is combined with t-butyl nitrite followed by trimethylsilyl azide to form t-butyl-5-azidonapthalene-1-carboxylate.

MB-19 final assembly: Synthesis of 5-{4-[hydroxy(1-{2-hydroxy-5-[methoxy(methyl)carbamoyl]phenyl}-1H-1,2,3-triazol-4-yl)methyl]-1H-1,2,3-triazol-1-yl}naphthalene-1-carboxylic acid:

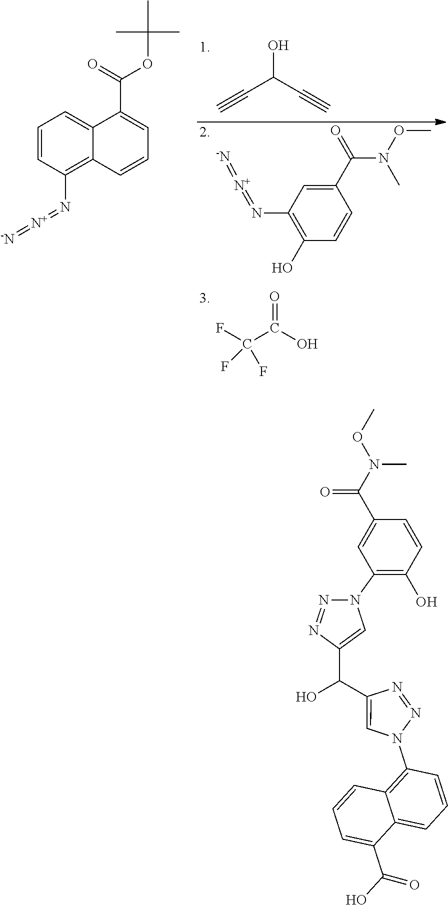

1. t-Butyl-5-azidonapthalene-1-carboxylate (1 equivalent) is added dropwise to 3-hydroxy-penta-1,4-diyne to form the monoadduct 4-hydroxy-3-[4-(1-hydroxyprop-2-yn-1-yl)-1H-1,2,3-triazol-1-yl]-N-methoxy-N-methylbenzamide using General procedure U (see Example 6).

2. 4-hydroxy-3-[4-(1-hydroxyprop-2-yn-1-yl)-1H-1,2,3-triazol-1-yl]-N-methoxy-N-methylbenzamide is combined with 3-azido-4-hydroxy-N-methoxy-N-methylbenzamide to form tert-butyl 5-{4-[hydroxy(1-{2-hydroxy-5-[methoxy(methyl)carbamoyl]phenyl}-1H-1,2,3-triazol-4-yl)methyl]-1H-1,2,3-triazol-1-yl}naphthalene-1-carboxylate using General Procedure U (see Example 6).

3. tert-butyl 5-{4-[hydroxy(1-{2-hydroxy-5-[methoxy(methyl)carbamoyl]phenyl}-1H-1,2,3-triazol-4-yl)methyl]-1H-1,2,3-triazol-1-yl}naphthalene-1-carboxylate is converted to 5-{4-[hydroxy(1-{2-hydroxy-5-[methoxy(methyl)carbamoyl]phenyl}-1H-1,2,3-triazol-4-yl)methyl]-1H-1,2,3-triazol-1-yl}naphthalene-1-carboxylic acid (MB-19) using general procedure X

### Example 34—Synthesis MB-20

### 5-[4-(5-{1-[2-hydroxy-5-(methoxycarbonyl)phenyl]-1H-1,2,3-triazol-4-yl}pyridin-3-yl)-1H-1,2,3-triazol-1-yl]naphthalene-1-carboxylic acid

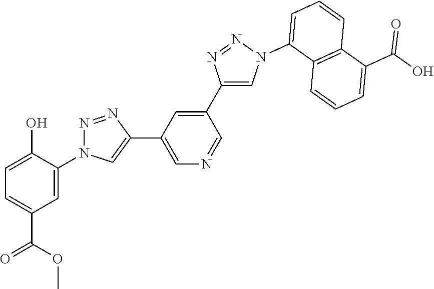 [SMILES: COC(=O)c1ccc(O)c(-n2cc(-c3cncc(-c4cn(-c5cccc6c(C(=O)O)cccc56)nn4)c3)nn2)c1]

### MB-20-R′-left synthesis of methyl 3-azido-4-hydroxybenzoate

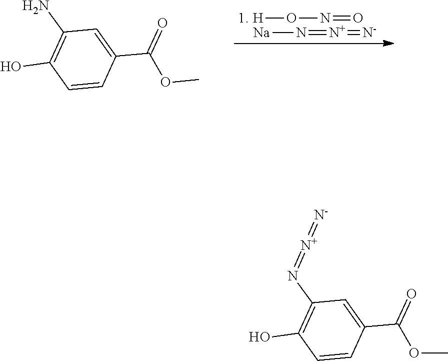

MB-20-Y(core):

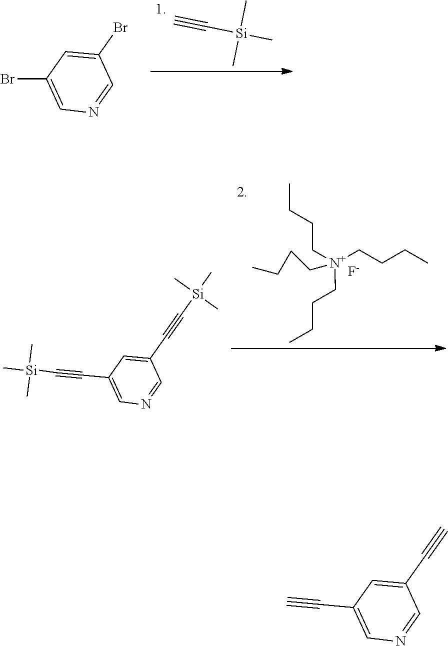

3,5-dibromopyridine and 2 equivalents of trimethylsilylethyne are converted to 3,5-bis(trimethylsilylethynyl) pyridine using General Procedure F (see Example 1). 3,5-Bis(trimethylsilylethynyl) pyridine is converted to MB-20-Y(core)=3,5-diethynylpyridine using TBAF.

c. For the synthesis of MB-19-R-right, see Example 33.

d. MB-20 final assembly. The synthetic procedure in example 33 is used, but the precursors for MB-20 are MB-20-R′-left, MB-20-Y(core), and MB-19-R-right.

### Example 35—Synthesis of MB-22

### 3-{[5-({2-[(2-butanamido-5-nitrophenyl)methyl]-2H-1,2,3,4-tetrazol-5-yl}methyl)-2H-1,2,3,4-tetrazol-2-yl]methyl}naphthalene-1-carboxylic acid

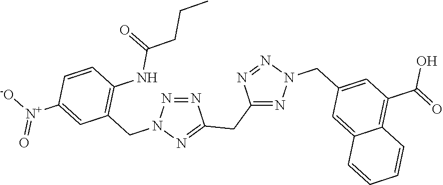

a. MB-22-R′-left: 2-(bromomethyl)-1-fluoro-4-nitrobenzene is commercially available: CAS #454-15-9

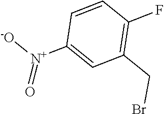

b. MB-22-Y(core): 5-[(2H-1,2,3,4-tetrazol-5-yl)methyl]-2H-1,2,3,4-tetrazole is commercially available

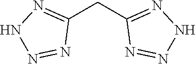 [SMILES: C(c1nn[nH]n1)c1nn[nH]n1]

c. MB-22-R-right synthesis:

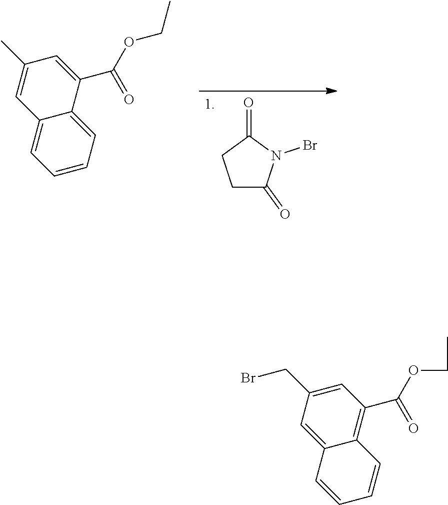 [SMILES: CCOC(=O)c1cc(C)cc2ccccc12 ; O=C1CCC(=O)N1Br ; CCOC(=O)c1cc(CBr)cc2ccccc12]

Ethyl (3-methyl-naphthalene-1-carboxylate) is heated with 1 equivalent of NBS and AIBN to form MB-22-R-right=ethyl (3-bromethyl-naphthalene-1-carboxylate) using the procedure in Liu, Wei-Min et al., Helvetica Chimica Acta, 2012, vol. 95 ,pp. 1953-1969.

MB-22 final assembly:

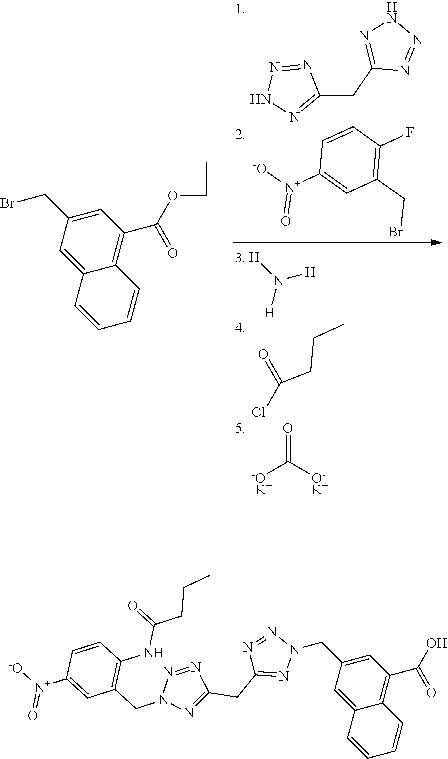

1. Ethyl (3-bromethyl-naphthalene-1-carboxylate) (1 equivalent) is added dropwise to 5-[(2H-1,2,3,4-tetrazol-5-yl)methyl]-2H-1,2,3,4-tetrazole and ethyl 3-({5-[(2H-1,2,3,4-tetrazol-5-yl)methyl]-2H-1,2,3,4-tetrazol-2-yl}methyl)naphthalene-1-carboxylate using General Procedure E (see Example 31).

2. ethyl 3-({5-[(2H-1,2,3,4-tetrazol-5-yl)methyl]-2H-1,2,3,4-tetrazol-2-yl}methyl)naphthalene-1-carboxylate and 2-(bromomethyl)-1-fluoro-4-nitrobenzene are combined to form ethyl 3-{[5-({2-[(2-fluoro-5-nitrophenyl)methyl]-2H-1,2,3,4-tetrazol-5-yl}methyl)-2H-1,2,3,4-tetrazol-2-yl]methyl}naphthalene-1-carboxylate using General Procedure E (see Example 31).

3. Ethyl 3-{[5-({2-[(2-fluoro-5-nitrophenyl)methyl]-2H-1,2,3,4-tetrazol-5-yl}methyl)-2H-1,2,3,4-tetrazol-2-yl]methyl}naphthalene-1-carboxylate is combined with excess ammonia in ethanol to form ethyl 3-{[5-({2-[(2-amino-5-nitrophenyl)methyl]-2H-1,2,3,4-tetrazol-5-yl}methyl)-2H-1,2,3,4-tetrazol-2-yl] methyl}naphthalene-1-carboxylate.

4. Ethyl 3-{[5-({2-[(2-amino-5-nitrophenyl)methyl]-2H-1,2,3,4-tetrazol-5-yl}methyl)-2H-1,2,3,4-tetrazol-2-yl]methyl}naphthalene-1-carboxylate is combined with butanoyl chloride under basic conditions to form ethyl 3-{[5-({2-[(2-butanamido-5-nitrophenyl)methyl]-2H-1,2,3,4-tetrazol-5-yl}methyl)-2H-1,2,3,4-tetrazol-2-yl]methyl}naphthalene-1-carboxylate.

5. Ethyl 3-{[5-({2-[(2-butanamido-5-nitrophenyl)methyl]-2H-1,2,3,4-tetrazol-5-yl}methyl)-2H-1,2,3,4-tetrazol-2-yl]methyl}naphthalene-1-carboxylate is converted to 3-{[5-({2-[(2-butanamido-5-nitrophenyl)methyl]-2H-1,2,3,4-tetrazol-5-yl}methyl)-2H-1,2,3,4-tetrazol-2-yl]methyl}naphthalene-1-carboxylic acid using potassium carbonate in refluxing ethanol.

### Example 36—Synthesis MB-23

### 5-{4-[2-(1-{2-hydroxy-5-[methoxy(methyl)carbamoyl]phenyl}-1H-1,2,3-triazol-4-yl)phenyl]-1H-1,2,3-triazol-1-yl}naphthalene-1-carboxylic acid

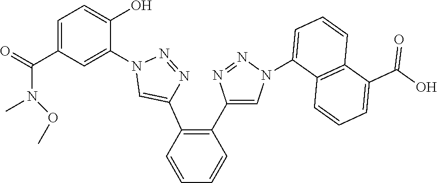 [SMILES: CON(C)C(=O)c1ccc(O)c(-n2cc(-c3ccccc3-c3cn(-c4cccc5c(C(=O)O)cccc45)nn3)nn2)c1]

a. MB-19-R′-left-3-azido-4-hydroxy-N-methoxy-N-methylbenzamide see Example 33 part a for synthesis.

b. MB-23-Y(core): 1,2-diethynylbenzene is commercially available.

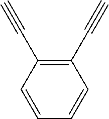

c. MB-19-R-right see example 33 part c for synthesis.

d. MB-23 final assembly:

The synthetic procedure in example 33, part d is used, but the precursors for MB-23 are MB-19-R′-left, MB-23-Y(core), and MB-19-R-right.

### Example 37—Synthesis of MB-24

### 5-{4-[2-(1-{2-acetamido-5-[methoxy(methyl)carbamoy]phenyl}-1H-1,2,3-triazol-4-yl)propan-2-yl]-1H-1,2,3-triazol-1-yl}naphthalene-1-carboxylic acid

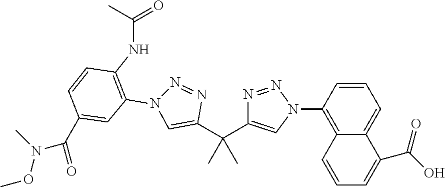 [SMILES: CON(C)C(=O)c1ccc(NC(C)=O)c(-n2cc(C(C)(C)c3cn(-c4cccc5c(C(=O)O)cccc45)nn3)nn2)c1]

### MB-24-R′-left: synthesis of 3-azido-4-acetamido-N-methoxy-N-methylbenzamide

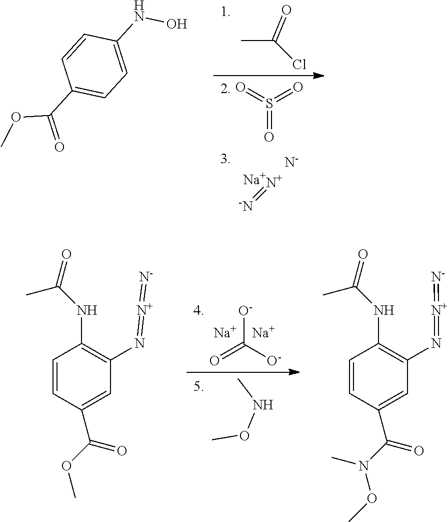

1. Synthesis of methyl 4-(N-hydroxyacetamido)benzoate. To a stirred suspension of methyl 4-(hydroxyamino) benzoate (0.900 g, 5.38 ml, 1.00 equiv) and NaHCO₃ (0.540 g, 6.46 mmol, 1.20 equiv) in Et₂0 (30.0 mL, 0.179 M) at 0° C. under N₂ is slowly added a solution of acetyl chloride (0.510 g, 6.46 mmol, 1.20 equiv) in Et₂O (30.0 mL, 0.215 M) via a syringe pump (at a rate of 10.0 mL/h). After the addition is complete, the reaction mixture is filtered through a short pad of celite and the celite is washed with EtOAc. The organic layers is combined and concentrated in vacuo. The residue is purified by chromatography on silica gel, eluting with hexanes: EtOAc (4:1 to 1:1 (v/v)), to afford the title compound as a light-yellow solid.

2. Synthesis of methyl 4-(N-hydroxyacetamido)benzoate-O-sulfate Methyl 4-(N-hydroxyacetamido)benzoate is combined with sulfur trioxide in pyridine/dichloromethane for 120 h at ambient temperature to form methyl 4-(N-hydroxyacetamido)benzoate-O-sulfate.

3. Synthesis of methyl 3-azido-4-acetamidobenzoate. 3-azido-4-acetamido-N-methoxy-N-methylbenzamide-O-sulfate is combined with sodium azide and tris-(2-chloro-ethyl)-amine in water/acetonitrile at 20° C. to form methyl 3-azido-4-acetamidobenzoate. This is purified on silica gel using ethyl acetate/hexane or methylene chloride/methanol.

4. Synthesis of 3. 3-azido-4-acetamido-N-methoxy-N-methylbenzamide (MB-25-R′-left) Methyl 3-azido-4-acetamidobenzoate is refluxed in ethanol with potassium carbonate until conversion to 3-azido-4-acetamidobenzoic acid is complete by TLC. After acidification to pH 3 with ethanolic HCl and filtration, the solvent is removed in vacuo to yield the crude 3-azido-4-acetamidobenzoic acid, which is used for the final step without further purification.

5. Synthesis of 3-azido-4-acetamido-N-methoxy-N-methylbenzamide 3-Azido-4-acetamidobenzoic acid is dissolved in an aprotic solvent (such as ethyl acetate, chloroform, or a mixture thereof) and added to a solution in the same solvent containing 1 equivalent of 1-Ethyl-3-(3-dimethylaminopropyl)carbodiimide (EDC), 0.1 equivalent DMAP, 1 equivalent of methylmethoxyamine hydrochloride, and 2 equivalents of diisopropylethylamine (DIEA). The reaction mixture is stirred at ambient temperature until conversion of 3-Azido-4-acetamidobenzoic acid to 3-azido-4-acetamido-N-methoxy-N-methylbenzamide is complete by TLC. The reaction mixture is acidified with HCl, stripped of solvent, and purified by silica gel chromatography using hexane/ethyl acetate or methylene chloride/methanol as the eluant, and stripped of solvent in vacuo to yield the title product.

b. MB-24-Y(core)-3,3-dimethyl-penta1,4-diyne

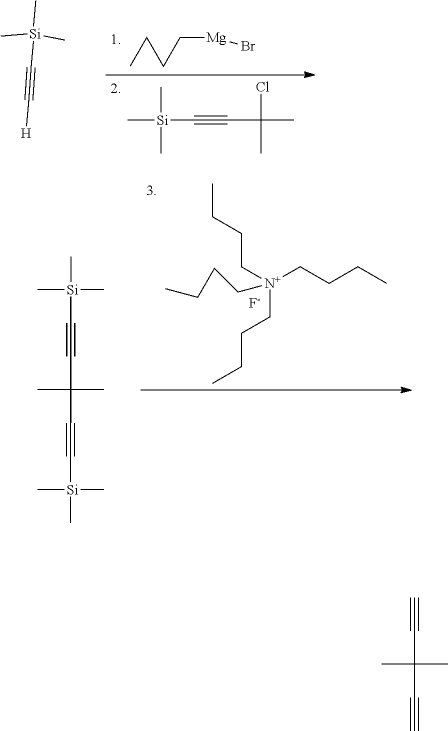

Trimethylsilylmagnesium bromide is prepared from a mixture of 1.28 ml (9 mmol) of trimethylsilylacetylene and 4.95 ml (9.9 mmol) of a 2M solution of butyl magnesium bromide in THF under nitrogen or argon at 0° C. One equivalent of (3-chloro-3-methylbut-1-yn-1-yl)trimethylsilane is then added as an ether solution. The reaction is stirred at 0° C. and followed by TLC or GC until the reaction is complete. The [3,3-dimethyl-5-(trimethylsilyl)penta-1,4-diyn-1-yl]trimethylsilane is purified by silica gel chromatography. [3,3-dimethyl-5-(trimethylsilyl)penta-1,4-diyn-1-yl]trimethylsilane is converted to MB-24-Y(core)=3,3-dimethyl-penta1,4-diyne using TBAF.

c. MB-19-R-right see example 33 part c for synthesis.

d. MB-24 final assembly: The synthetic procedure in MB-19 final assembly steps 1,3,5 is used, but the precursors for MB-24 are MB-24-R-left, MB-24-Y(core), and MB-19-R-right.

### Example 38—Synthesis of MB-25

### 5-[4-(4-{1-[2-acetamido-5-(methoxycarbonyl)phenyl]-1H-1,2,3-triazol-4-yl}phenyl)-1H-1,2,3-triazol-1-yl]naphthalene-1-carboxylic acid

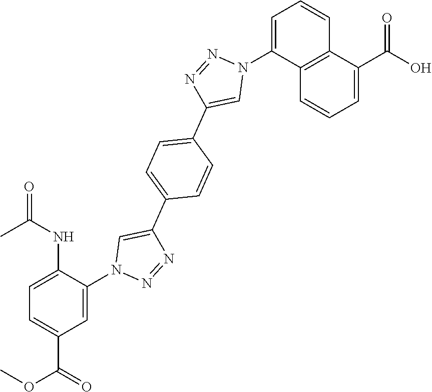 [SMILES: COC(=O)c1ccc(NC(C)=O)c(-n2cc(-c3ccc(-c4cn(-c5cccc6c(C(=O)O)cccc56)nn4)cc3)nn2)c1]

a. MB-25-R-left synthesis of methyl 3-azido-4-acetamidobenzoate: See example 24 part a, steps 1-3 for the synthetic procedure.

b. MB-25-Y(core)=1,4-diethynylbenzene is commercially available

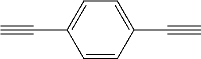

c. MB-19-R(right): see example 33 part c.

d. MB-25 final assembly:

The synthetic procedure in example 33 part d is used, but the precursors for MB-25 are MB-25-R′-left, MB-25-Y(core), and MB-19-R-right.

### Example 39—Synthesis of MB-26

### 5-[4-(3-{1-[2-acetamido-5-(methoxycarbonyl)phenyl]-1H-1,2,3-triazol-4-yl}phenyl)-1H-1,2,3-triazol-1-yl]naphthalene-1-carboxylic acid

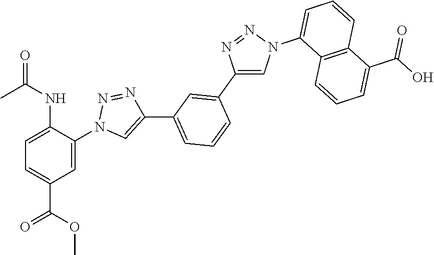 [SMILES: COC(=O)c1ccc(NC(C)=O)c(-n2cc(-c3cccc(-c4cn(-c5cccc6c(C(=O)O)cccc56)nn4)c3)nn2)c1]

a. MB-25-R′-left synthesis of methyl 3-azido-4-acetamidobenzoate see Example 38 part a.

b. MB-26-Y(core)=1,3-diethynylbenzene is commercially available.

c. MB-19-R-right: see example 33 part c for synthesis.

d. MB-26 final assembly:

The synthetic procedure in example 33d is used, but the precursors for MB-26 are MB-25-R-left, MB-26-Y(core), and MB-19-R-right.

### Example 40—Synthesis MB-27

### 5-[(4-{4-[1-({3-[methoxy(methyl)carbamoyl]phenyl}methyl)-1H-1,2,3-triazol-4-yl]phenyl}-1H-1,2,3-triazol-1-yl)methyl]naphthalene-1-carboxylic acid

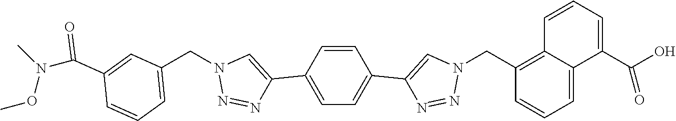 [SMILES: CON(C)C(=O)c1cccc(Cn2cc(-c3ccc(-c4cn(Cc5cccc6c(C(=O)O)cccc56)nn4)cc3)nn2)c1]

a. General Procedure C: Specific example: synthesis of MB-27-R′-left=3-(azidomethyl)-N-methoxy-N-methylbenzamide:

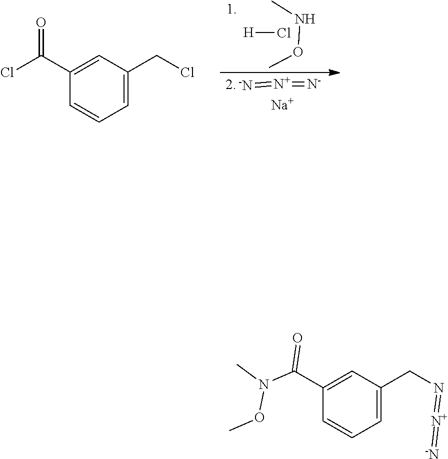

1. 3-(chloromethyl)-benzoyl chloride is combined with methyl methoxyamine hydrochloride in pyridine at 0° C. until conversion to 3-(chloromethyl)-N-methoxy-N-methylbenzamide is complete by TLC.

2. A solution of 1.2 equivalents of sodium azide in DMF is added to the reaction mixture and stirred at ambient temperature until formation of 3-(azidomethyl)-N-methoxy-N-methylbenzamide is complete by TLC. The reaction mixture is stripped of solvent in vacuo and purified by silica gel chromatography using hexane/ethyl acetate or methylene chloride/methanol as the eluant. The solvent is removed in vacuo, yielding the title product.

b. MB-25-Y-core—see example 38 for synthesis.

c. MB-27-Right Synthesis:

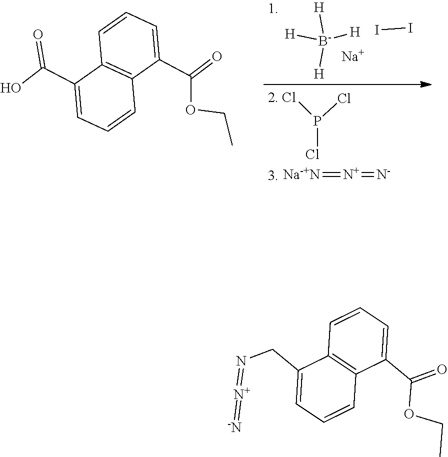

1. 5-(Ethoxycarbonyl)naphthalene-1-carboxylic acid is treated with sodium borohydride and iodine to form ethyl 5-(hydroxymethyl)naphthalene-1-carboxylate. The solvent is removed and the ethyl 5-(hydroxymethyl)naphthalene-1-carboxylate is purified by silica gel chromatography using hexane/ethyl acetate as the solvent.

2. Ethyl 5-(hydroxymethyl)naphthalene-1-carboxylate is treated with phosphorus trichloride to form ethyl 5-(chloromethyl)naphthalene-1-carboxylate. The reaction mixture is quenched with sodium bicarbonate in water, and the product is extracted with ether. The combined organic layers are dried over Na₂SO₄ and filtered. The solvent is removed from the filtrate and the ethyl 5-(chloromethyl) naphthalene-1-carboxylate is purified by silica gel chromatography using hexane/ethyl acetate as the solvent.

3. Ethyl 5-(chloromethyl) naphthalene-1-carboxylate is combined with sodium azide using General Procedure C (see Example 40) to form MB-27-R-left=3-(azidomethyl)-N-methoxy-N-methylbenzamide.

d. MB-27 final assembly.

The synthetic procedure in example 33 part d is used, but the precursors for MB-27 are MB-27-R′-left, MB-25-Y(core), and MB-27-R-right.

### Example 41—Synthesis MB-34

### 5-[4-(4′-{1-[2-hydroxy-5-(methoxycarbonyl)phenyl]-1H-1,2,3-triazol-4-yl}-[1,1′-biphenyl]-4-yl)-1H-1,2,3-triazol-1-yl]naphthalene-1-carboxylic acid

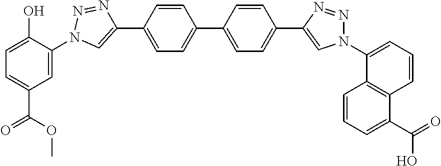 [SMILES: COC(=O)c1ccc(O)c(-n2cc(-c3ccc(-c4ccc(-c5cn(-c6cccc7c(C(=O)O)cccc67)nn5)cc4)cc3)nn2)c1]

a. MB-20-R′-left: see example 34 step a for synthesis.

b. MB-34-Y(core)=4,4′-diethynyl-biphenyl is commercially available.

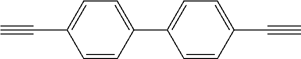 [SMILES: C#Cc1ccc(-c2ccc(C#C)cc2)cc1]

c. MB-19-R-right: see example 33 step c for synthesis.

d. MB-34 final assembly: The synthetic procedure in example 33, step d is used, but the precursors for MB-34 are MB-20-R′-left, MB-34-Y(core), and MB-19-R-right.

### Example 42—Synthesis of MB-35

### 5-[4-(6′-{1-[2-hydroxy-5-(methoxycarbonyl)phenyl]-1H-1,2,3-triazol-4-yl}-[3,3′-bipyridine]-6-yl)-1H-1,2,3-triazol-1-yl]naphthalene-1-carboxylic acid

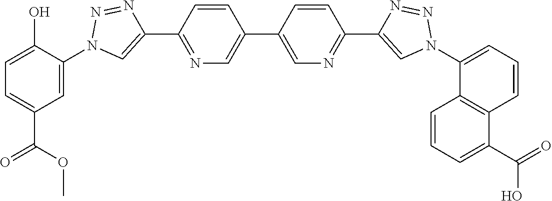 [SMILES: COC(=O)c1ccc(O)c(-n2cc(-c3ccc(-c4ccc(-c5cn(-c6cccc7c(C(=O)O)cccc67)nn5)nc4)cn3)nn2)c1]

a. MB-20-R′-left is synthesized as in example 34, step a.

b. MB-35-Y(core)=6,6′-diethynyl-3,3′-bipyridine

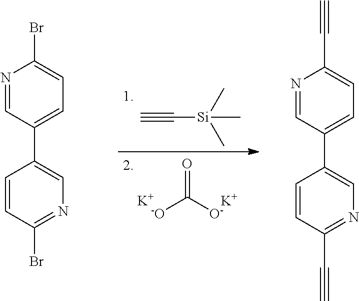

1. 6,6′-Dibromo-3,3′-bipyridine is combined with 2 equivalents of ethynyltrimethylsilane using General Procedure F (see Example 1) to form 6,6′-bis[2-(trimethylsilyl)ethynyl]-3,3′-bipyridine, which is purified using silica gel chromatography with hexane/ethyl acetate or methanol/methylene chloride.

2. 6,6′-bis[2-(trimethylsilyl)ethynyl]-3,3′-bipyridine is treated with K₂CO₃ in methanol/tetrahydrofuran to form title product MB-35-Y(core)=6,6′-diethynyl-3,3′-bipyridine, which is purified using silica gel chromatography with hexane/ethyl acetate or methanol/methylene chloride. Grosshenny, Vincent; Romero, Francisco M.; Ziessel, Raymond—[Journal of Organic Chemistry, 1997, vol. 62, # 5, p. 1491-1500]

c. MB-19-R-right see example 33, part c for synthesis.

d. MB-35 final assembly:

The synthetic procedure in example 33, part d is used, but the precursors for MB-35 are MB-20-R-left, MB-35-Y(core), and MB-19-R-right.

### Example 43—Synthesis of MB-37: N-{4-[4-(3-{1-[4-(acetamidosulfonyl)phenyl]-1H-1,2,3-triazol-4-yl}phenyl)-1H-1,2,3-triazol-1-yl]benzenesulfonyl}-N-pentylacetamide

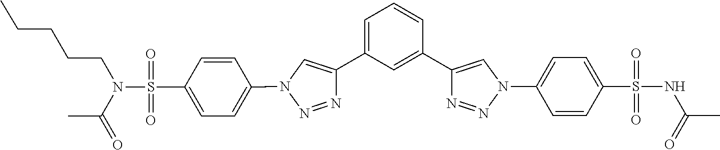 [SMILES: CCCCCN(C(C)=O)S(=O)(=O)c1ccc(-n2cc(-c3cccc(-c4cn(-c5ccc(S(=O)(=O)NC(C)=O)cc5)nn4)c3)nn2)cc1]

a. MB-37-R-left=N-(4-azidobenzene sulfonyl)acetamide synthesis (note that MB-37-R-right and MB-37-R′-left are identical).

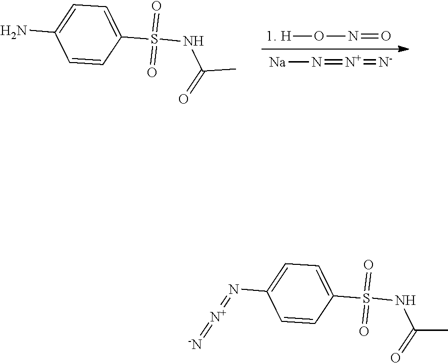

1. 4-amino-N-acetylbenzenesulfonamide (3.0 mmol) is added in a solution of concentrated sulfuric acid (0.5 mL) and H₂O (3.0 mL) and then cooled to 0 C in an ice bath. A solution of NaNO₂ (3.0 mmol) in water (2.1 mL) is added dropwise to the reaction and left stirring for 10 min. After a color change to a yellowish tone and appearance of foam in the medium, solid urea (150 mg) is added followed by NaN₃ solution (9.0 mmol, 1.5 eq) in H₂O (3.2 mL) dropwise. Finally, after stirring for few minutes each reaction is filtered through Buchner funnel and washed subsequently with 5 percent NaHCO₃ and H₂O. Product is dried under reduced pressure. Product is optionally purified by recrystallization from water/ethanol or isopropanol.

b. MB-37-Y-core=1,3-diethynylbenzene is commercially available.

c. MB-37-R-right: see MB-37-R′-left (identical molecules)

d. MB-37=N-{4-[4-(3-{1-[4-(acetamido sulfonyl)phenyl]-1H-1,2,3-triazol-4-yl}phenyl)-1H-1,2,3-triazol-1-yl]benzenesulfonyl}-N-pentylacetamide

Final Assembly:

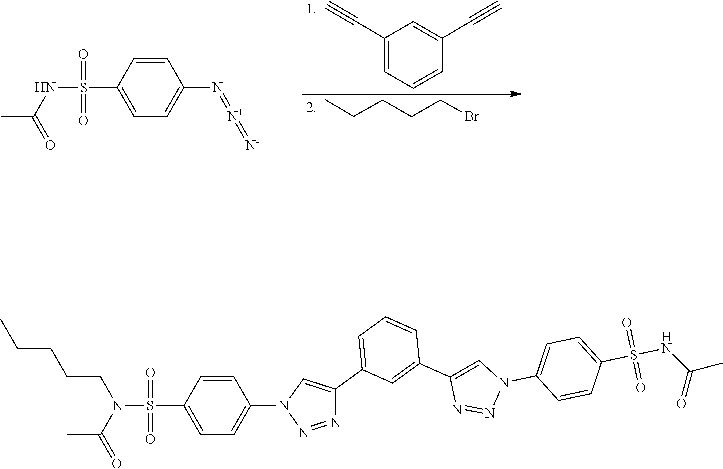

1. MB-37-Y-core=1,3-diethynylbenzene is combined with 2 equivalents of MB-37-R′-left=MB-37-R-right=N-(4-azidobenzene sulfonyl)acetamide using General Procedure U (see Example 6) to form N-{4-[4-(3-{1-[4-(acetamidosulfonyl)phenyl]-1H-1,2,3-triazol-4-yl}phenyl)-1H-1,2,3-triazol-1-yl]benzenesulfonyl}-acetamide

2. 1 equivalent of 1-bromopentane is added dropwise to a solution of N-{4-[4-(3-{1-[4-(acetamidosulfonyl)phenyl]-1H-1,2,3-triazol-4-yl}phenyl)-1H-1,2,3-triazol-1-yl]benzenesulfonyl}-acetamide and 1 equivalent of DIPEA. The reaction is followed by TLC until the monoalkylated product N-{4-[4-(3-{1-[4-(acetamidosulfonyl) phenyl]-1H-1,2,3-triazol-4-yl}phenyl)-1H-1,2,3-triazol-1-yl]benzenesulfonyl}-N-pentylacetamide has reached a maximum. The title product is purified using silica gel chromatography with methylene chloride/methanol. Note that there is no deprotection step needed for MB-37.

### Example 51—Synthesis of MB-45

3-[(5-{[2({3-[methoxy(methyl)carbamoyl]phenyl}methyl)-2H-1,2,3,4-tetrazol-5-yl]methyl}-2H-1,2,3,4-tetrazol-2-yl)methyl]benzoic acid is made entirely from commercially available precursors in 4 steps.

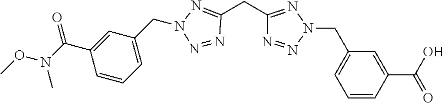 [SMILES: CON(C)C(=O)c1cccc(Cn2nnc(Cc3nnn(Cc4cccc(C(=O)O)c4)n3)n2)c1]

a. MB-45-R′-left=MB-45-R-right=tert-butyl 3-(bromomethyl)benzoate is commercially available.

b. MB-22-Y(core)=5-[(2H-1,2,3,4-tetrazol-5-yl)methyl]-2H-1,2,3,4-tetrazole is commercially available.

c. MB-45-R-right=MB-45-R′-left=tert-butyl 3-(bromomethyl)benzoate is commercially available.

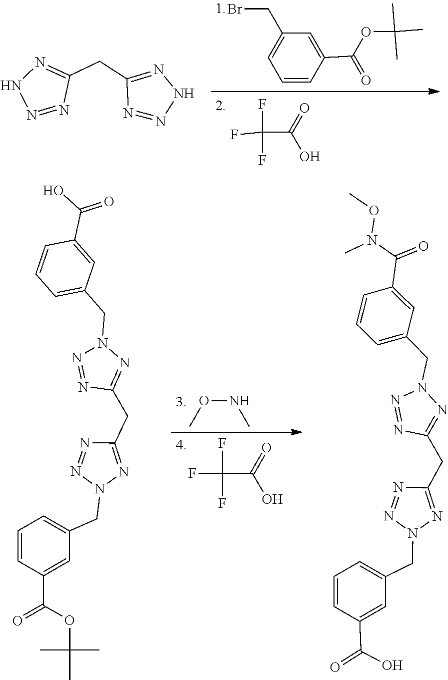

MB-45 final assembly:

1 Two equivalents of MB-45-R′-left=MB-45-R-right=tert-butyl 3-(bromomethyl)benzoate are added dropwise to MB-22-Y(core)=5-[(2H-1,2,3,4-tetrazol-5-yl)methyl]-2H-1,2 ,3 ,4-tetrazole to form tert-butyl 3-[(5-{[2-({3-[(tert-butoxy)carbonyl]phenyl}methyl)-2H-1,2,3,4-tetrazol-5-yl]methyl}-2H-1,2,3,4-tetrazol-2-yl)methyl]benzoate using General procedure E (see Example 31). The product is purified using silica gel chromatography.

2. 1 equivalent of TFA is added dropwise to diester Tert-butyl 3-[(5-{[2-({3-[(tert-butoxy) carbonyl] phenyl}methyl)-2H-1,2,3,4-tetrazol-5-yl] methyl}-2H-1,2,3,4-tetrazol-2-yl)methyl]benzoate using General Procedure X (see Example 12). to generate the monoacid/ester 3-[(5-{[2-({3-[(tert-butoxy) carbonyl] phenyl}methyl)-2H-1,2,3,4-tetrazol-5-yl] methyl}-2H-1,2,3,4-tetrazol-2-yl)methyl]benzoic acid.

3. 3-[(5-{[2-({3-[(tert-butoxy) carbonyl] phenyl}methyl)-2H-1,2,3,4-tetrazol-5-yl] methyl}-2H-1,2,3,4-tetrazol-2-yl)methyl]benzoic acid is combined with N-methyl-N-methoxyamine to form tert-butyl 3-[(5-{[2-({3-[methoxy(methyl)carbamoyl]phenyl} methyl)-2H-1,2,3,4-tetrazol-5-yl]methyl}-2H-1,2,3,4-tetrazol-2-yl)methyl]benzoate using General Procedure Y (see Example 16).

4. Tert-butyl 3-[(5-{[2-({3-[methoxy(methyl)carbamoyl]phenyl} methyl)-2H-1,2,3,4-tetrazol-5-yl]methyl}-2H-1,2,3,4-tetrazol-2-yl)methyl]benzoate is converted to MB-35=3-[(5-{[2-({3-[methoxy(methyl)carbamoyl]phenyl}methyl)-2H-1,2,3,4-tetrazol-5-yl]methyl}-2H-1,2,3,4-tetrazol-2-yl)methyl]benzoic acid using General Procedure X (see Example 12).

### Example 44—Synthesis of MB-47

### 5-[(6-chloro-3-{[(5-methanesulfonyl-1H-1,3-benzodiazol-2-yl)methyl]amino}-1,2,4-triazin-5-yl)carbamoyl]naphthalene-1-carboxylic acid

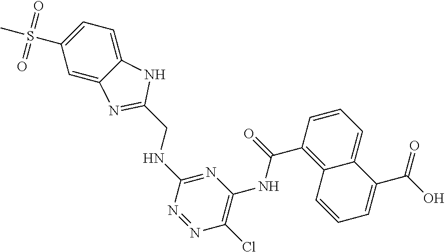 [SMILES: CS(=O)(=O)c1ccc2[nH]c(CNc3nnc(Cl)c(NC(=O)c4cccc5c(C(=O)O)cccc45)n3)nc2c1]

a. MB-47-R′-left synthesis: Synthesis of (5-methanesulfonyl-1H-1,3-benzodiazol-2-yl)methanamine:

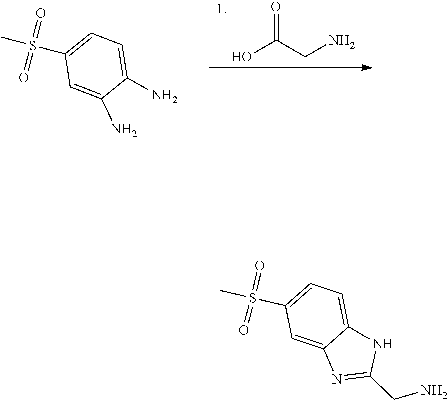

4-Methanesulfonylbenzene-1,2-diamine is combined with glycine to yield (5-methanesulfonyl-1H-1,3-benzodiazol-2-yl)methanamine, using a procedure similar to Elshihawy, Hosam; Helal, Mohamed A.; Said, Mohamed; Hammad, Mohamed A., Bioorganic and Medicinal Chemistry, 2014, vol. 22, #1, p. 550-558.

b. MB-47-Y(core)=5-amino-3,6-dichloro-1,2,4-triazine is commercially available.

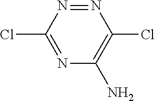

c. MB-47-R-right=5-[(tert-butoxy)carbonyl]naphthalene-1-carboxylic acid.

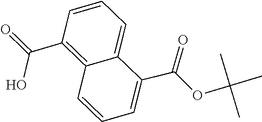 [SMILES: CC(C)(C)OC(=O)c1cccc2c(C(=O)O)cccc12]

1. 5-Bromonapthalene-1-carboxylic acid is converted to t-butyl 5-bromonapthalene-1-carboxylate using General Procedure G (see Example 1)

2. T-butyl 5-bromonapthalene-1-carboxylate is treated with n-butylithium in anhydrous THF/hexane under argon or nitrogen at −90° C. for 30 minutes, then with carbon dioxide at −90° C. for 10 minutes to form MB-47-R-right=5-[(test-butoxylcarbonyl] naphthalene-1-carboxylic acid.

d. MB-47 final assembly:

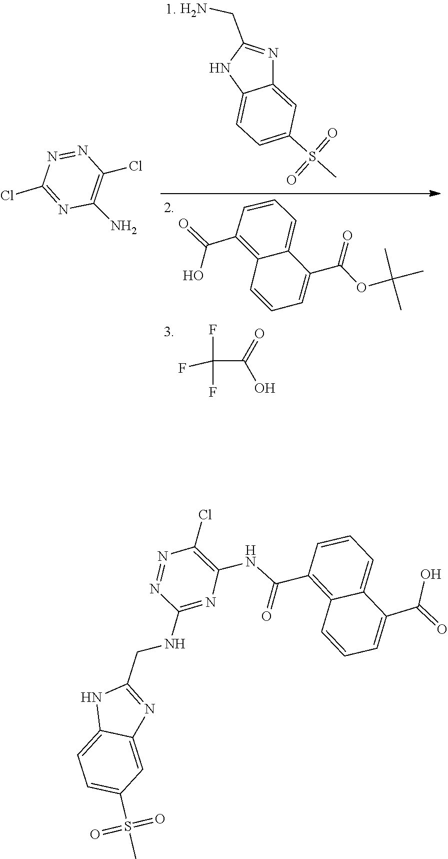

1. MB-47-R′-left=(5-methanesulfonyl-1H-1 ,3-benzodiazol-2-yl)methanamine preferentially displaces the more reactive 3-chlorine in MB-47-Y(core)=5-amino-3,6-dichloro-1,2,4-triazine in the presence of 1 equivalent of DIPEA to form 6-chloro-N3-[(5-methanesulfonyl-1H-1,3-benzodiazol-2-yl)methyl]-1,2,4-triazine-3,5-diamine.

2. 6-chloro-N3-[(5-methanesulfonyl-1H-1,3-benzodiazol-2-yl)methyl]-1,2,4-triazine-3,5-diamine is combined with 5-[(tert-butoxy)carbonyl]naphthalene-1-carboxylic acid to form t-butyl 5-[(6-chloro-3-{[(5-methanesulfonyl-1H-1,3-benzodiazol-2-yl)methyl] amino}-1,2,4-triazin-5-yl)carbamoyl]naphthalene-1-carboxylate using General Procedure I.

3. T-butyl 5-[(6-chloro-3-{[(5-methanesulfonyl-1H-1,3-benzodiazol-2-yl)methyl] amino}-1,2,4-triazin-5-yl)carbamoyl]naphthalene-1-carboxylate is converted to MB-47=5-[(6-chloro-3-{[(5-methanesulfonyl-1H-1,3-benzodiazol-2-yl)methyl]amino}-1,2,4-triazin-5-yl)carbamoyl]naphthalene-1-carboxylic acid using General Procedure X (see Example 12).

### Example 45—Synthesis of MB-48

### 2-[5-amino-3-(4-{4-[methoxy(methyl)carbamoyl]benzamido}-4-methylpiperidin-1-yl)-1,2,4-triazin-6-yl]pyridine-4-carboxylic acid

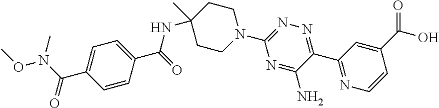 [SMILES: CON(C)C(=O)c1ccc(C(=O)NC2(C)CCN(c3nnc(-c4cc(C(=O)O)ccn4)c(N)n3)CC2)cc1]

a. MB-48-R′-left=4-(N-methoxy-N-methyl-carbamoyl)-benzoic acid is commercially available (CAS #1431880-66-8).

b. MB-48-Y(core)=tert-butyl N-[1-(5-amino-6-bromo-1,2,4-triazin-3-yl)-4-methylpiperidin-4-yl]carbamate is commercially available: AstaTech (#92271).

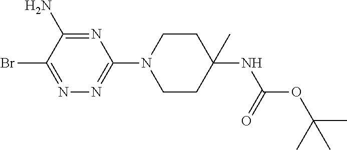 [SMILES: CC1(NC(=O)OC(C)(C)C)CCN(c2nnc(Br)c(N)n2)CC1]

c. MB-48-R-right=methyl 2-(4,4,5,5-tetramethyl-1,3,2-dioxaborolan-2-yl)pyridine-4-carboxylate is commercially available from CombiPhos Catalysts (#BE409).

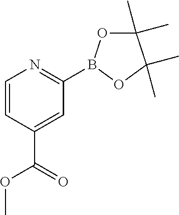 [SMILES: COC(=O)c1ccnc(B2OC(C)(C)C(C)(C)O2)c1]

d. MB-48 final assembly:

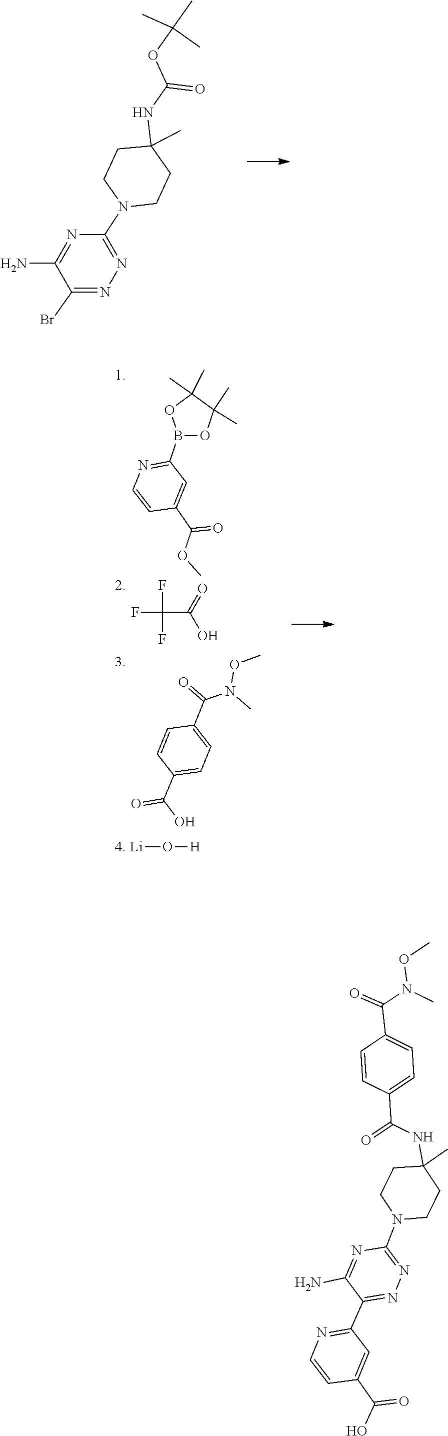

1. MB-48-R′-left=4-(N-methoxy-N-methyl-carbamoyl)-benzoic acid is combined with MB-48-Y(core)=tert-butyl N-[1-(5-amino-6-bromo-1,2,4-triazin-3-yl)-4-methylpiperidin-4-yl]carbamate are coupled to form Tert-butyl 2-[5-amino-3-(4-amino-4-methylpiperidin-1-yl)-1,2,4-triazin-6-yl]pyridine-4-carboxylate using General Procedure Z part a (See Example 2).

2. Tert-butyl 2-[5-amino-3-(4-amino-4-methylpiperidin-1-yl)-1,2,4-triazin-6-yl]pyridine-4-carboxylate is converted to 2-[5-amino-3-(4-amino-4-methylpiperidin-1-yl)-1,2,4-triazin-6-yl]pyridine-4-carboxylic acid using General Procedure X (see Example 12).

3. 2-[5-Amino-3-(4-amino-4-methylpiperidin-1-yl)-1,2,4-triazin-6-yl]pyridine-4-carboxylic acid is combined with 4-(N-methoxy-N-methyl-carbamoyl)-benzoic acid to form Methyl 2-[5-amino-3-(4-{4-[methoxy (methyl)carbamoyl] benzamido}-4-methylpiperidin-1-yl)-1,2,4-triazin-6-yl]pyridine-4-carboxylate using General Procedure X (see Example 12).

4. Methyl 2-[5-amino-3-(4-{4-[methoxy (methyl)carbamoyl]benzamido}-4-methylpiperidin-1-yl)-1,2,4-triazin-6-yl]pyridine-4-carboxylate is converted to MB-48=2-[5-amino-3-(4-{4-[methoxy(methyl)carbamoyl]benzamido}-4-methylpiperidin-1-yl)-1,2,4-triazin-6-yl]pyridine-4-carboxylic acid using General Procedure O (see Example 24).

### REFERENCES FORMULA II COMPOUNDS

NOVARTIS AG; BEATTIE, David; et al.,—International Publication No. WO2015/8230, column 320; 321
ELI LILLY AND COMPANY—International Publication No WO2004/14900, Page 24; 60
NOVARTIS AG; BEATTIE, David; et al.,—International Publication No WO2015/8230
THE RESEARCH FOUNDATION FOR THE STATE UNIVERSITY OF NEW YORK; NGAI, Ming-Yu; HOJCZYK, Katarzyna, N.—International Publication No WO2016/57931
Novak, et al.—JACS V 106 pp. 5623-5631, 1984
Novak, et al., Journal of Organic Chemistry, p. 8294-8304, 1995.
Amgen Inc.—US Publication No. 2006/199796, A1 column 37; 38
MEMORY PHARMACEUTICALS CORPORATION—International Publication No WO2006/44528
CS PHARMASCIENCES, INC.; SONG, Yuntao; BRDIGES, Alexander, James—International Publication No WO2017/120429
MB-35-Y(core): Grosshenny, Vincent; Romero, Francisco M.; Ziessel, Raymond—Journal of Organic Chemistry, 1997, vol. 62, #5, p. 1491-1500]
Brown, Thomas B.; Lowe, Philip R.; Schwalbe, Carl H.; Stevens, Malcolm F. G.—[Journal of the Chemical Society, Perkin Transactions 1: Organic and Bio-Organic Chemistry (1972-1999), 1983, #10, p. 2485-2490]
Baldwin, Jack E.; Schofield, Christopher J.; Smith, Bradley D.—Tetrahedron, 1990, vol. 46, #8, p. 3019 3028.

### Example 46—Activity of Compounds

The activity of the disclosed compounds to act as inhibitors of receptor binding and function of TNF family cytokine CD40 are tested as described in Bojadzic, D. et al. (2018), Molecules, 23:1153; Chen, J. et al. (2017), J. Med. Chem, 60(21): 8906-8922; Silvian, L. (2011) ACS Chem Biol., 6(6): 636-647; and Aarts S., et al., (2017) Journal of Neuroinflammation, 14:105.

All references cited in the present application are incorporated herein by reference.

A number of embodiments of the disclosure have been described. Nevertheless, it will be understood that various modifications may be made without departing from the spirit and scope of the disclosure. Accordingly, other embodiments are within the scope of the following claims.

What is claimed is:

1. A compound of Formula I: 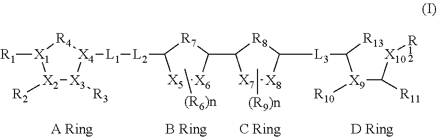
or a pharmaceutically acceptable salt, ester, prodrug, hydrate and tautomer thereof, wherein:
Ring A is an optionally substituted 6-membered or 5-membered aryl, heteroaryl, cycloalkyl, cycloalkenyl, or heterocycloalkyl ring;
X₁, X₂, X₃, X₄ are each separately and independently selected from the group consisting of C, or N;
R₄ is selected from the group consisting of CH═CH, CH, S, O, N, N═CH, CH═N, N═N, and CH₂CH₂, with the proviso that if any of X₁, X₂, X₃, X₄ are N, then R₄ is restricted to CH;
R₁ is selected from the group consisting of SO₂NR′₂, SO₂R′, COR′, COOR′, CON(R′)₂, CON(OR′)R′, tetrazole, triazole, optionally linked to the A ring through a bond, C₁-C₃ alkyl chain, a 6-membered or 5-membered aryl, a 5 or 6-membered cycloalkyl, a 5- or 6-membered heterocycloalkyl, or 6-membered or membered heteroaryl; wherein each R′ is independently C₁-C₆ alkyl, C₂-C₆ heteroalkyl, 2-methoxyethyl, 2′-(2-methoxyethoxy)ethyl wherein each R′ can be optionally substituted with one or more groups selected from fluorine, C₁-C₄alkyl, C₁-C₄heteroalkyl, and ═O;
R₂ is selected from the group consisting of H, optionally substituted C₁-C₃ alkylSO₂R′, SO₂NR′₂, COOR′, CON(R′)₂, CON(OR)R′, C₁-C₆ alkenyl, C₁-C₆ alkynyl, tetrazole, triazole, optionally linked to the A ring through a bond, wherein each R′ is independently H, C₁-C₆ alkyl, C₃-C₉cycloalkyl-alkyl, or C₂-C₁₃ heteroalkyl (in which 1 to 4 carbons are replaced with oxygen), wherein R′ can be optionally substituted with one or more groups selected from fluorine, CH₃; or
R₁ and R₂ taken together, form a fused ring with ring A to form benztriazole, substituted on either the 1- or 2-nitrogen with C₁-C₃ alkyl, 2-methoxyethyl, 2-(2′-methoxyethoxy-ethyl), (CH₂)_{W}COOR′, (CH₂)_{W}CON(OR′)R′, wherein R′ is C₁-C₃ alkyl and w is 0-3;
L₁ is a single bond, absent, —NHCO—, CONH—, 1,3,4-thiadiazole-2,5-diyl or else forms part of a ring with R₃;
L₂ is absent or a single bond or is thia-3,4-thiadiazole-2,5-diyl, —CONH—, —NHCO—, CONHCH₂—, —NH—, —NHCH(CF₃)—, CON(CH₃)SO₂—, SO₂N(CH₃)CO, —CCF₃—NH—, —SOCH₂—, or —S(O)(NR₁₈)NH—, wherein R₁₈ is selected from C₁-C₆ alkyl, C₄-C₁₀ oxa-alkyl, C₄-C₁₀ dioxa-alkyl, or C₄-C₁₀ trioxa-alkyl;
Ring B is an optionally substituted 6-membered or 5-membered aryl, or heteroaryl;
X₅ and X₆ of Ring B are independently and separately selected from the group consisting of C or N;
R₆ attached to X₆ of Ring B is separately and independently H, F, Cl, Br, I; or
if R₆ is attached to X₅, R₆ and L₁ taken together, along with the two intervening atoms to which they are attached, form an optionally substituted five-membered ring optionally substituted heterocycloalkyl ring having 2-3 heteroatoms independently selected from N, and S; wherein the rings are optionally substituted with one or more substituents selected from ═O, C₁-C₆ alkyl, or C₂-C₁₃ heteroalkyl, wherein the heteroatoms are 1 to 3 oxygen atoms);
R₇ is CH═CH, N═CH, O or S;
Ring C is an optionally substituted 6-membered or 5-membered aryl, or heteroaryl;
X₇ and X₈ of Ring C are independently and separately selected from the group consisting of C, or N;
L₃ is absent, a single bond, or selected from the group consisting of —CONH—, —NHCO—, CONHCH₂—, —NH—, —NHCH(CF₃)—, CON(CH₃)SO₂—, SO₂N(CH₃)CO—, —CH₂SO—, SOCH₂, —CH(CF₃)—NH— and —CONHSO₂—;
Ring D is an optionally substituted 6-membered aryl, or heteroaryl ring;
X₉ and X₁₀ of Ring D are independently and separately selected from the group consisting of C, or N;
R₁₃ of Ring D is —CH═N—, CH═CH, —N═C—, N═N, or S, all of which can be optionally substituted on carbon atoms except for S and N═N;
R₁₀, R₁₁, R₁₂ are independently and separately selected from H, F, C₁-C₆alkyl, CH₂COOH, CH(CH₃)COOH, COOH, SO₂NHCOR′, and CONHSO₂R′, wherein each R′ is independently C₁-C₆ alkyl, C₂-C₆ heteroalkyl, 2-methoxyethyl, 2′-(2-methoxyethoxy)ethyl wherein each R′ can be optionally substituted with one or more of fluorine, C₁-C₄alkyl, C₁-C₄ heteroalkyl, and ═O, wherein exactly one of R₁₀, R₁₁, or R₁₂ is CH₂COOH or COOH, provided R₁₀ and R₁₁ do not combine to form a 6-membered ring; or
R₁₀ and R₁₁ taken together, along with the two intervening atoms to which they are attached, form an optionally substituted five or six-membered aromatic, aliphatic heteroaromatic, or heteroaliphatic ring, so that ring D, R₁₀ and R₁₁ taken together form a bicyclic ring system, wherein the bicyclic ring system is substituted with exactly one substituent selected from COOH, SO₂NHCOR′, CONHSO₂R′, CH₂COOH, and CH(CH₃)COOH, wherein each R′ is independently C₁-C₆ alkyl, C₂-C₆ heteroalkyl, 2-methoxyethyl, or 2′-(2-methoxyethoxy)ethyl and wherein each R′ can be optionally substituted with one or more groups selected from fluorine, C₁-C₄alkyl, C₁-C₄ heteroalkyl, and ═O, and R₁₂ is H, F, or C₁-C₆ alkyl.

2. The compound of claim 1, wherein Ring A is optionally substituted phenyl, 1,3,4-thiadiazole, 1,2,3-triazole, 1,2,4-triazole, or piperidine.

3. The compound of claim 2, wherein Ring A is optionally substituted phenyl.

4. The compound of claim 2, wherein Ring A is optionally substituted 1,3,4-thiadiazole.

5. The compound of claim 2, wherein Ring A is optionally substituted piperidine.

6. The compound of claim 1, wherein Ring A is benzo[d]isothiazol-3(2H)-one 1,1-dioxide.

7. The compound of claim 1, wherein Ring A is 2,3-dihydro-1H-isoindole-1,3-dione.

8. The compound of claim 1, wherein, X₁, X₂, X₃, X₄ are all C.

9. The compound of claim 1, wherein X₁, X₂, X₃ are C and X₄ is N.

10. The compound of claim 1, wherein R₄ is substituted with a 5-membered heteroaryl ring.

11. The compound of claim 10, wherein the 5-membered heteroaryl ring is substituted with a 1,2,3-triazole or 1,2,4-triazole.

12. The compound of claim 10, wherein the 5-membered heteroaryl ring is substituted with a tetrazole.

13. The compound of claim 10, wherein R₁ is H.

14. The compound of claim 1, wherein R₁, is selected from the group of phenyl, triazole, tetrazole, and furan which can all be optionally substituted.

15. The compound of claim 14, wherein R₁ is triazole, tetrazole, or furan and is optionally substituted with a group selected from C₁-C₆ alkyl, C₁-C₆ alkenyl, C₁-C₆ alkynyl and COR′, wherein the alkyl, alkenyl or alkynyl can be substituted with a C₃-C₆ cycloalkyl.

16. The compound of claim 14, wherein R₁ is phenyl and is optionally substituted with a group selected from SO₂NR′₂, COR′, COOR′, CON(R′)₂, CON(OR′)R′, SO₂R′, tetrazole, or triazole, wherein each R′ is independently C₁-C₆ alkyl, C₂-C₆heteroalkyl, 2-methoxyethyl, 2′-(2-methoxyethoxy)ethyl wherein each R′ can be optionally substituted with one or more groups selected from fluorine, C₁-C₄alkyl, C₁-C₄ heteroalkyl, and ═O.

17. The compound of claim 17, wherein the phenyl is substituted with a group selected from COOCH₃, CON(CH₃)₂, 5-ethyl-2H-tetrazol and (3-(2-methoxyethoxy)prop-1-yn-1-yl.

18. The compound of claim 1, wherein R₁ is piperidine that is optional substituted with a group selected from SO₂NR′₂, COR′, COOR′, CON(R′)₂, CON(OR′)R′, SO₂R′, tetrazole, and triazole.

19. The compound of claim 18, wherein the piperidine is substituted with COOCH₃.

20. The compound of claim 14, wherein R₁ is furan that is optionally substituted with a group selected from SO₂NR′₂, COR′, COOR′, CON(R′)₂, CON(OR′)R′, SO₂R′, wherein each R′ is independently C₁-C₆ alkyl, C₂-C₆ heteroalkyl, 2-methoxyethyl, 2′-(2-methoxyethoxy)ethyl wherein each R′ can be optionally substituted with one or more groups selected from fluorine, C₁-C₄alkyl, C₁-C₄ heteroalkyl, and ═O.

21. The compound of claim 20, wherein the furan is substituted with COOCH₃.

22. The compound of claim 1, wherein R₂ is tetrazole or triazole that is optionally substituted with a group selected from C₁-C₆ alkyl or C₄-C₁₀ oxa-alkyl, dioxa-alkyl, or trioxa-alkyl.

23. The compound of claim 22, wherein the C₁-C₆ alkyl is optionally substituted with a 3-6 cyclo-alkyl at its terminal carbon.

24. The compound of claim 1 or 22, wherein R₂ is tetrazole that can be optionally substituted with the group selected from C₁-C₆ alkyl, C₃-C₉ cycloalkyl-alkyl, and C₂-C₁₃ oxaalkyl (in which 1 to 4 carbons are replaced with oxygen).

25. The compound of claim 1, wherein R₃ is 2-alkyl-ethynyl (C₁-C₄ alkyl) or (3-(2-methoxyethoxy)prop-1-yn-1-yl, wherein the C₁-C₄ alkyl is optionally substituted with C₃-C₆ cycloalkyl at the C-terminus, and the alkyl and cycloalkyl are optionally substituted on carbon with 1 or more fluorine atoms.

26. The compound of claim 1, wherein R₃ is absent or selected from H, F, and methyl.

27. The compound of claim 1, wherein R₁ and R₂ taken together, form a fused ring with ring A to form benztriazole, that is always substituted on either the 1- or 2-nitrogen with the group selected from C₁-C₃ alkyl, 2-methoxyethyl, 2-(2′-methoxyethoxy-ethyl), (CH₂)_{w}COOR′, and (CH₂)_{w}CON(OR′)R′, wherein R′ is C₁-C₃ alkyl and w is 0-3.

28. The compound of claim 1 or 27, wherein R₁ and R₂ taken together, form a fused ring with ring A to form benzo[d]isothiazol-3(2H)-one 1,1-dioxide or 2,3-dihydro-1H-isoindole-1,3-dione optionally substituted on nitrogen with the group selected from C₁-C₃ alkyl, 2-methoxyethyl, 2-(2′-methoxyethoxy-ethyl), (CH₂)_{w}COOR′, (CH₂)_{w}CON(OR′)R′, wherein R′ is C₁-C₃ alkyl and w is 0-3, wherein each R′ is independently C₁-C₆ alkyl, C₂-C₆ heteroalkyl, 2-methoxyethyl, 2′-(2-methoxyethoxy)ethyl wherein each R′ can be optionally substituted with one or more groups selected from fluorine, C₁-C₄alkyl, C₁-C₄ heteroalkyl, and ═O.

29. The compound of claim 1, wherein R₃ and L₁ taken together, along with the two intervening atoms to which they are attached, form an optionally substituted heterocycloalkyl ring having 1-3 heteroatoms independently selected from N, O, and S; wherein the rings are optionally substituted with one or more substituents selected from halo, C₁-C₃ alkyl, 2-methoxyethyl or 2-(2′-methoxyethoxy-ethyl).

30. The compound of claims 1-29, wherein Ring B is an optionally substituted phenyl, pyridazine, pyridine, or thiophene.

31. The compound of claim 30, wherein if R₆ is attached to X₅, R₆ and L₁ taken together, along with the two intervening atoms to which they are attached, form an optionally substituted five-membered optionally substituted heterocycloalkyl ring having 2-3 heteroatoms independently selected from N, and S; wherein the rings are optionally substituted with one or more substituents selected from ═O, C₁-C₆ alkyl, and C₂-C₁₃ heteroalkyl wherein the heteroatoms are 1 to 3 oxygen atoms.

32. The compound of claims 1-31, wherein R₇ is selected from CH═CH, CF═CH, N═CH, O or S.

33. The compound of claims 1-31, wherein Ring C is an optionally substituted 6-membered or 5-membered aryl, or heteroaryl

34. The compound of claim 34, wherein X₇ and X₈ of Ring C are independently and separately selected from the group consisting of C, or N.

35. The compound of claims 1-34, wherein Ring C is optionally substituted phenyl, pyridazine, pyridine, thiophene, or furan.

36. The compound of claims 1-35, wherein when R₉ is attached to X₇ it is H, or F.

37. The compound of claims 1-36, wherein L₃ is absent, a single bond, or selected from the group consisting of —CONH—, —NHCO—, CONHCH₂—, —NH—, —NHCH(CF₃)—, CON(CH₃)SO₂—, SO₂ N(CH₃)CO and —CCF₃—NH—.

38. The compound of claims 1-37, wherein when R₉ is attached to X₈, then R₉, X₈ and L₃ taken together, along with intervening atoms to which they are attached, can form an optionally substituted five-membered heteroaromatic or heterocycloalkyl ring having 2-3 heteroatoms independently selected from N, O, and S; wherein the rings are optionally substituted with one or more substituents selected from halo, ═O, H, C₁-C₆ alkyl, and C₂-C₁₁ heteroalkyl (with 1-3 oxygens).

39. The compound of claims 1-38, wherein R₈ is selected from CH═CH, CH═CF, C═N, S, and O.

40. The compounds of claims 1-39, wherein Ring D is an optionally substituted 6-membered aryl, or heteroaryl rings;

41. The compound of claim 40, wherein X₉ and X₁₀ are independently and separately selected from the group consisting of C, or N.

42. The compound of claim 40, wherein R₁₃ is —C═N—, CH═CH, —N═C—, N═N, or S, all of which can be optionally substituted on carbon atoms except for S and N═N.

43. The compound of claim 42, wherein R₁₀, R₁₁, R₁₂ are independently and separately selected from the group consisting of H, F, C₁-C₆ alkyl, CH₂COOH, CH(CH₃)COOH or COOH, where exactly one of R₁₀, R₁₁, or R₁₂ is CH₂COOH or COOH, provided R₁₀ and R₁₁ do not combine to form a 6-membered ring.

44. The compound of claim 1 or claim 42, wherein Ring D, R₁₀, and R₁₁ form a bicyclic ring selected from naphthalene, quinoline, isoquinoline, benzothiophene, phthalazine, cinnoline, tetrahydronaphthalene, tetrahydroquinoline, and tetrahydroisoquinoline, wherein the bicyclic ring system is substituted with exactly one substituent selected from COOH, CH₂COOH, CH(CH₃)COOH, SO₂NHCOR′, and CONHSO₂R′, wherein each R′ is independently C₁-C₆ alkyl, C₂-C₆ heteroalkyl, 2-methoxyethyl, 2′-(2-methoxyethoxy)ethyl, wherein each R′ can be optionally substituted with one or more groups selected from fluorine, C₁-C₄alkyl, C₁-C₄ heteroalkyl, and ═O.

45. The compound of claim 44, wherein the bicyclic ring is further substituted with 1 or more substituents selected from halo, CN, OR′, R′, or ═O, wherein each R′ is independently C₁-C₆ alkyl, C₂-C₆heteroalkyl, 2-methoxyethyl, 2′-(2-methoxyethoxy)ethyl.

46. The compound of claim 45, wherein R′ is further substituted with one or more substituents selected from F or C₁-C₃ alkyl.

47. The compound of claim 46, wherein Ring D and R₁₀ and R₁₁ taken together form naphthalene substituted with exactly one COOH; Ring A is phenyl and R₁ is COOR′, where R′ is C₁-C₅ alkyl; and R₂ and R₃ are not H or COOR′.

48. The compound of claim 46, wherein Ring D and R₁₀ and R₁₁ taken together form naphthalene substituted with exactly one COOH; Ring A is phenyl and one or more of R₁, R₂ and R₃ are COOR′, where R′ is C₁-C₅ alkyl, Rings B and C together are 3,3′-bipyridine.

49. The compound of claim 46, wherein Ring D and R₁₀ and R₁₁ taken together form naphthalene substituted with exactly one COOH; Ring A is phenyl and one or more of R₁, R₂ and R₃ is COOR′, wherein R′ is C₁-C₅ alkyl; and one or both of Ring B and Ring C are pyridazine.

50. The compound of claim 46, wherein Ring D and R₁₀ and R₁₁ taken together form naphthalene substituted with exactly one COOH; Ring A is phenyl; and one or more of R₁, R₂ and R₃ are COOR′, where R′ is C₁-C₅ alkyl; and Ring C and L₃ together are picolinamido.

51. The compound of claim 46, wherein Ring D and R₁₀ and R₁₁ form naphthalene; R₁₂ is carboxylic acid; Ring A is phenyl; L₁ is —CONH—, —NCO— or SOCH₂; L₂ is absent; and L₃ is —CONH—, —NCO— or —CH₂SO—.

52. The compound of claim 46, wherein Ring D and R₁₀ and R₁₁ form naphthalene; R₁₂ is carboxylic acid; Ring A is phenyl; L₁ is absent, and R₆ is independently selected from H, halogen or alkyl, X₇ is C or N.

53. The compound of claim 46, wherein Ring D and R₁₀ and R₁₁ form naphthalene; R₁₂ is carboxylic acid; Ring A is phenyl; L₁ is absent, R₆ is independently selected from H, halogen or alkyl; X₇ is C or N; and R₁, R₂, and R₃ are not —COR₁₇, COOR₁₇, —NH₂, —Cl, —F, or —CF₃, wherein R₁₇ is C₁₋₅ alkyl.

54. The compound of claim 1, wherein Ring D, R₁₀ and R₁₁ form naphthalene, the phenyl ring formed by R₁₀ and R₁₁ is independently substituted with exactly one of the following substituents: COOH, SO₂NHR′, wherein R′ is CO(C₁-C₆ alkyl) or CO(C₈-heteroalklyl) in which 2 carbons are replaced with oxygen.

55. The compound of claim 1, wherein Ring D, R₁₀, and R₁₁ form naphthalene; wherein the phenyl ring formed by R₁₀ and R₁₁ is independently substituted with one or more of COOH, SO₂NHR′, wherein R′ is CO(C₁-C₆ alkyl) or COC(C₈-heteroalklyl) (in which 2 carbons are replaced with oxygen), L₃ is selected from —CONH—, —NHCO—, CONHCH₂—, —NH—, —NHCH(CF₃)—, CON(CH₃)SO₂—, SO₂ N(CH₃)CO and —CH(CF₃)-NH—; Ring B is optionally substituted phenyl, pyridazine, pyridine, or thiophene; Ring C is optionally substituted phenyl, pyridazine, pyridine, thiophene, or furan, Ring A is optionally substituted phenyl, 1,3,4-thiadiazole, or piperidine.

56. The compound of claim 55, wherein Ring D is naphthalene substituted with a single COOH, Ring B is optionally substituted phenyl, pyridazine, pyridine, or thiophene; Ring C is optionally substituted phenyl, pyridazine, pyridine, thiophene, or furan, L₃ is —CONH—, —NHCO—, CONHCH₂—, —NH—, —NHCH(CF₃)—, —CONR′SO₂—, —SO₂NR′CO—, or —CCF₃—NH—; Ring A is optionally substituted phenyl, 1,3,4-thiadiazole, or piperidine; and wherein each R′ is independently C₁-C₆ alkyl, C₂-C₆ heteroalkyl, 2-methoxyethyl, 2′-(2-methoxyethoxy)ethyl wherein each R′ can be optionally substituted with one or more groups selected from fluorine, C₁-C₄alkyl, C₁-C₄ heteroalkyl, and ═O.

57. A compound selected from one or more of the following:

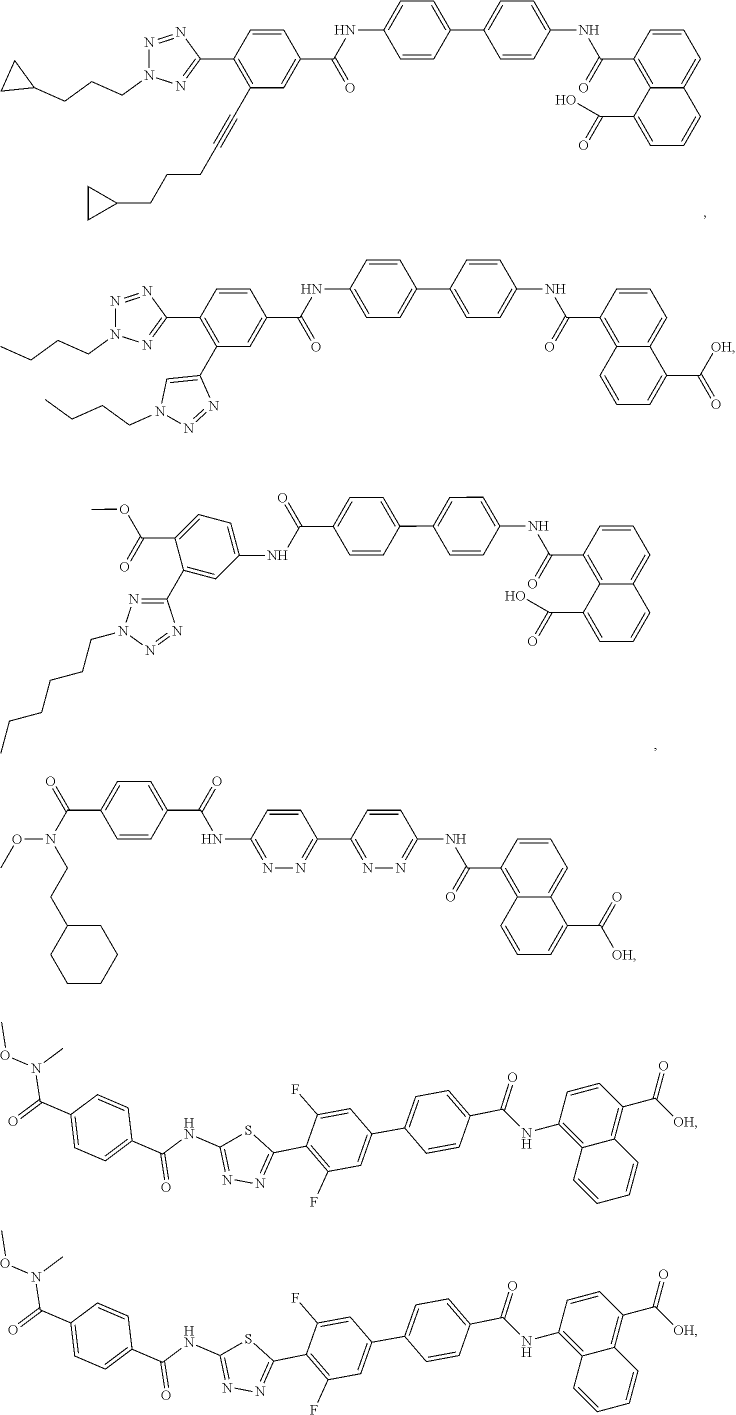

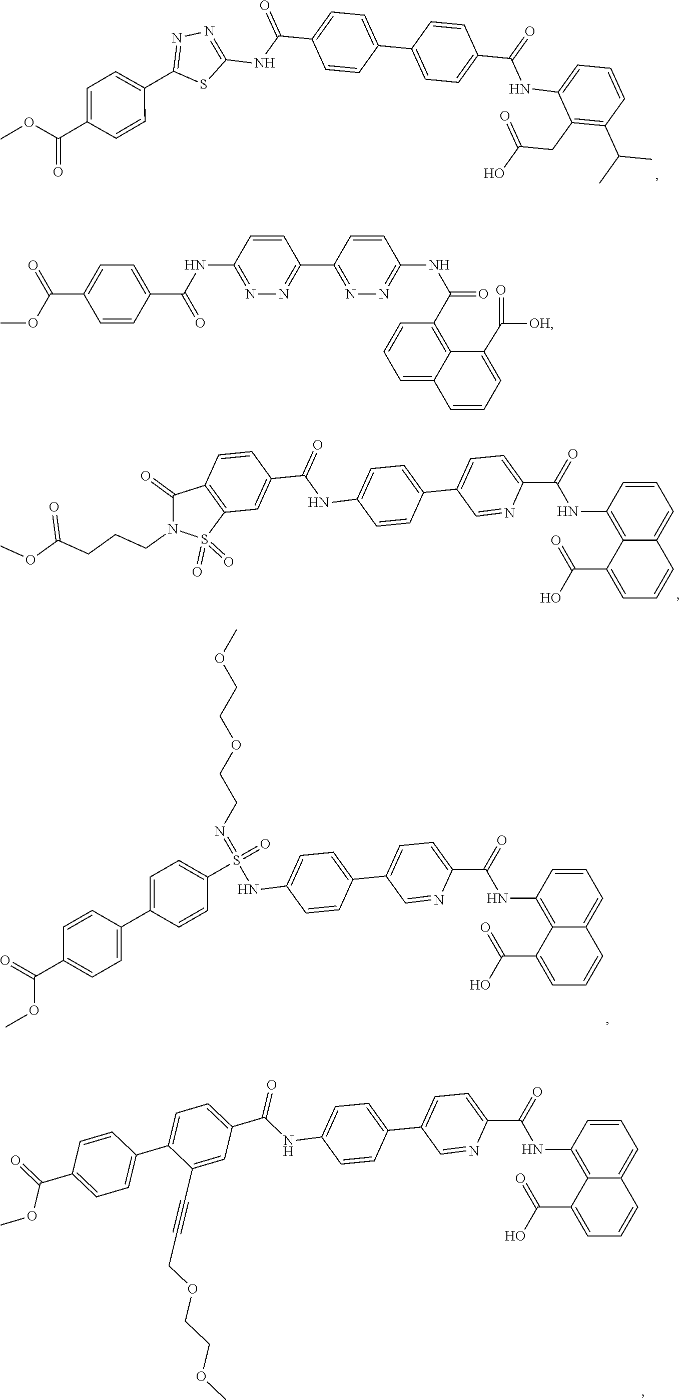

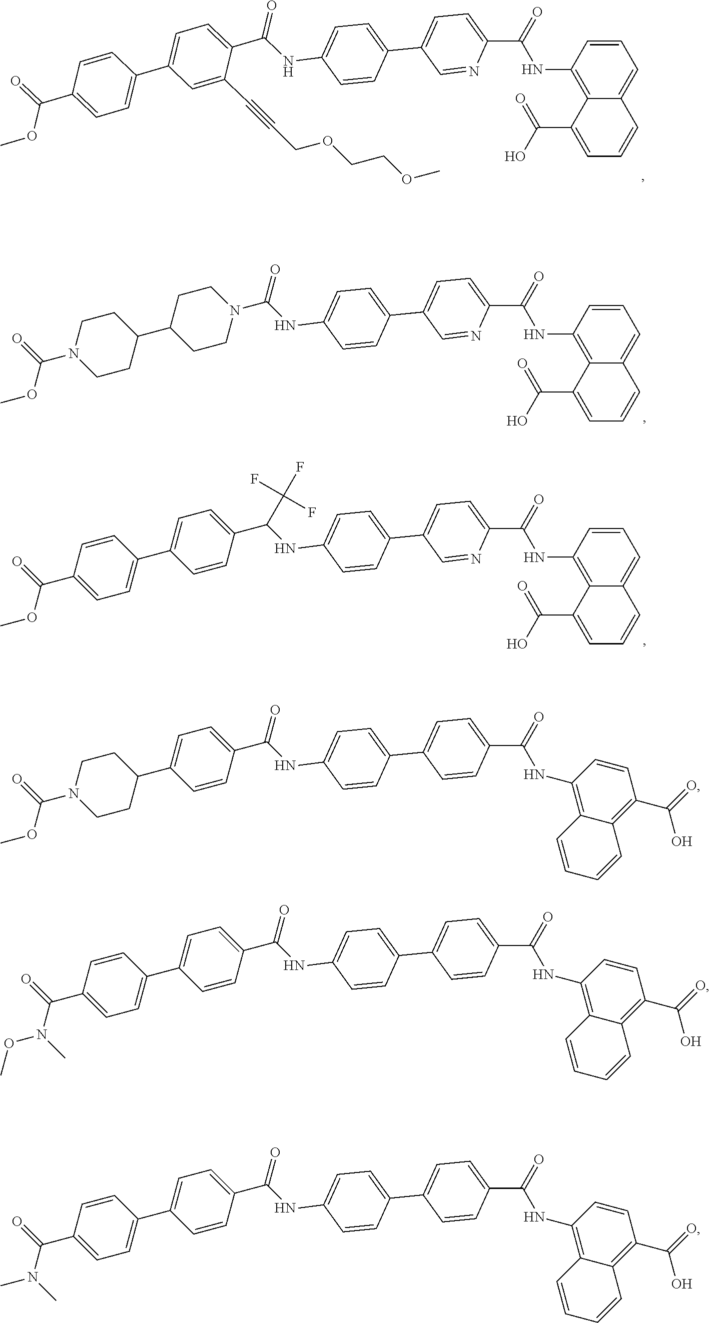

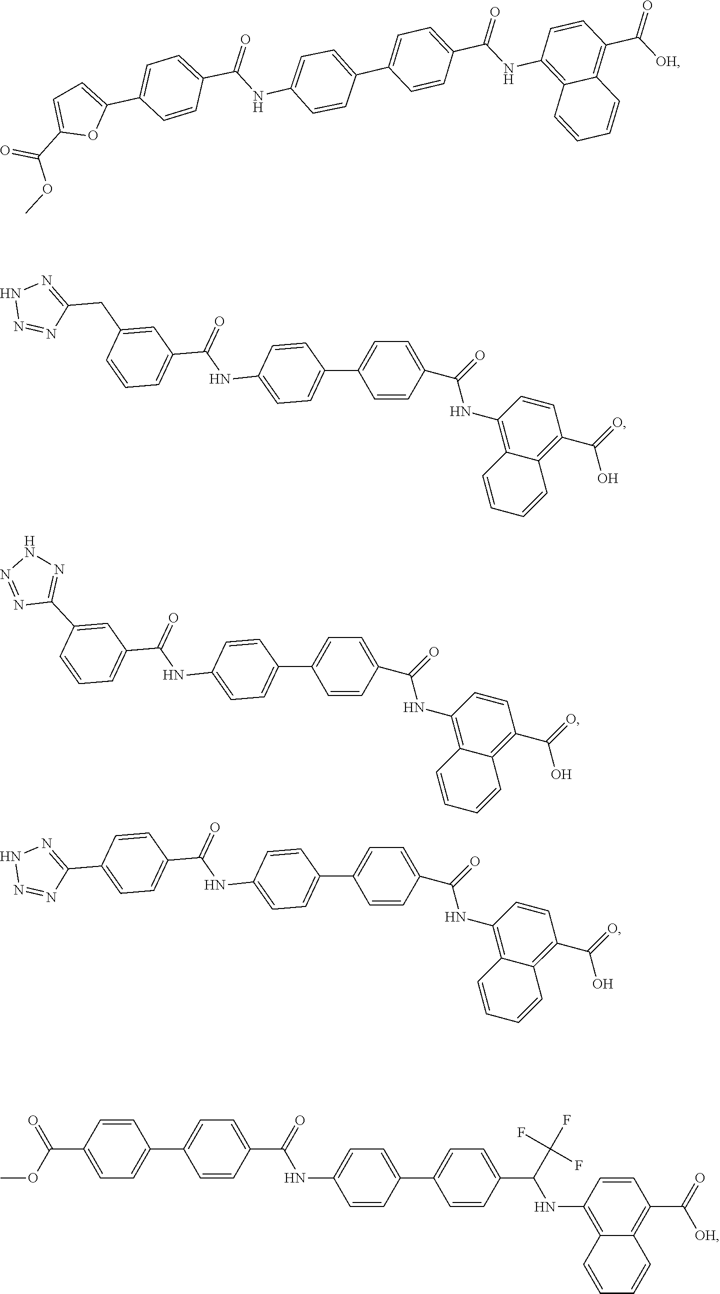 [SMILES: COC(=O)c1ccc(-c2ccc(C(=O)Nc3ccc(-c4ccc(C(=O)Nc5ccc(C(=O)O)c6ccccc56)cc4)cc3)cc2)o1 ; O=C(Nc1ccc(-c2ccc(C(=O)Nc3ccc(C(=O)O)c4ccccc34)cc2)cc1)c1cccc(Cc2nn[nH]n2)c1 ; O=C(Nc1ccc(-c2ccc(C(=O)Nc3ccc(C(=O)O)c4ccccc34)cc2)cc1)c1cccc(-c2nn[nH]n2)c1 ; O=C(Nc1ccc(-c2ccc(C(=O)Nc3ccc(C(=O)O)c4ccccc34)cc2)cc1)c1ccc(-c2nn[nH]n2)cc1 ; COC(=O)c1ccc(-c2ccc(C(=O)Nc3ccc(-c4ccc(C(Nc5ccc(C(=O)O)c6ccccc56)C(F)(F)F)cc4)cc3)cc2)cc1]

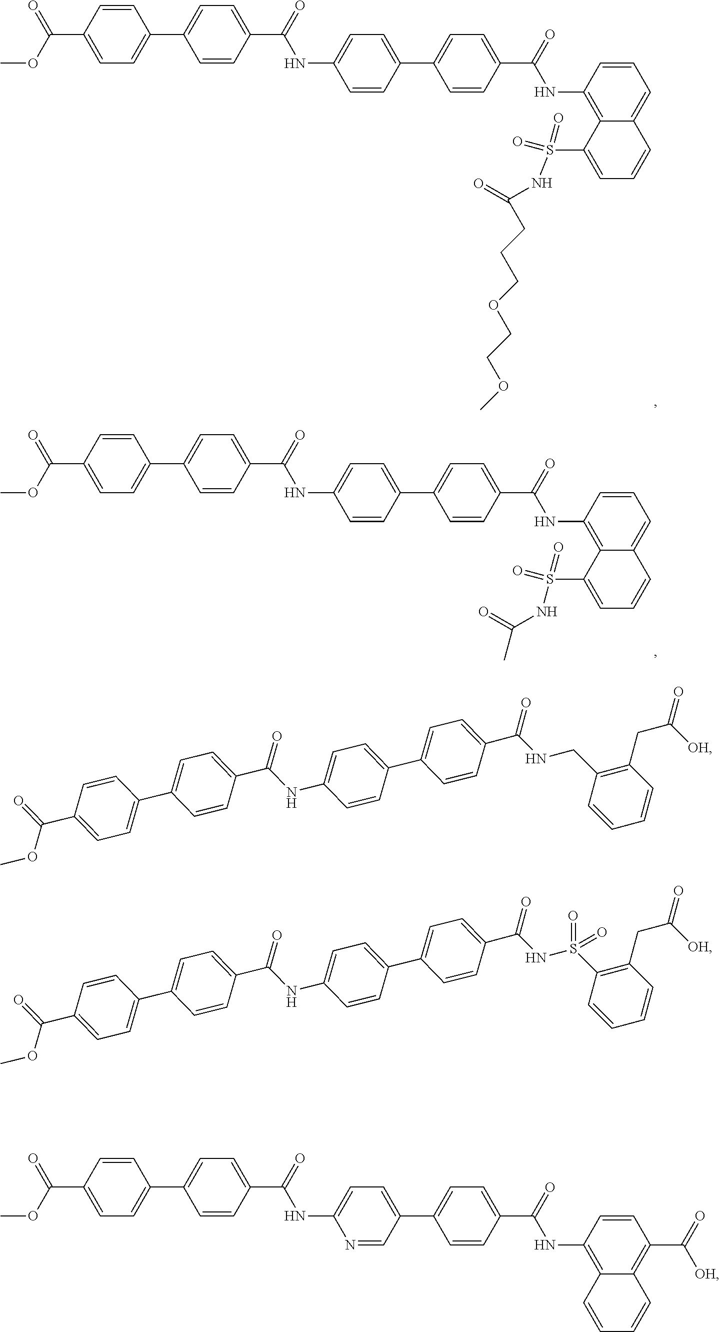 [SMILES: COCCOCCCC(=O)NS(=O)(=O)c1cccc2cccc(NC(=O)c3ccc(-c4ccc(NC(=O)c5ccc(-c6ccc(C(=O)OC)cc6)cc5)cc4)cc3)c12 ; COC(=O)c1ccc(-c2ccc(C(=O)Nc3ccc(-c4ccc(C(=O)Nc5cccc6cccc(S(=O)(=O)NC(C)=O)c56)cc4)cc3)cc2)cc1 ; COC(=O)c1ccc(-c2ccc(C(=O)Nc3ccc(-c4ccc(C(=O)NCc5ccccc5CC(=O)O)cc4)cc3)cc2)cc1 ; COC(=O)c1ccc(-c2ccc(C(=O)Nc3ccc(-c4ccc(C(=O)NS(=O)(=O)c5ccccc5CC(=O)O)cc4)cc3)cc2)cc1 ; COC(=O)c1ccc(-c2ccc(C(=O)Nc3ccc(-c4ccc(C(=O)Nc5ccc(C(=O)O)c6ccccc56)cc4)cn3)cc2)cc1]

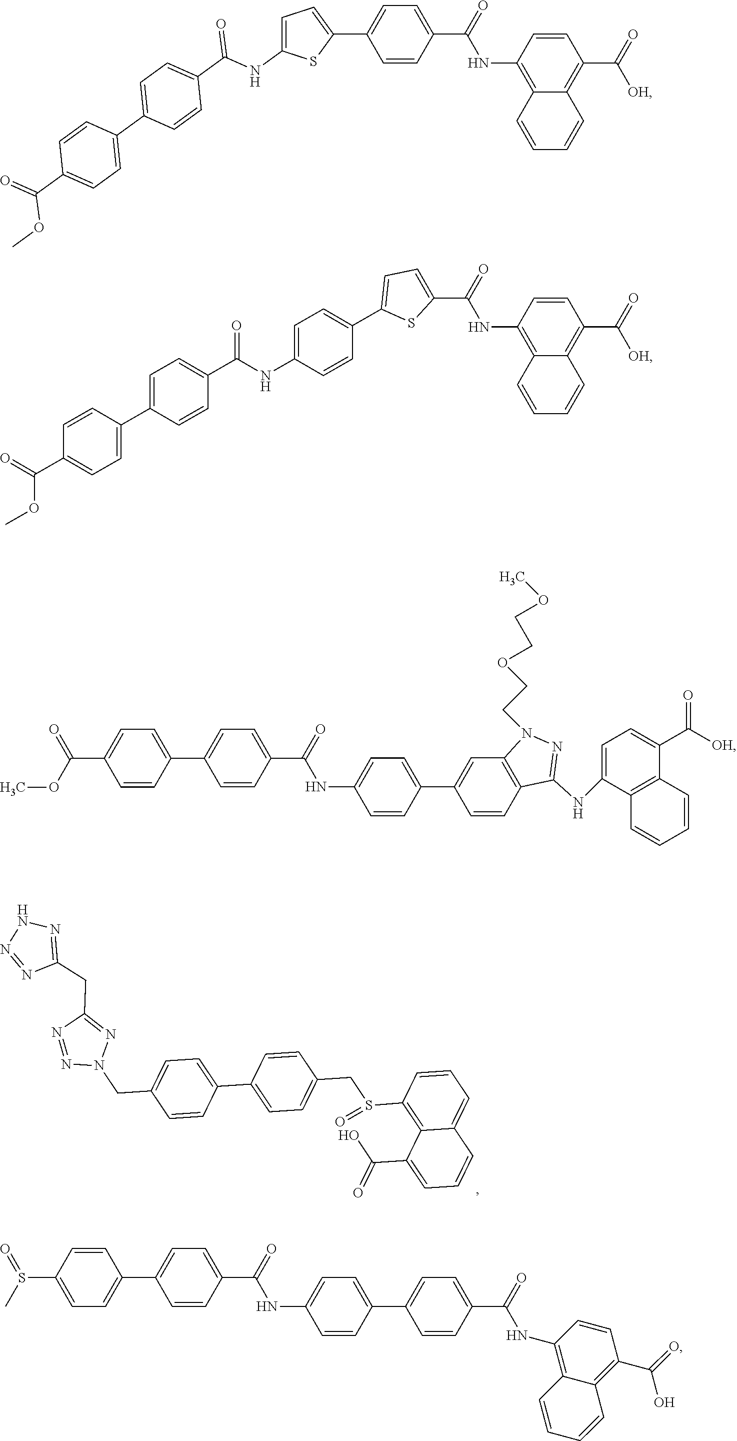 [SMILES: COC(=O)c1ccc(-c2ccc(C(=O)Nc3ccc(-c4ccc(C(=O)Nc5ccc(C(=O)O)c6ccccc56)cc4)s3)cc2)cc1 ; COC(=O)c1ccc(-c2ccc(C(=O)Nc3ccc(-c4ccc(C(=O)Nc5ccc(C(=O)O)c6ccccc56)s4)cc3)cc2)cc1 ; COCCOCCn1nc(Nc2ccc(C(=O)O)c3ccccc23)c2ccc(-c3ccc(NC(=O)c4ccc(-c5ccc(C(=O)OC)cc5)cc4)cc3)cc21 ; O=C(O)c1cccc2cccc(S(=O)Cc3ccc(-c4ccc(Cn5nnc(Cc6nn[nH]n6)n5)cc4)cc3)c12 ; CS(=O)c1ccc(-c2ccc(C(=O)Nc3ccc(-c4ccc(C(=O)Nc5ccc(C(=O)O)c6ccccc56)cc4)cc3)cc2)cc1]

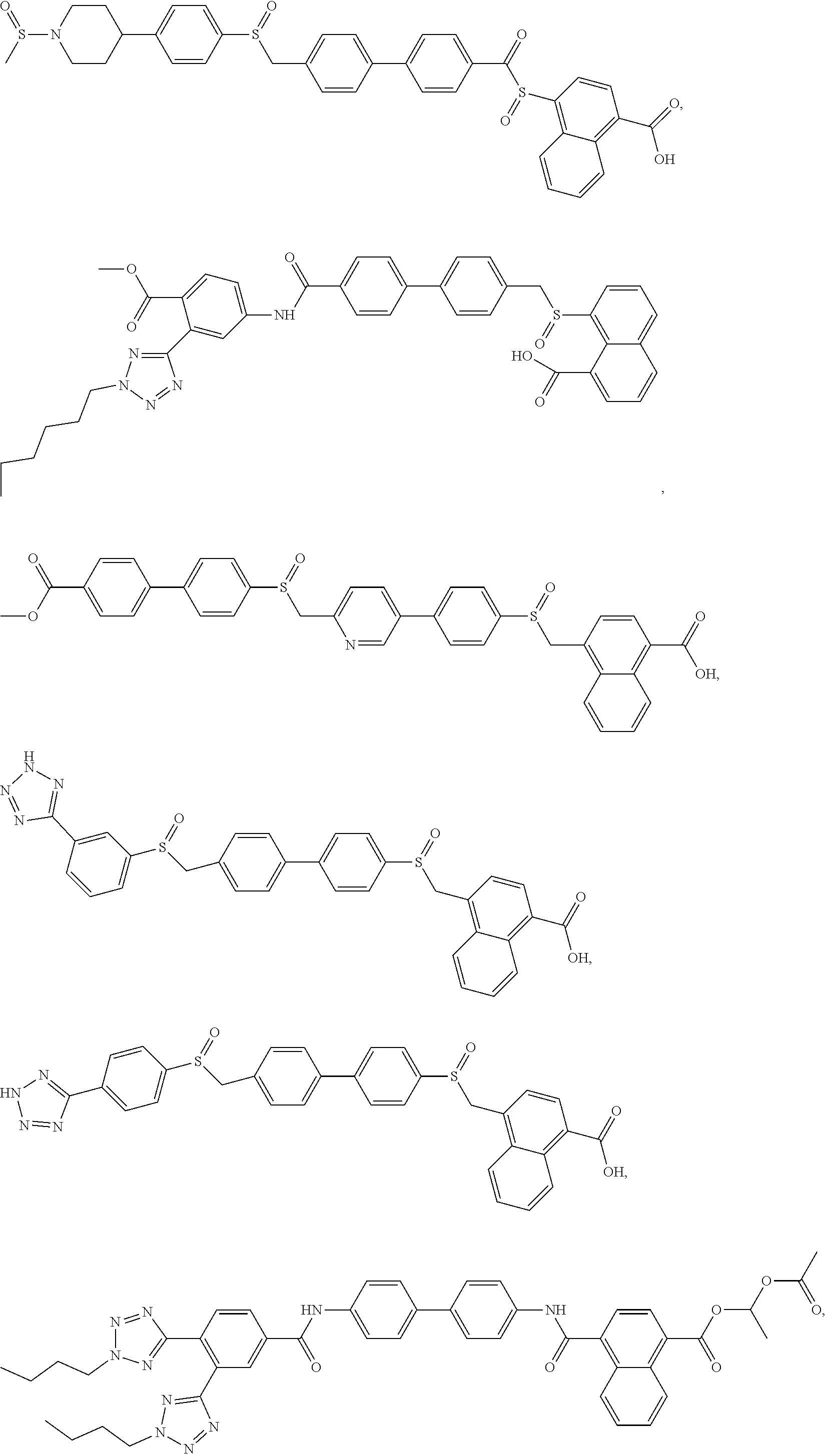

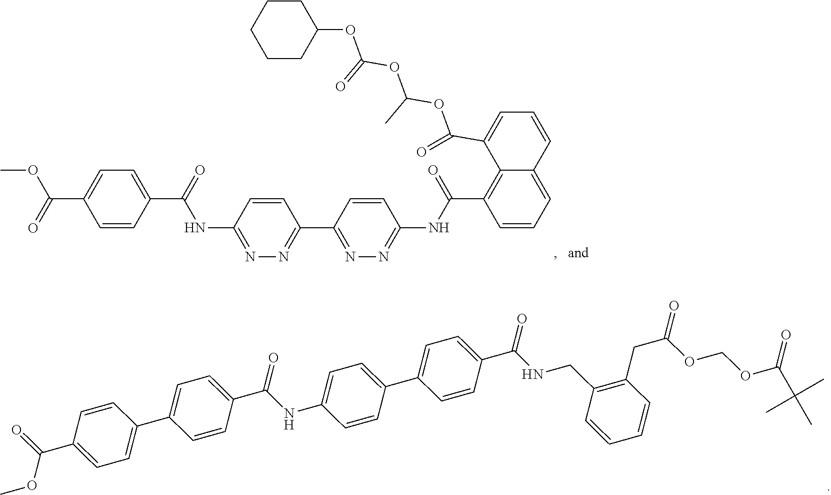 [SMILES: COC(=O)c1ccc(C(=O)Nc2ccc(-c3ccc(NC(=O)c4cccc5cccc(C(=O)OC(C)OC(=O)OC6CCCCC6)c45)nn3)nn2)cc1 ; COC(=O)c1ccc(-c2ccc(C(=O)Nc3ccc(-c4ccc(C(=O)NCc5ccccc5CC(=O)OCOC(=O)C(C)(C)C)cc4)cc3)cc2)cc1]

58. A compound selected from one or more of the following:
8-({4′-[3-(5-cyclopropylpent-1-yn-1-yl)-4-[2-(3-cyclopropylpropyl)-2H-
1,2,3,4-tetrazol-5-yl]benzamido]-[1,1′-biphenyl]-4-yl}carbamoyl)naphthalene-
1-carboxylic acid;
5-({4′-[3-(1-butyl-1H-1,2,3-triazol-4-yl)-4-(2-butyl-2H-1,2,3,4-tetrazol-5-
yl)benzamido]-[1,1′-biphenyl]-4-yl}carbamoyl)naphthalene-1-carboxylic acid;
8-[(4′-{[3-(2-hexyl-2H-1,2,3,4-tetrazol-5-yl)-
4(methoxycarbonyl)phenyl]carbamoyl}-[1,1′-biphenyl]-
4yl)carbamoyl]naphthalene-1-carboxylic acid;
5-[(6′-{4-[(2-cyclohexylethyl)(methoxy)carbamoyl]benzamido}-[3,3′-
bipyridazine]-6-yl)carbamoyl]naphthalene-1-carboxylic acid;
4-[3′,5′-difluoro-4′-(5-{4-[methoxy(methyl)carbamoyl]benzamido}-1,3,4-
thiadiazol-2-yl)-[1,1′-biphenyl]-4-amido]naphthalene-1-carboxylic acid;
5-[(6′-{4-[4-(2-ethoxy-2-oxoethyl)-1H-1,2,3-triazol-1-yl]benzamido}-[3,3′-
bipyridine]-6-yl)carbamoyl]naphthalene-1-carboxylic acid;
2-{2-[4′-({5-[4-(methoxycarbonyl)phenyl]-1,3,4-thiadiazol-2-yl}carbamoyl)-
[1,1′-biphenyl]-4-amido]-6-(propan-2-yl)phenyl}acetic acid;
8-({6′-[4-(methoxycarbonyl)benzamido]-[3,3′-bipyridazine]-6-
yl}carbamoyl)naphthalene-1-carboxylic acid;
8-(5-{4-[2-(4-methoxy-4-oxobutyl)-1,1,3-trioxo-2,3-dihydro-1lambda6,2-
benzothiazole-6-amido]phenyl}pyridine-2-amido)naphthalene-1-carboxylic
acid;
8-[5-(4-{N-[2-(2-methoxyethoxy)ethyl]4′-(methoxycarbonyl)-[1,1′-biphenyl]-4-
sulfonoimidamido}phenyl)pyridine-2-amido]naphthalene-1-carboxylic acid;
8-(5-{4-[4′-(methoxycarbonyl)-2-[3-(2-methoxyethoxy)prop-1-yn-1-yl]-[1,1′-
biphenyl]-4-amido]phenyl}pyridine-2-amido)naphthalene-1-carboxylic acid;
8-(5-{4-[4′-(methoxycarbonyl)-2′-[3-(2-methoxyethoxy)prop-1-yn-1-yl]-[1,1′-
biphenyl]-4-amido]phenyl}pyridine-2-amido)naphthalene-1-carboxylic acid;
8-[5-(4-{[1′-(methoxycarbonyl)-[4,4′-bipiperidine]-1-
carbonyl]amino}phenyl)pyridine-2-amido]naphthalene-1-carboxylic acid;
8-(5-(4-((2,2,2-trifluoro-1-(4′-(methoxycarbonyl)-[1,1′-biphenyl]-4-
yl)ethyl)amino)phenyl)picolinamido)-1-naphthoic acid;
4-(4′-{4-[1-(methoxycarbonyl)piperidin-4-yl]benzamido}-[1,1′-biphenyl]-4-
amido)naphthalene-1-carboxylic acid;
4-(4′-{4′-[methoxy(methyl)carbamoyl]-[1,1′-biphenyl]-4-amido}-[1,1′-
biphenyl]-4-amido)naphthalene-1-carboxylic acid;
4-{4′-[4′-(dimethylcarbamoyl)-[1,1′-biphenyl]-4-amido]-[1,1′-biphenyl]-4-
amido}naphthalene-1-carboxylic acid;
4-(4′-{4-[5-(methoxycarbonyl)furan-2-yl]benzamido}-[1,1′-biphenyl]-4-
amido)naphthalene-1-carboxylic acid;
4-(4′-{3-[(2H-1,2,3,4-tetrazol-5-yl)methyl]benzamido}-[1,1′-biphenyl]-4-
amido)naphthalene-1-carboxylic acid;
4-{4′-[3-(2H-1,2,3,4-tetrazol-5-yl)benzamido]-[1,1′-biphenyl]-4-
amido}naphthalene-1-carboxylic acid;
4-{4′-[4-(2H-1,2,3,4-tetrazol-5-yl)benzamido]-[1,1′-biphenyl]-4-
amido}naphthalene-1-carboxylic acid;
4-[(2,2,2-trifluoro-1-{4′-[4′-(methoxycarbonyl)-[1,1′-biphenyl]-4-amido]-[1,1′-
biphenyl]-4-yl}ethyl)amino]naphthalene-1-carboxylic acid;
methyl 4′-({4′-[(8-{[4-(2-methoxyethoxy)butanamido]sulfonyl}naphthalen-1-
yl)carbamoyl]-[1,1′-biphenyl]-4-yl}carbamoyl)-[1,1′-biphenyl]-4-carboxylate;
methyl4′-[(4′-{[8-(acetamidosulfonyl)naphthalen-1-yl]carbamoyl}-[1,1′-
biphenyl]-4-yl)carbamoyl]-[1,1′-biphenyl]-4-carboxylate;
2-{2-[({4′-[4′-(methoxycarbonyl)-[1,1′-biphenyl]-4-amido]-[1,1′-biphenyl]-4-
yl}formamido)methyl]phenyl}acetic acid;
2-{2-[({4′-[4′-(methoxycarbonyl)-[1,1′-biphenyl]-4-amido]-[1,1′-biphenyl]-4-
yl}formamido)sulfonyl]phenyl}acetic acid;
4-(4-{6-[4′-(methoxycarbonyl)-[1,1′-biphenyl]-4-amido]pyridin-3-
yl}benzamido)naphthalene-1-carboxylic acid;
4-(4-{5-[4′-(methoxycarbonyl)-[1,1′-biphenyl]-4-amido]thiophen-2-
yl}benzamido)naphthalene-1-carboxylic acid;
4-(5-{4-[4′-(methoxycarbonyl)-[1,1′-biphenyl]-4-amido]phenyl}thiophene-2-
amido)naphthalene-1-carboxylic acid and,
4-{N-[2-(2-methoxyethoxy)ethyl]4′-[4′-(methoxycarbonyl)-[1,1′-biphenyl]-4-
amido]-[1,1′-biphenyl]-4-sulfonoimidamido}naphthalene-1-carboxylic acid.
8-(((4′-((5-((2H-tetrazol-5-yl)methyl)-2H-tetrazol-2-yl)methyl)-[1,1′-biphenyl]-
4-yl)methyl)sulfinyl)-1-naphthoic acid
4-(4′-(4′-(methylsulfinyl)-[1,1′-biphenyl]-4-carboxamido)-[1,1′-biphenyl]-4-
carboxamido)-1-naphthoic acid
4-(((4′-(((4-(1-(methoxycarbonyl)piperidin-4-yl)phenyl)sulfinyl)methyl)-[1,1′-
biphenyl]-4-yl)methyl)sulfinyl)-1-naphthoic acid
4-(((4′-(((4-(1-(methoxycarbonyl)piperidin-4-yl)phenyl)sulfinyl)methyl)-[1,1′-
biphenyl]-4-yl)methyl)sulfinyl)-1-naphthoic acid
4-(((4′-(((3-(2H-tetrazol-5-yl)phenyl)sulfinyl)methyl)-[1,1′-biphenyl]-4-
yl)sulfinyl)methyl)-1-naphthoic acid
4-(((4′-(((4-(2H-tetrazol-5-yl)phenyl)sulfinyl)methyl)-[1,1′-biphenyl]-4-
yl)sulfinyl)methyl)-1-naphthoic acid
8-(((4′-((3-(2-hexyl-2H-tetrazol-5-yl)-4-(methoxycarbonyl)phenyl)carbamoyl)-
[1,1′-biphenyl]-4-yl)methyl)sulfinyl)-1-naphthoic acid

59. The compound of claim 1, further comprising a prodrug, wherein the COOH of R₁₀, R₁₁, or R₁₂ of Ring D is replaced with an ester group selected from C(O)O—CH₂C(CH₃)₃ and C(O)O—CH(CH₃)-O-cyclohexyl.

60. The compound of claim 1, further comprising a prodrug, wherein when R₁₀ and R₁₁ taken together, along with the two intervening atoms to which they are attached, form an optionally substituted five or six-membered aromatic, aliphatic heteroaromatic, or heteroaliphatic ring, so that ring D, R₁₀ and R₁₁ taken together form a bicyclic ring system, wherein the bicyclic ring system is substituted with exactly one COOH, the COOH is replaced with an ester group selected from C(O)O—CH₂C(CH₃)₃ and C(O)O—CH(CH₃)—O-cyclohexyl.

61. The compound according to claim 43, 44, or 47-58, further comprising a prodrug wherein the COOH of ring D is replaced with an ester group selected from C(O)O—CH₂C(CH₃)₃ and C(O)O—CH(CH₃)—O-cyclohexyl.

62. A prodrug of claim 1, selected from one or more of the following:

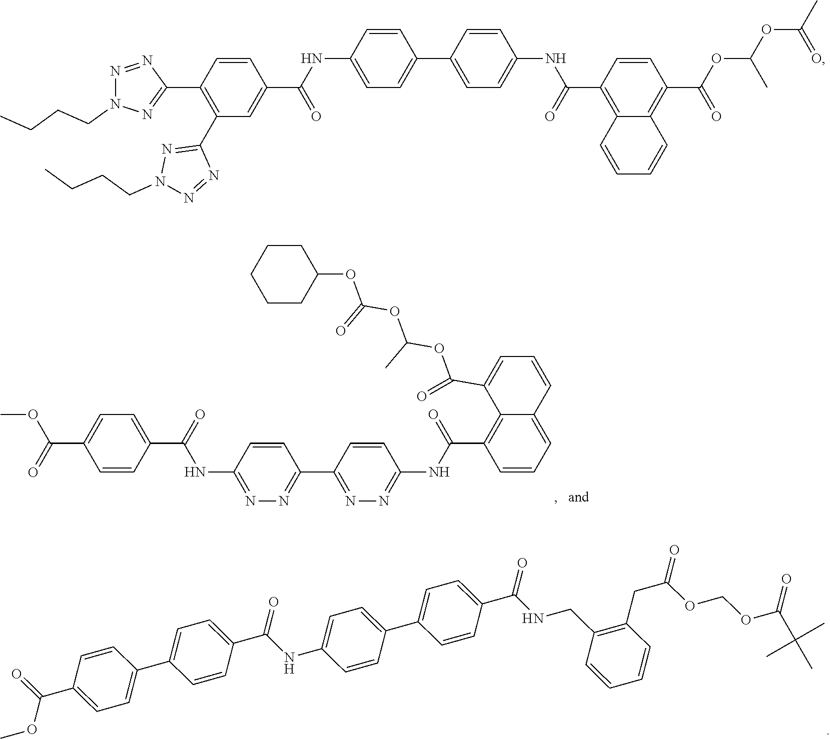 [SMILES: CCCCn1nnc(-c2ccc(C(=O)Nc3ccc(-c4ccc(NC(=O)c5ccc(C(=O)OC(C)OC(C)=O)c6ccccc56)cc4)cc3)cc2-c2nnn(CCCC)n2)n1 ; COC(=O)c1ccc(C(=O)Nc2ccc(-c3ccc(NC(=O)c4cccc5cccc(C(=O)OC(C)OC(=O)OC6CCCCC6)c45)nn3)nn2)cc1 ; COC(=O)c1ccc(-c2ccc(C(=O)Nc3ccc(-c4ccc(C(=O)NCc5ccccc5CC(=O)OCOC(=O)C(C)(C)C)cc4)cc3)cc2)cc1]

62. A pharmaceutical composition comprising a compound of claims 1-61, or a pharmaceutically acceptable salt thereof, and a pharmaceutically acceptable excipient.

63. A method of treating, ameliorating, or preventing an autoimmune disease, or other immune-related diseases, atherosclerotic disorders, neurodegenerative disorders, inflammatory disease, prevention of transplantation rejection, and treatment of malignancies, comprising administering to a subject in need one or more compounds of claims 1-62.

64. The method of claim 63, wherein the disease is selected from one or more of systemic lupus erythematosus (SLE), rheumatoid arthritis, ankylosing spondylitis, lupus nephritis, Goodpasture's disease, Sjögren's syndrome, polymyositis, dermatomyositis, psoriasis, temporal arteritis, Churg-Strauss syndrome, multiple sclerosis, Guillain-Barré syndrome, transverse myelitis, myasthenia gravis, Addison's disease, thyroiditis, coeliac disease, ulcerative colitis, sarcoidosis, hemolytic anemia, idiopathic thrombocytopenic purpura, Behçet's disease, Alzheimer's disease, traumatic brain injury, chronic traumatic encephalitis, Parkinson's disease, angina, pectoris, myocardial infarction, primary biliary cirrhosis Crohn's disease, type 1 diabetes, Juvenile diabetes, autoimmune diabetes, acute and chronic rejection in bone marrow transplantation, graft versus host disease, acute and chronic rejection in orthotopic and heterotopic solid organ transplants, and prevention of rejection of bone marrow transplants.

65. A method of modulating tumor necrosis factor (TNF) super family comprising administering to a patient in need of treatment one or more compounds of claims 1-62.

66. The method of claim 65, wherein the compound or pharmaceutical composition, modulates one or more interactions selected from CD-40-CD40L, CD152,CD27-CD70, CD137(4-1BB)-4-1BBL, HVEM-LIGHT(CD258), CD30-CD30L, GITR-GITRL, BAFF-R(CD268)-BAFF(CD257), RANK(CD265)-RANKL(CD254), OX40(CD 134)-OX40L(CD252), and combinations thereof.

67. A compound of Formula II 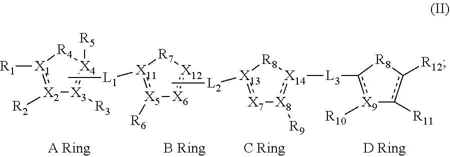
or a pharmaceutically acceptable salt, ester, prodrug, hydrate and tautomer thereof, wherein:
Rings A and D are optionally substituted 5- or 6-membered aromatic or heteroaromatic rings with 2-4 nitrogens;
Rings B and C are optionally substituted 5- or 6-membered aromatic or heteroaromatic rings with 0-4 nitrogens;
X₁, X₂, X₃, X₄ are each separately and independently selected from the group consisting of C, or N;
L₁ is a bond, (CH₂) where (n=1-3), NH—, 1,2,3-triazole linked at 1 and 4, or 5-alkyl-tetrazole linked at the 2 position and the alkyl terminus (alkyl is 0-3 carbons);
L₂ is a bond, (CH₂)n where n is 1 to 5, CH(OH), C(CH₃)₂, —CH(OH)—, —CH₂NH—, benzene-1,2-diyl, benzene-1,3-diyl, benzene-1,4-diyl, pyridine-3,5-diyl;
R₁ is H, F, COOR₁₄, CONR₁₄, OR₁₅, SO₂R₁₄, SO₂NR₁₄, COR₁₅, tetrazole linked through its carbon, or CH₂-tetrazole linked through its carbon;
R₂ is H, F, COOR₁₄, CONR₁₄(OR₁₅), SO₂R₁₄, SO₂NR₁₄COR₁₅, tetrazole linked through its carbon, CH₂-tetrazole linked through its carbon; with the proviso that R₁ and R₂ cannot both be H or F;
R₃ is H, For absent;
R₄ is selected from the group of CH, CH═CR₁₆, CH═CH, S, O, N, N═CH, CH═N, N═N, and CH₂CH₂, wherein R₁₆ is OH, OCHF₂, NHCOR₁₄, H, F, CH═N, or N═CH;
R₆ is H, F, methyl or absent;
R₇ is CH, N, CR₆═CH, or N═CH, optionally substituted with methyl on carbon atoms;
R₈ is CH, N, CH═CH, CH═N, or N═CH, optionally substituted with methyl on carbon atoms;
R₉ is H, F, Cl, methyl, NH₂ or absent;
R₁₀ is H, CH₃, CH₂COOH, CH₂SO₂NHCOR₁₇, SO₂NHCOR₁₇, or tetrazole linked from its carbon (5 position); R₁₁ is H, COOH, CH₂COOH, CH₂SO₂NHCOR₁₇, SO₂NHCOR₁₇, or tetrazole linked from its carbon (5 position); or
R₁₀ and R₁₁ optionally form an aromatic ring fused to ring D, including without limitation a fused benzene, or pyridine ring.
R₁₂ is H or SO₂NHCOR₁₇;
R₁₃ is CH═CH, CH═C(COOH), CH═C(CH₂COOH), CH═C(SO₂NHCOR₁₆), CH═C(CH₂SO₂NHCOR₁₇);
R₁₄ and R₁₅ are, independently, C₁-C₁₀ alkyl, C₃-C₈ cycloalkyl, or C₃-C₆ cycloalkyl linked through bonds or 1-8 carbon alkyl chains {(CH₂)n with n=1 to 8} which can be optionally substituted with 1-3 fluorine atoms;
and R₁₇ is H, CH₃, N(CH₃)₂, (CH₂CH₂O)ₙCH₃, NCH₃((CH₂CH₂O)ₙCH₃, where n=1 to 6.

68. The compound of claim 67, wherein Ring A is selected from optionally substituted phenyl, benzene, pyridine, triazole and tetrazole; Ring B is selected from optionally substituted diazole, triazole, tetrazole, pyridazine, pyrimidine, benzene, pyridine, piperidine, or piperazine; Ring C is selected from optionally substituted 1,2,3-triazole, tetrazole, benzene pyridine, pyridazine, 1,2,4-triazine, piperazine, and piperidine; and Ring D is selected from optionally substituted benzene, pyridine and thiophene;

69. The compound of claims 67 and 68, wherein R₁₀ and R₁₁ with ring D form a bicyclic aromatic ring selected from napthalene, quinoline, isoquinoline, and benzothiophene.

70. The compound of claim 69 wherein the bicyclic aromatic ring is naphthalene.

71. The compound of claim 70, wherein Ring A is optionally substituted from the group selected from OH, SO₂NR′₂, SO₂R′, COR′, COOR′, CON(R′)₂, CON(OR′)R′, NCOR′, tetrazole, triazole, and alkyl-heteroaryl or phenyl, wherein R′ is selected from C₁-C₅ alkyl, C₃-C₁₀ heteroalkyl, and wherein the heteroatoms are 1-3 oxygens, C₃-C₆ cycloalkyl, optionally substituted with 1-3 fluorine atoms.

72. The compound of claim 71, wherein Ring A is optionally substituted with phenyl.

73. The compound of claim 72, wherein the phenyl is optionally substituted with OH, NHCOCH₃, SOCH₃, NHCH₃, COR′, COOR′, or CON(R′)₂, where R′ is independently selected from C₁-C₆ alkyl or C₁-C₃alkoxy.

74. The compound of claim 71, wherein Ring A is substituted with OH and/or COR′.

75. The compound of claim 71, wherein Ring A is a tetrazole substituted with an alkyl-heteroaryl.

76. The compound of claims 71 and 75, wherein the alkyl-heteroaryl is 5-ethyl-2H-tetrazole.

77. The compound of claims 67-76, wherein L₁ can optionally combine with R₄ to form a heteroaromatic ring fused to ring A which can be optionally substituted from the group selected from OH, SO₂NR′₂, SO₂R′, COR′, COOR′, CON(R′)₂, CON(OR′)R′, NHCOR′, tetrazole, triazole, and alkyl-heteroaryl.

78. The compound of claims 67-77, wherein R₁₃ forms a ring with Ring D to form a bicyclic aromatic ring.

79. The compound of claim 78, wherein the bicyclic aromatic ring is naphthalene, isoquinoline, or benzthiophene.

80. The compound of claims 67-79, wherein when Ring B is a 6-membered ring, the relative positions of the L₁ and L₂ links to ring B can be 1,2; 1,3; or 1,4.

81. The compound of claim 67-79, wherein if Ring B is a tetrazole, L₁ is linked to the 2 position and L₂ is linked to the 5 position of the tetrazole.

82. The compound of claims 67-81, wherein the relative positions of the L₂ and L₃ links to ring C can be 1,2; 1,3; 3, 5; 3, 6; 2,5; 3,6; or 1,4.

83. The compound of claim 82, wherein if Ring C is a 6-membered ring, the relative positions of the L₂ and L₃ links to Ring C can be 1,2; 1,3; or 1,4.

84. The compound of claim 82, wherein if Ring C is 1,2,3-triazole L₃ is linked to the 1 position and L₂ is linked to the 4 position of the 1,2,3-triazole.

85. The compound of claim 82, wherein if Ring C is a tetrazole L₃ is linked to the 2 position and L₂ is linked to the 5 position.

86. The compound of claims 67-85, wherein R₄, L₁, Ring B, and Ring C contain at least 4 to 8 aromatic nitrogen atoms, with at least 1 pair of adjacent aromatic nitrogen atoms without substituents (N═N or N—NH).

87. The compound of claims 67-86, wherein R₄ is N, X₁ is C, X₂, X₃ and X₄ are N.

88. The compound of claims 67-86, wherein R₄ is C═C, and X₁, X₂, X₃, X₄ are C.

89. The compound of claims 67-86, wherein R₄ is N, X₁ and X₂ are N, and X₃ and X₄ are C.

90. The compound of claims 67-86, wherein R₄ is N, X₁, X₂, X₃ are N, and X₄ is C.

91. The compound of claims 67-86, wherein R₄ is N, X₁ and X₄ are N, X₂ and X₃, are C.

92. The compound of claims 67-86, wherein R₄ is C, X₁, X₂, X₃ are N, and X₄, is C.

93. The compound of claims 67-92, wherein only one acidic group is linked to Ring D, R₁₀, R₁₁, R₁₂, or R₁₃ and wherein the acidic group is ionizable to anion at pH 7.4.

94. The compound of claim 93, wherein the acid group is selected from COOH, CH₂COOH, SO₂NHCOR₁₇, CH₂SO₂NHCOR₁₇, or tetrazole.

95. The compound of claim 67, wherein Ll is a bond and Rings A and B are fused to one another to form a heteroaromatic bicycle that is optionally substituted.

96. The compound of claim 95, wherein the bicycle is benzimidazole.

97. The compound of claim 95 or 96, wherein the heteraromatic bicycle is substituted with SO₂R′ and R′ is selected from C₁-C₆ alkyl or C₁-C₃ alkoxy.

98. A compound selected from one or more of the following:

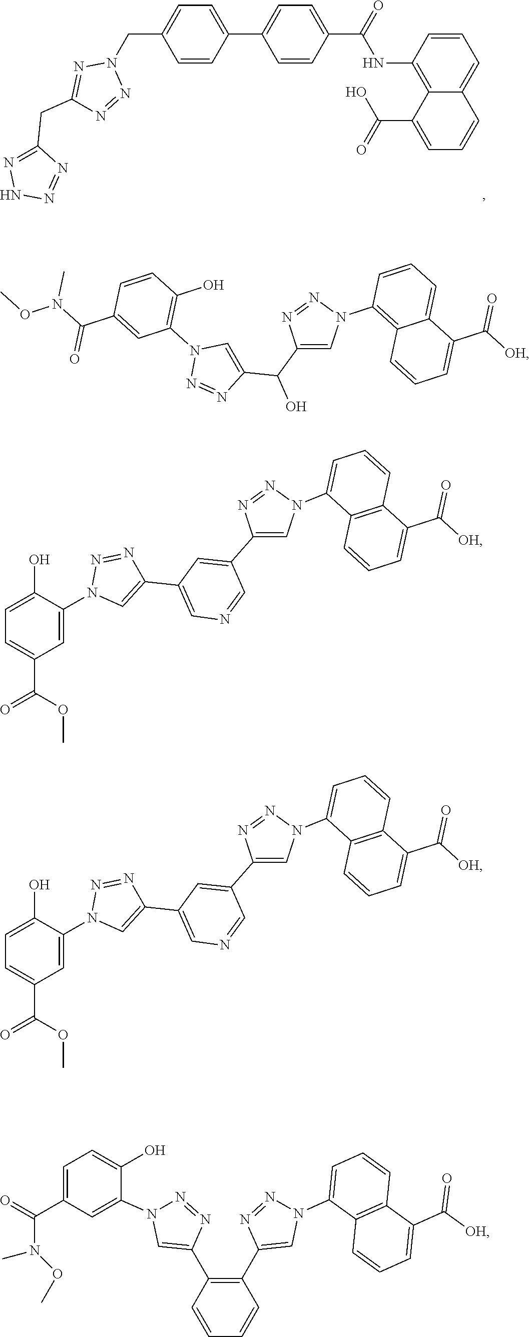 [SMILES: O=C(Nc1cccc2cccc(C(=O)O)c12)c1ccc(-c2ccc(Cn3nnc(Cc4nn[nH]n4)n3)cc2)cc1 ; CON(C)C(=O)c1ccc(O)c(-n2cc(C(O)c3cn(-c4cccc5c(C(=O)O)cccc45)nn3)nn2)c1 ; COC(=O)c1ccc(O)c(-n2cc(-c3cncc(-c4cn(-c5cccc6c(C(=O)O)cccc56)nn4)c3)nn2)c1 ; COC(=O)c1ccc(O)c(-n2cc(-c3cncc(-c4cn(-c5cccc6c(C(=O)O)cccc56)nn4)c3)nn2)c1 ; CON(C)C(=O)c1ccc(O)c(-n2cc(-c3ccccc3-c3cn(-c4cccc5c(C(=O)O)cccc45)nn3)nn2)c1]

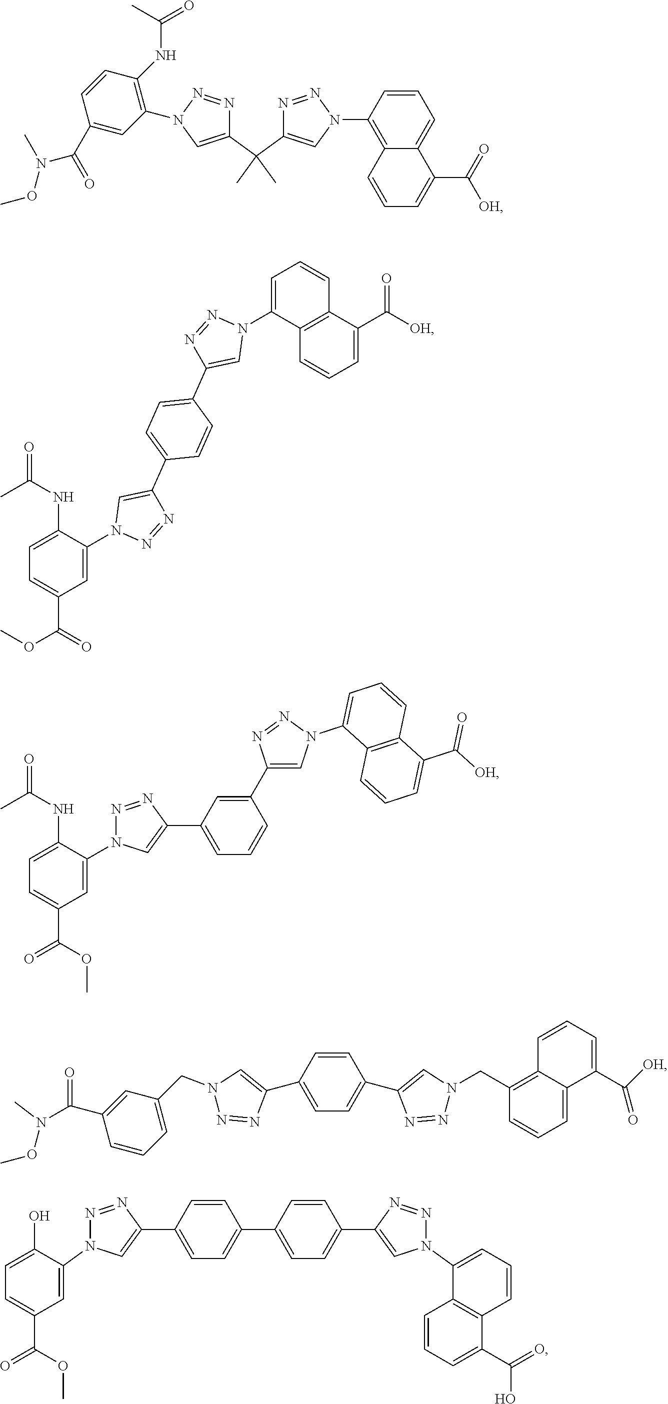 [SMILES: CON(C)C(=O)c1ccc(NC(C)=O)c(-n2cc(C(C)(C)c3cn(-c4cccc5c(C(=O)O)cccc45)nn3)nn2)c1 ; COC(=O)c1ccc(NC(C)=O)c(-n2cc(-c3ccc(-c4cn(-c5cccc6c(C(=O)O)cccc56)nn4)cc3)nn2)c1 ; COC(=O)c1ccc(NC(C)=O)c(-n2cc(-c3cccc(-c4cn(-c5cccc6c(C(=O)O)cccc56)nn4)c3)nn2)c1 ; CON(C)C(=O)c1cccc(Cn2cc(-c3ccc(-c4cn(Cc5cccc6c(C(=O)O)cccc56)nn4)cc3)nn2)c1 ; COC(=O)c1ccc(O)c(-n2cc(-c3ccc(-c4ccc(-c5cn(-c6cccc7c(C(=O)O)cccc67)nn5)cc4)cc3)nn2)c1]

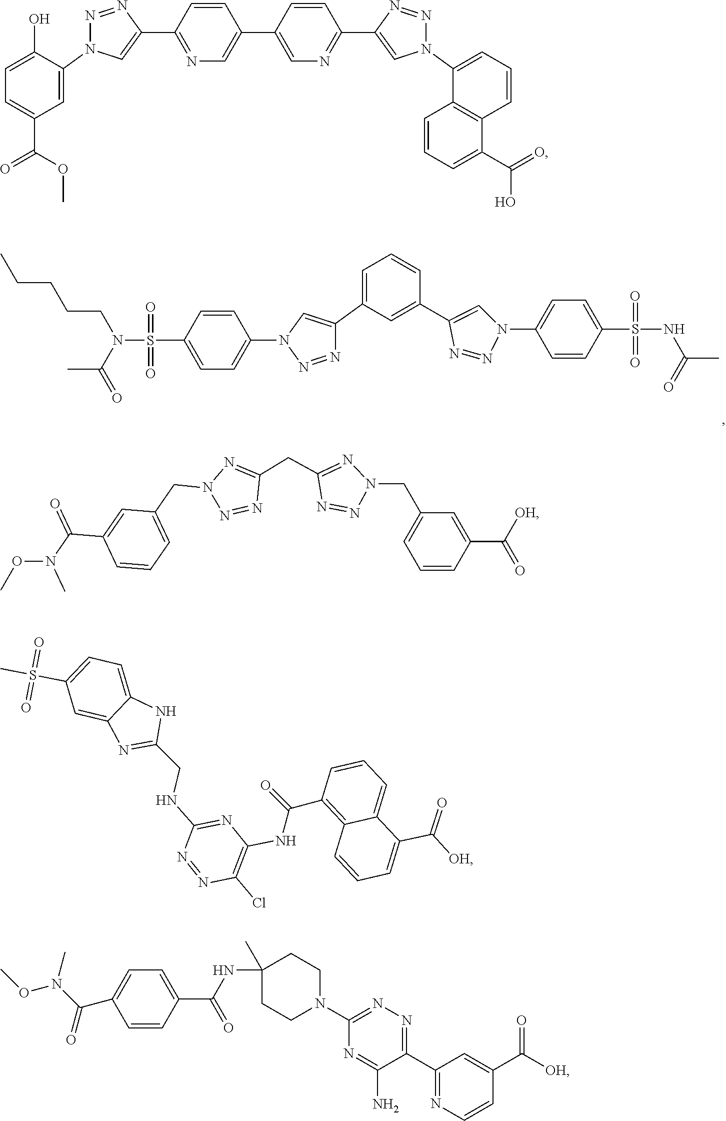

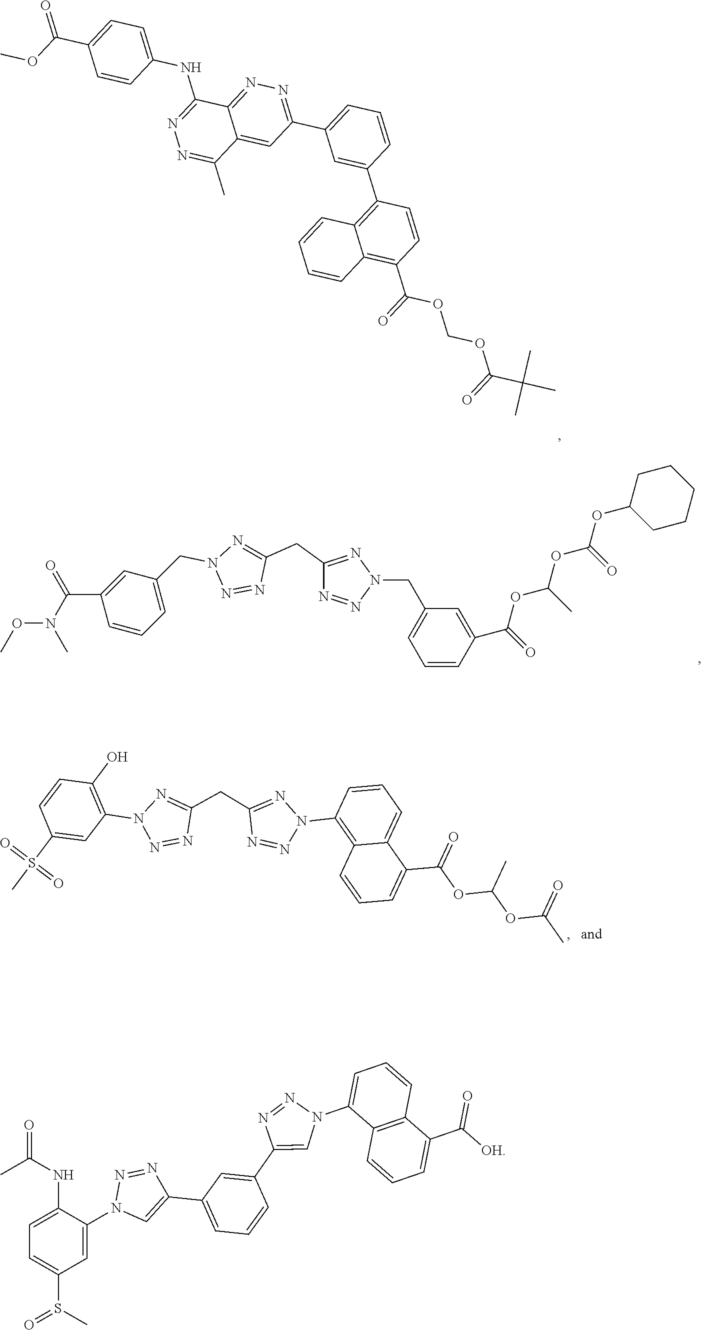

99. A compound selected from one or more of the following:
8-[4′-({5-[(2H-1,2,3,4-tetrazol-5-yl)methyl]-2H-1,2,3,4-tetrazol-2-yl}methyl)-[1,1′-
biphenyl]-4-amido]naphthalene-1-carboxylic acid,
5-{4-[hydroxy(1-{2-hydroxy-5-[methoxy(methyl)carbamoyl]phenyl}-1H-1,2,3-
triazol-4-yl)methyl]-1H-1,2,3-triazol-1-yl}naphthalene-1-carboxylic acid,
5-[4-(5-{1-[2-hydroxy-5-(methoxycarbonyl)phenyl]-1H-1,2,3-triazol-4-yl}pyridin-
3-yl)-1H-1,2,3-triazol-1-yl]naphthalene-1-carboxylic acid,
3-{[5-({2-[(2-butanamido-5-nitrophenyl)methyl]-2H-1,2,3,4-tetrazol-5-yl}methyl)-
2H-1,2,3,4-tetrazol-2-yl]methyl}naphthalene-1-carboxylic acid,
5-{4-[2-(1-{2-hydroxy-5-[methoxy(methyl)carbamoyl]phenyl}-1H-1,2,3-triazol-4-
yl)phenyl]-1H-1,2,3-triazol-1-yl}naphthalene-1-carboxylic acid,
5-{4-[2-(1-{2-acetamido-5-[methoxy(methyl)carbamoyl]phenyl}-1H-1,2,3-triazol-
4-yl)propan-2-yl]-1H-1,2,3-triazol-1-yl}naphthalene-1-carboxylic acid,
5-[4-(4-{1-[2-acetamido-5-(methoxycarbonyl)phenyl]-1H-1,2,3-triazol-4-
yl}phenyl)-1H-1,2,3-triazol-1-yl]naphthalene-1-carboxylic acid,
8-[4′-({5-[(2H-1,2,3,4-tetrazol-5-yl)methyl]-2H-1,2,3,4-tetrazol-2-yl}methyl)-[1,1′-
biphenyl]-4-amido]naphthalene-1-carboxylic acid,
5-[4-(3-{1-[2-acetamido-5-(methoxycarbonyl)phenyl]-1H-1,2,3-triazol-4-
yl}phenyl)-1H-1,2,3-triazol-1-yl]naphthalene-1-carboxylic acid,
5-[(4-{4-[1-({3-[methoxy(methyl)carbamoyl]phenyl}methyl)-1H-1,2,3-triazol-4-
yl]phenyl}-1H-1,2,3-triazol-1-yl)methyl]naphthalene-1-carboxylic acid,
5-[4-(4′-{1-[2-hydroxy-5-(methoxycarbonyl)phenyl]-1H-1,2,3-triazol-4-yl}-[1,1′-
biphenyl]-4-yl)-1H-1,2,3-triazol-1-yl]naphthalene-1-carboxylic acid,
5-[4-(6′-{1-[2-hydroxy-5-(methoxycarbonyl)phenyl]-1H-1,2,3-triazol-4-yl}-[3,3′-
bipyridine]-6-yl)-1H-1,2,3-triazol-1-yl]naphthalene-1-carboxylic acid,
N-{4-[4-(3-{1-[4-(acetamidosulfonyl)phenyl]-1H-1,2,3-triazol-4-yl}phenyl)-1H-
1,2,3-triazol-1-yl]benzenesulfonyl}-N-pentylacetamide,
5-(4-(3-(1-(2-acetamido-5-(methylsulfinyl)phenyl)-1H-1,2,3-triazol-4-yl)phenyl)-
1H-1,2,3-triazol-1-yl)-1-naphthoic acid,
3-[(5-{[2-({3-[methoxy(methyl)carbamoyl]phenyl}methyl)-2H-1,2,3,4-tetrazol-5-
yl]methyl}-2H-1,2,3,4-tetrazol-2-yl)methyl]benzoic acid,
5-[(6-chloro-3-{[(5-methanesulfonyl-1H-1,3-benzodiazol-2-yl)methyl]amino}-
1,2,4-triazin-5-yl)carbamoyl]naphthalene-1-carboxylic acid, and
2-[5-amino-3-(4-{4-[methoxy(methyl)carbamoyl]benzamido}-4-methylpiperidin-1-
yl)-1,2,4-triazin-6-yl]pyridine-4-carboxylic acid.

100. A prodrug of claim 67, wherein the prodrug is selected from one of more of the following compounds:

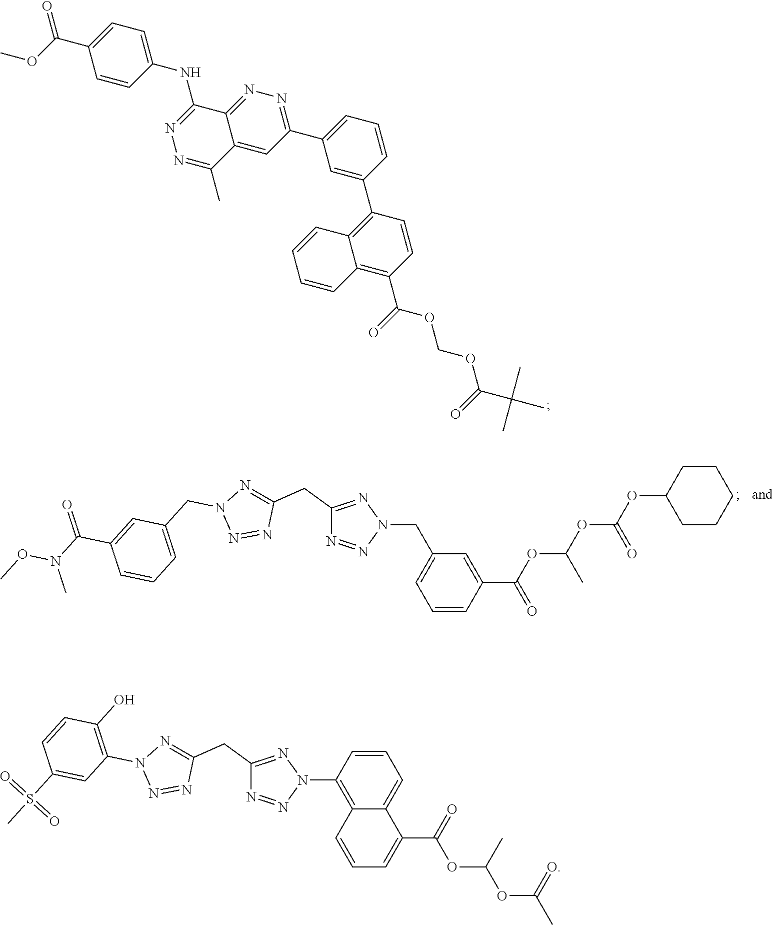

101. A pharmaceutical composition comprising the compound of any one of claims 67-100.

102. A method of treating, ameliorating, or preventing an autoimmune disease, or other immune-related diseases, atherosclerotic disorders, neurodegenerative disorders, inflammatory disease, prevention of transplantation rejection, and treatment of malignancies, comprising administering to a subject in need one or more compounds of claims 67-101.

103. The method of claim 102, wherein the disease is selected from one or more of systemic lupus erythematosus (SLE), rheumatoid arthritis, ankylosing spondylitis, lupus nephritis, Goodpasture's disease, Sjögren's syndrome, polymyositis, dermatomyositis, psoriasis, temporal arteritis, Churg-Strauss syndrome, multiple sclerosis, Guillain-Barré syndrome, transverse myelitis, myasthenia gravis, Addison's disease, thyroiditis, coeliac disease, ulcerative colitis, sarcoidosis, hemolytic anemia, idiopathic thrombocytopenic purpura, Behçet's disease, Alzheimer's disease, traumatic brain injury, chronic traumatic encephalitis, Parkinson's disease, angina, pectoris, myocardial infarction, primary biliary cirrhosis Crohn's disease, type 1 diabetes, Juvenile diabetes, autoimmune diabetes, acute and chronic rejection in bone marrow transplantation, graft versus host disease, acute and chronic rejection in orthotopic and heterotopic solid organ transplants, and prevention of rejection of bone marrow transplants.

104. A method of modulating tumor necrosis factor (TNF) super family comprising administering to a patient in need of treatment one or more compounds of claims 67-101.

105. The method of claim 104, wherein the compound or pharmaceutical composition, modulates one or more interactions selected from CD-40-CD40L, CD152,CD27-CD70, CD137(4-1BB)-4-1BBL, HVEM-LIGHT(CD258), CD30-CD30L, GITR-GITRL, BAFF-R(CD268)-BAFF(CD257), RANK(CD265)-RANKL(CD254), OX40(CD 134)-OX40L(CD252), and combinations thereof.

106. A method of treating, ameliorating, or preventing an autoimmune disease, or other immune-related diseases, atherosclerotic disorders, neurodegenerative disorders, inflammatory disease, prevention of transplantation rejection, and treatment of malignancies, comprising administering to a subject in need the compound of Formula III

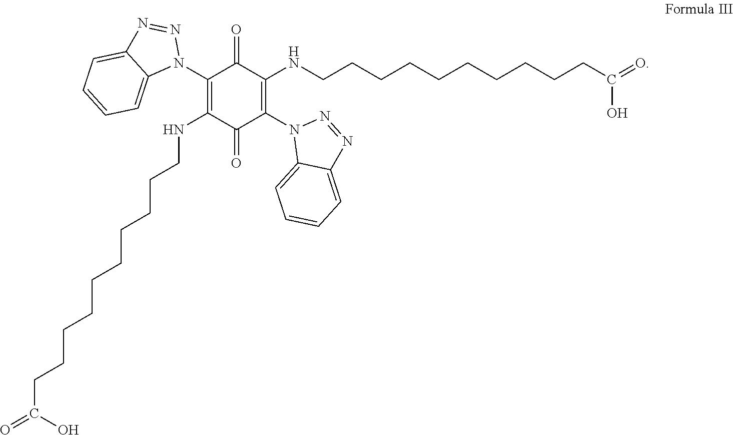 [SMILES: O=C(O)CCCCCCCCCCNC1=C(n2nnc3ccccc32)C(=O)C(NCCCCCCCCCCC(=O)O)=C(n2nnc3ccccc32)C1=O]

107. The method of claim 106, wherein the disease is selected from one or more of systemic lupus erythematosus (SLE), rheumatoid arthritis, ankylosing spondylitis, lupus nephritis, Goodpasture's disease, Sjögren's syndrome, polymyositis, dermatomyositis, psoriasis, temporal arteritis, Churg-Strauss syndrome, multiple sclerosis, Guillain-Barré syndrome, transverse myelitis, myasthenia gravis, Addison's disease, thyroiditis, coeliac disease, ulcerative colitis, sarcoidosis, hemolytic anemia, idiopathic thrombocytopenic purpura, Behçet's disease, Alzheimer's disease, traumatic brain injury, chronic traumatic encephalitis, Parkinson's disease, angina, pectoris, myocardial infarction, primary biliary cirrhosis Crohn's disease, type 1 diabetes, Juvenile diabetes, autoimmune diabetes, acute and chronic rejection in bone marrow transplantation, graft versus host disease, acute and chronic rejection in orthotopic and heterotopic solid organ transplants, and prevention of rejection of bone marrow transplants.

108. A method of modulating tumor necrosis factor (TNF) super family comprising administering to a patient in need of treatment the compound of Formula III

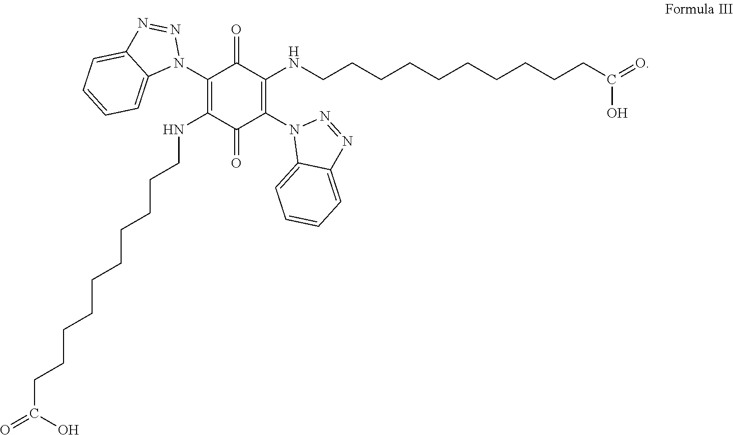 [SMILES: O=C(O)CCCCCCCCCCNC1=C(n2nnc3ccccc32)C(=O)C(NCCCCCCCCCCC(=O)O)=C(n2nnc3ccccc32)C1=O]

109. The method of claim 108, wherein the compound modulates one or more interactions selected from CD-40-CD40L, CD152,CD27-CD70, CD137(4-1BB)-4-1BBL, HVEM-LIGHT(CD258), CD30-CD30L, GITR-GITRL, BAFF-R(CD268)-BAFF(CD257), RANK(CD265)-RANKL(CD254), OX40(CD 134)-OX40L(CD252), and combinations thereof.
